(19) United States
(12) Patent Application Publication
(10) Pub. No.: US 2023/0165966 A1
(43) Pub. Date: Jun. 1, 2023

(54) METHODS FOR TREATING CANCER

(71) Applicants: BicycleRD Limited, Cambridge (GB); Cancer Research Technology, London (GB)

(72) Inventors: Maria KOEHLER, Cambridge (GB); Gillian Langford, Cambridge (GB); Stefan Symeonides, London (GB)

(21) Appl. No.: 17/861,838

(22) Filed: Jul. 11, 2022

Related U.S. Application Data
(62) Division of application No. 16/668,481, filed on Oct. 30, 2019, now abandoned.
(60) Provisional application No. 62/907,106, filed on Sep. 27, 2019.
(60) Provisional application No. 62/788,391, filed on Jan. 4, 2019.
(60) Provisional application No. 62/753,005, filed on Oct. 30, 2018.

(51) Int. Cl. A61K 47/64; A61K 47/68; A61P 35/00; C07K 16/28; C07K 16/30; G01N 33/574

(52) U.S. Cl. CPC A61K 47/6415; A61K 47/6857; A61P 35/00; C07K 16/2896; C07K 16/3023; G01N 33/57488

(57) ABSTRACT

The present invention relates to a method of treating cancer in a subject.

### CROSS-REFERENCE TO RELATED APPLICATIONS

The present application claims priority to U.S. provisional patent application Ser. No. 62/753,005, filed Oct. 30, 2018, U.S. provisional patent application Ser. No. 62/788,391, filed Jan. 4, 2019, and U.S. provisional patent application Ser. No. 62/907,106, filed Sep. 27, 2019, the entirety of each of which is incorporated herein by reference.

### BACKGROUND OF THE INVENTION

The membrane type I matrix metalloproteinase (MT1-MMP) protein is a member of the matrix metalloproteinase (MMP) family which are involved in tissue remodeling, mediated through proteolysis of collagen and other extracellular matrix components [1]. Overexpression of MT1-MMP in many solid tumors (including the surrounding stroma), is linked to cell invasion and migration [2]. This in turn is associated with poor prognosis and shorter survival in NSCLC [3, 4], breast cancer [5, 6] and other solid malignancies [7, 8, 9].

Matrix metalloproteinase inhibitors have been investigated but failed for various reasons such as poor pharmacology, metabolic stability, sub-optimal bioavailability and/or DLTs [10]. Using an alternative approach, BT1718 has been developed to take advantage of the overexpression of MT1-MMP, not to inhibit its activity, but as a cell surface target to selectively bind and facilitate delivery of the cytotoxic DM1 payload to the tumor.

For this study, the target population will be adult patients with advanced solid tumor malignancies refractory to all appropriate standard of care (SOC) treatment options. With the potential for benefit not exclusively restricted to a definitive subset of tumor types, or a definitive MT1-MMP expression level, dose escalation is planned to be open to patients of all solid tumor types. However, in the Phase IIa expansions at the optimal dose/schedule(s), a clinical signal will be explored in an enriched population with tumor types known to commonly over-express MT1-MMP and where MT1-MMP over-expression is confirmed during prospective selection at enrolment. These tumor types will be identified based on further pre-clinical data but are currently proposed to include tumors such as NSCLC, TNBC, ovarian, sarcomas, and tumor types expressing MT1.

### Non-Small Lung Cancer

Lung cancer is the second most common cancer in the United Kingdom (UK) and United States of America (USA) and the most common cause of cancer death in both countries. Outcomes are poor with just 10% having a 5-year survival. Non-Small Cell Lung Cancer represents 87% of all lung cancer in the UK. Only a small proportion of patients have early disease amenable to curative surgery and while more may be suitable for radical (chemo) radiotherapy, cure rates are low [11].

With advanced or relapsed disease, treatment is palliative and prognosis poor. A minority (<10%) of patients have tumors that can respond to mutation-directed treatments with epidermal growth factor receptor (EGFR) or anaplastic lymphoma kinase (ALK) inhibitors. Chemotherapy has been the mainstay of therapy for most patients, now being joined by immunotherapy. For patients who are suitable for first line platinum doublet chemotherapy, median overall survival (OS) is still just 11 months [12, 13]. Second-line chemotherapy, such as docetaxel, has modest activity for those fit enough to receive it, with an objective response rate (ORR) of 8 to 12% (docetaxel, pemetrexed) [14-18]. Antibodies and inhibitors targeting the programmed cell death protein 1 (PD-1) checkpoint blockade is now replacing chemotherapy in the second- (and now first-) line, with an ORR of around 20% (nivolumab, pembrolizumab, atezolizumab) for second-line treatment and at least 30% where there is programmed death—ligand 1 (PD-L1) selection [14-16, 18]. There is no SOC beyond these agents.

In this context, a promising new agent for NSCLC might therefore be expected to demonstrate an ORR greater than that seen with second-line chemotherapy comparators, aiming for 30% in a selected population. Membrane type I matrix metalloproteinase is highly expressed in NSCLC and BT1718 has shown excellent activity in multiple in vivo NSCLC models.

Triple Negative Breast Cancer

Triple Negative Breast Cancer is characterized by tumors that do not overexpress the estrogen receptor, the progesterone receptor, or the human epidermal receptor 2 (HER2). Triple Negative Breast Cancer cannot respond to current targeted agents, and also tends to behave more aggressively than other breast cancers and so represents an important area of unmet need. Around 10 to 15% of breast cancers diagnosed in the UK are classified as TNBC, accounting for >8000 cases per year [19].

With advanced or relapsed disease, treatment consists of palliative chemotherapy, with taxanes or platinums the most commonly used first line agents. These have an ORR of 30 to 35% but a progression free survival (PFS) of only around four months and OS of around 12 months [20-22]. There is no clear SOC for subsequent therapy but licensed third-line agents (e.g. capecitabine or eribulin) have an ORR of just 9-12% [23]. Fourth-line response rates may be as low as 2% [24]. New and more active agents are in clear need. Programmed cell death protein 1 checkpoint inhibitors have shown enough promise to proceed to Phase III trials, but with reported ORRs of <20%. An Antibody-Drug Conjugate (ADC) against glycoprotein NMB, CDX-011, has also shown some promise with an ORR of 30% [25] for NMB selected tumors in a small phase I expansion. Another ADC, sacituzumab govitecan (IMMU 132), has shown similar activity targeting Trop-2 [26]. Of interest, although in HER2 positive breast cancer, the ADC ado-trastuzumab emtansine (T-DM1) was first licensed in the third-line and had an ORR of 31% in this HER2 positive population [27].

As with NSCLC, a promising new agent for TNBC might therefore be expected to demonstrate an ORR greater than that seen with chemotherapy comparators, aiming for 30% in a selected population. Similar to NSCLC, MT1-MMP is highly expressed in TNBC and BT1718 has shown excellent activity in multiple in vivo TNBC models.

Sarcoma

Sarcomas develop in supporting or connective tissue and represent a broad range of different biological sub-types with an overall incidence of around six per 100,000 per year. Although not a common cancer (1% of all cancers), sarcoma represents an area of unmet need due to the lack of utility/refractory nature of agents in advanced disease [28, 29].

With advanced or relapsed disease, treatment consists of palliative chemotherapy (except in gastrointestinal stromal tumor (GIST), where KIT/platelet-derived growth factor (PDGF) receptor inhibitors are effective agents), with doxorubicin or ifosfamide the most commonly used first line agents. These have an ORR of just 10% to 25%. Taxanes, trabectedin and pazopanib are other agents available for specific sub-types of sarcoma but all with similarly limited benefit. There is no clear SOC beyond first-line therapy.

The European Organization for Research and Treatment Cancer (EORTC) Soft Tissue and Bone Sarcoma Group has established criteria [30] for determining promising new sarcoma therapeutics in early phase trials. Stable disease (SD) is included in this measure by defining a target PFS rate at three months. This is expected to be >20%, aiming for 40%. MT1-MMP is more highly expressed in sarcomas than any other cancer type from available data and BT1718 has shown excellent activity in an in vivo fibrosarcoma model.

### BRIEF DESCRIPTION OF THE FIGURES

FIG. 1A depicts the efficacy of BT1718 in the LU-010-0046 (high MT1-MMP expression) non-small-cell lung carcinoma (NSCLC) patient-derived xenograft (PDX) animal model dosed at 3 mg/kg BT1718 twice weekly and 10 mg/kg BT1718 twice weekly.

FIG. 1B depicts the efficacy of BT1718 in the LU-010-0251 (high MT1-MMP expression) non-small-cell lung carcinoma (NSCLC) patient-derived xenograft (PDX) animal model dosed at 3 mg/kg BT1718 twice weekly and 10 mg/kg BT1718 twice weekly.

FIG. 1C depicts the efficacy of BT1718 in the LU-010-0486 (low MT1-MMP expression) non-small-cell lung carcinoma (NSCLC) patient-derived xenograft (PDX) animal model dosed at 3 mg/kg BT1718 twice weekly and 10 mg/kg BT1718 twice weekly.

FIG. 2 depicts plasma drug concentration-time curves for BT1718 dosed at 0.015, 0.031, 0.061, 0.123, and 0.245 mg/kg/h. This dosing range can also be expressed as 0.6, 1.2, 2.4, 4.8 and 9.6 mg/m².

FIG. 3 depicts the study design schema.

FIG. 4 depicts a BT1718 clinical trial overview.

FIG. 5 depicts an overview of phase 1 and phase 2a.

FIG. 6 depicts patient status during BT1718 treatment.

FIG. 7A depicts increased tumor cell death following BT1718 dosing as shown by M30 and M65 assay for patient number 36/011.

FIG. 7B depicts increased tumor cell death following BT1718 dosing as shown by M30 and M65 assay for patient number 31/012.

FIG. 7C depicts increased tumor cell death following BT1718 dosing as shown by M30 and M65 assay for patient number 16/013.

FIG. 7D depicts increased tumor cell death following BT1718 dosing as shown by M30 and M65 assay for patient number 36/010.

FIG. 7E depicts increased tumor cell death following BT1718 dosing as shown by M30 and M65 assay for patient number 31/014.

FIG. 7F depicts increased tumor cell death following BT1718 dosing as shown by M30 and M65 assay for patient number 16/015.

FIG. 8 depicts the Dose Escalation Scheme including dose levels and patient numbers.

FIG. 9A depicts a spaghetti plot of BT1718 plasma concentration vs time after first doses in cycles 1 & 2 dosed at 0.6 mg/m², 1.2 mg/m², and 2.4 mg/m².

FIG. 9B depicts a spaghetti plot of BT1718 plasma concentration vs time after first doses in cycles 1 & 2 dosed at 4.8 mg/m², 7.2 mg/m², and 9.6 mg/m².

FIG. 9C depicts a spaghetti plot of BT1718 plasma concentration vs time after first doses in cycles 1 & 2 dosed at 15 mg/m², 20 mg/m², and 25 mg/m².

FIG. 10A depicts a scatter plot of BT1718 AUC vs dose.

FIG. 10B depicts a scatter plot of BT1718 Cmax vs dose.

### DETAILED DESCRIPTION OF CERTAIN EMBODIMENTS OF THE INVENTION

### 1. Compound

A proprietary phage display and cyclic peptide technology (Bicycle® technology) was utilized to identify high affinity binding peptides to the membrane type 1-matrix metalloproteinase (MT1-MMP/MMP14). MT1-MMP (MT1) is a cell surface membrane protease normally involved in tissue remodeling which has been found to be over-expressed in many solid tumors. Overexpression of MT1 has been linked to cancer invasiveness and poor prognosis. While attempts to target the proteolytic activity of MT1 and other MMPs in cancer were unsuccessful in clinical trials largely due to toxicity caused by insufficient selectivity, MT1-MMP remains an attractive cancer target for targeted cytotoxic delivery approaches.

Diverse selection phage libraries containing 10¹¹ to 10¹³ unique peptide sequences which are post-translationally cyclized with thiol-reactive scaffolds were used to identify small (1.5-2 kDa) constrained bicyclic peptides binders (Bicycles) to the hemopexin domain of MT1. Initial binders were subject to affinity maturation by directed screens and stabilization by chemical optimization.

A bicyclic constrained peptide binder (Bicycle) was identified that binds to the hemopexin domain of MT1 with an apparent Kd of approximately 2 nM. The Bicycle peptide (N241) binds with similar affinity to the entire ectodomain of the protease but shows no binding to the catalytic domain. N241 also shows no binding toward any of the closely related MMP family members tested (MMP15, MMP16, MMP24, MMP1, Pro-MMP1, MMP2). Characterization of the pharmacologic effect of N241 on MT1 in vitro shows that the peptide has no direct impact on the catalytic activity of the protease, nor related MMP catalytic activity (MMP1, MMP2 and MMP9) nor cell migration or invasion. However, binding of fluorescently-tagged N241 to MT1 on HT1080 fibrosarcoma cells results in the rapid internalization and subsequent lysosomal localization of the compound. In addition, ¹⁷⁷Lu-loaded N241 demonstrates rapid tumor localization when injected IV into mice bearing MT1-positive tumor xenografts, with levels as high as 15-20% injected dose per gram of tumor in less than 60 minutes. In contrast, a non-binding Bicycle peptide shows no tumor localization. These properties suggest that N241 may be a good delivery vehicle for cytotoxic payloads targeting MT1-positive tumor cells. Bicycle drug conjugates (BDCs) with a variety of linkers and cytotoxic payloads were prepared which retained binding to MT1. The anti-tumor activity of select BDCs was demonstrated in MT1-positive human tumor cell xenografts in mice.

BT1718 is a Bicycle drug conjugate (BDC) comprising a constrained bicyclic peptide that binds with high affinity and specificity to membrane type 1-matrix metalloprotease (MT1-MMP; MMP14) covalently linked through a hindered disulfide linker to the potent anti-tubulin agent DM1. MT1-MMP is naturally involved in tissue remodeling, however overexpression of the cell-surface protease has been tied to tumor aggressiveness and invasiveness, as well as poor patient prognosis for many cancer indications. The Bicycle binder for BT1718 (N241) was identified using a proprietary phage display peptide technology consisting of highly diverse phage libraries of linear amino acid sequences constrained into two loops by a central chemical scaffold. While binding with similar affinity and specificity to that observed with monoclonal antibodies, the small size of a Bicycle peptide (1.5-2 kDa) aids in its rapid extravasation and tumor penetration making it an ideal format for the targeted delivery of cytotoxic payloads.

A series of maytansinoid-BDC conjugates were prepared, with varying linker format to adjust cleavability and evaluated for their anti-tumor activity in an MT1-positive tumor xenograft model. The BDC selected for further assessment (BT1718) was evaluated for efficacy in an array of tumor xenograft models.

A mono-hindered linker-DM1 construct (BT1718) was among the most active constructs against MT1-positive EBC-1 lung tumor xenografts. Efficacy in this model was reduced in the conjugates containing the least cleavable linkers. Dosing BT1718 on a twice weekly schedule for two weeks, significant reduction in tumor growth was seen at 3 mg/kg, with 10 mg/kg causing complete regressions in this model. Effective treatment was also seen with same total dose, given at on schedules from daily to a single weekly dose. Treatment with BT1718 in a selection of MT1-positive tumor xenograft models (e.g. HT1080 fibrosarcoma; HCC1806 triple negative breast cancer; SNU-16 gastric cancer) demonstrated activity at minimally effective doses in the range of 3-10 mg/kg weekly or twice weekly, with 10 mg/kg twice weekly causing complete regressions in most models. Preliminary metabolism studies indicate that BT1718 is excreted mainly through the kidney in urine.

BT1718, a Bicycle drug conjugate (BDC), shows potent antitumor activity in human tumor xenograft models of fibrosarcoma, lung and breast cancer. Without wishing to be bound by any particular theory, it is believed that the small size of the BDC may offer a significant advantage to other targeted cytotoxic approaches such as antibody-drug conjugates due to rapid extravasation and improved tumor penetration.

BT1718 is a potent, highly selective Bicycle Drug Conjugate (BDC) consisting of a novel bicyclic peptide (Bicycle), which binds selectively to membrane type 1-matrix metalloproteinase (MT1-MMP), which is connected through a molecular spacer and a cleavable disulfide linker to the potent cytotoxic tubulin inhibitor, DM1. Upon binding to tumor cells expressing MT1-MMP, the DM1 payload is activated by release from the conjugate where it can disrupt microtubule dynamics resulting in tumor cell death.

BT1718 is a potent, highly selective BDC developed by BicycleRD using their novel platform technology of constrained bicyclic peptide binders, from herein referred to as Bicycles. The BDCs, have a low molecular weight (3.5 kDA) in comparison to other conjugated toxin approaches, which enables rapid penetration of tumor tissue. Minimal systemic toxicity is expected due to its short half-life and excretion via the kidneys, potentially sparing gastrointestinal (GI) and hepatic toxicity, a frequent on target toxicity seen with small molecule cytotoxics and ADCs. BT1718 specifically binds to cell-surface MT1-MMP overexpressed on tumor cells, which facilitates delivery of its cytotoxic payload, DM1, to the tumor. Once released by tumor-localized cleavage of the linker, active unconjugated DM1 is then able to block normal microtubule function during cell division, ultimately leading to apoptosis, cell death and reduction of tumor size.

In certain aspects, the present invention provides a method of treating certain cancers in a subject, comprising administering to the subject an effective amount of a drug conjugate comprising a high affinity binder of MT1-MMP, such as BT1718, or a pharmaceutically acceptable salt or composition thereof.

Preparation of BT1718 is described in detail in WO 2016/067035, filed Oct. 29, 2015, the entirety of which is hereby incorporated herein by reference. BT1718 has the structure shown below:

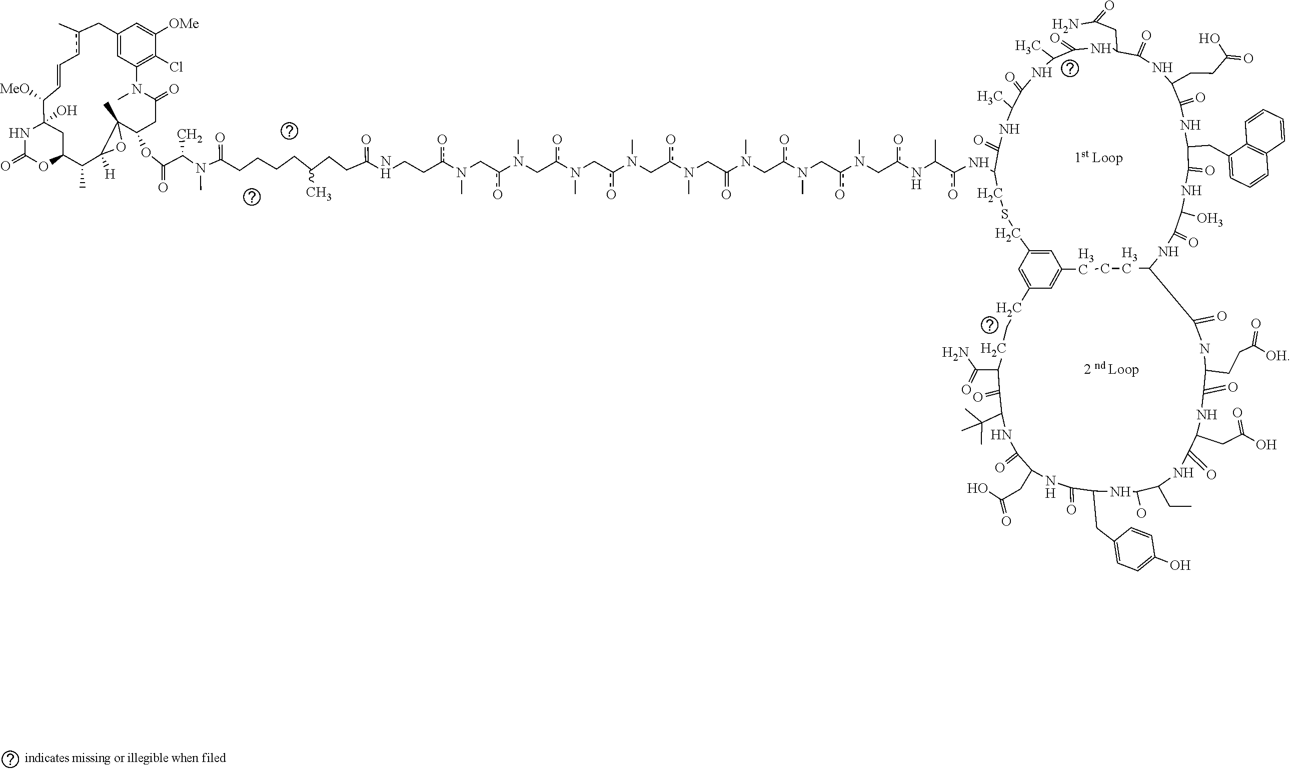

### 2. Definitions

As used herein, the terms “about” or “approximately”, used in conjunction with a numerical value, refer to a range by extending the boundaries above and below the numerical values. For example, the terms “about” or “approximately” can extend the stated value by a variance of 30%, 25%, 20%, 15%, 10%, 9%, 8%, 7%, 6%, 5%, 4%, 3%, 2%, 1%, or 0.5% up and/or down (higher or lower). In some embodiments, the terms “about” or “approximately” extend the stated value by a variance of 25% up and/or down (higher or lower). In some embodiments, the terms “about” or “approximately” extend the stated value by a variance of 10% up and/or down (higher or lower). In some embodiments, the terms “about” or “approximately” extend the stated value by a variance of 5% up and/or down (higher or lower).

### Safety Considerations

Non-Clinical Pharmacology

BT1718 is a BDC that binds to MT1-MMP and upon cleavage of its disulphide linker, releases the cytotoxic tubulin inhibitor, DM1 (also referred to as DM1-SH). BT1718 has high affinity and selectivity for the hemopexin domain of MT1-MMP (inhibitory constant, Ki 1.75±0.92 nM), with the Bicycle component having no significant binding for the catalytic domain of MT1-MMP (at least 50-fold selectivity), nor other related-MMP hemopexin domains (over 200-fold selectivity). In addition, BT1718 does not interfere with the proteolytic activity of MT1-MMP.

Membrane type I matrix metalloproteinase binding Bicycles have been shown to bind to cell surface MT1 MMP and are internalized with subsequent localization to the lysosomal compartment. BT1718 demonstrated potent cell killing activity (nM IC₅₀s range) towards MT1-MMP-expressing tumor cells which was at least 2-orders of magnitude greater than for cells which did not express MT1-MMP; whereas all cells, irrespective of their MT1-MMP expression levels, showed similar sensitivity to unconjugated DM1 alone. In addition, excess Bicycle component of BT1718 antagonized BT1718 cytotoxicity. Taken together, these data demonstrate that BT1718 binds to MT1-MMP-expressing cells and mediates target-dependent cell killing.

In vivo, intravenous administration of BT1718 showed dose-dependent anti-tumor activity with disease stabilization and/or regression in multiple xenograft models representing a variety of tumor types including lung, breast, gastric, fibrosarcoma nasal and colorectal. The minimum effective dose in the EBC-1 model, was 1 mg/kg (3 mg/m²) three times a week (slowed tumor growth) and in the HT-1080 model was 2 mg/kg (6 mg/m²) given twice weekly (Table 1). A dose of 3 mg/kg (9 mg/m²) twice weekly produced SD or better in several models and the highest dose tested, 10 mg/kg (30 mg/m²) twice weekly caused complete regressions in the majority of MT1-MMP-expressing xenograft tumors tested (Table 1), often with mice remaining tumor-free to the end of the study. In some instances, where re growth of tumors occurred after a suspension of dosing, administration of BT1718 was still able to induce significant regressions or reduced tumor growth upon re administration. BT1718 anti-tumor activity in the xenograft models tended to correlate with cytotoxic potency observed in the in vitro assays. Furthermore, and as demonstrated in vitro, excess unconjugated MT1-MMP binding Bicycle attenuated the anti-tumor effect suggesting that MT1 MMP binding is important for BT1718 efficacy in vivo. Consistent with this finding, immunohistochemistry (IHC) of xenograft tumors confirmed that the models in which BT1718 produced the greatest anti-tumor effect showed strong membrane staining for MT1-MMP, while models that did not respond as well to BT1718 (SD at the highest dose) had limited or no membrane MT1-MMP expression.

The relationship between anti-tumor activity and dosing schedule for BT1718 was investigated. Anti-tumor activity was compared when equivalent total weekly doses were administered either once, twice or three times weekly or once daily. At a high total weekly dose of 60 mg/m², all schedules resulted in complete regression of the tumor and were well tolerated, except for 60 mg/m² once weekly which caused notable weight loss (>10%). At a lower dose level (total weekly dose of 18 mg/m²), the once weekly and twice weekly schedules were the most effective, with the more frequent schedules (three times weekly or once daily) showing somewhat reduced anti-tumor effects. In summary, the non-clinical data supports the clinical evaluation of different schedules such as once weekly. Additionally, less frequent dosing schedules may be evaluated.

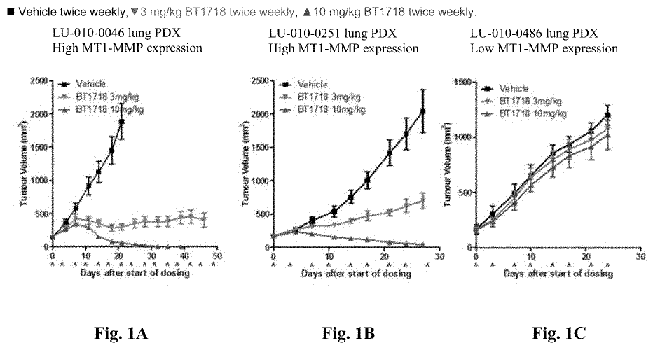

Data generated in NSCLC patient-derived xenograft (PDX) models that did not express high levels of MT1-MMP showed no anti-tumor activity in response to BT1718. However, MT1-MMP-expressing PDX models were sensitive to BT1718; with 3 mg/kg (9 mg/m²) twice weekly, producing SD, whereas; vehicle control animals all demonstrated progressive disease (PD) with tumor growth in excess of 2,000 mm³ by Day 27 (FIG. 1A, FIG. 1B and FIG. 1C). Furthermore, BT1718 produced an anti-tumor effect against MT1 MMP-expressing NSCLC PDX models irrespective of their sensitivity to the SOC chemotherapeutic docetaxel. BT1718 (10 mg/kg; 30 mg/m² twice weekly) caused complete regression of tumors in both models, whereas clinically relevant doses of docetaxel (60 mg/m² once weekly) produced regression only in the docetaxel-sensitive model. A lower dose of BT1718 (3 mg/kg; 9 mg/m² twice weekly) produced SD in both models. The PDX models confirm that MT1-MMP expression is necessary for BT1718 anti-tumor activity.

Summary of In Vitro and In Vivo Anti-Tumor Activity

TABLE 1
Summary of in vitro anti-tumor activity

| | | MT1-MMP | |
|---|---|---|---|
| | | Expression | Cytotoxicity |
| Cell line | Type | H-score | IC₅₀ nM |
| EBC-1 | Lung | 38 ± 9 | 27 |
| HT-1080 | Fibrosarcoma | 67 ± 9 | 4.3 |
| NCI-H292 | NSCLC | NM | 11.6 |
| NCI-H1975 | NSCLC | NM | 19.7 |
| MDA-MB- | Breast | 70 ± 3 | 39.8 |
| 231 | | | |
| HCC-1806 | Breast | NM | 1.4 |
| RPMI2650 | Nasal | NM | NM |
| HCT-15 | Colorectal | NM | 869.9 |
| HT-29 | Colorectal | 0 | 13.0 |
| SNU-16 | Gastric | NM | 19.5 |
| MOLP-8 | Myeloma | 3 ± 0 | 141.1 |

TABLE 2
Summary of in vivo anti-tumor activity.

| | in vivo anti-tumor activity | | | | |
|---|---|---|---|---|---|
| | tumor | TGI % on Day 14 | | | |
| | size | 1 mg/kg | 3 mg/kg | 10 mg/kg | |
| Cell line | mm³ | (3 mg/m²) | (9 mg/m²) | (30 mg/m²) | Description of anti-tumor effect |
| EBC-1 | 150 | 38 | 91*** | 114*** | Complete regressions at 10 mg/kg |
| | | | | | by Day 14; significant TGI at |
| | | | | | 3 mg/kg |
| HT-1080 | 180 | 27 | 109*** | 108*** | Complete regressions at ≥ 3 mg/kg |
| | | | | | by Day 14; no re-growth at |
| | | | | | 10 mg/kg |
| NCI-H292 | 150 | 21 | 41 | 102*** | Near complete regressions at |
| | | | | | 10 mg/kg by Day 30 |
| NCI-H1975 | 170 | 3 | 11 | 81 *** | Near complete regressions at |
| | | | | | 10 mg/kg by Day 25 |
| MDA-MB- | 187 | 8 | 26** | 102*** | Near complete regressions at |
| 231 | | | | | 10 mg/kg by Day 25 |
| HCC-1806 | 189 | 9 | 69 ** | 111*** | Complete regressions at 10 mg/kg |
| | | | | | by Day 18; significant TGI at |
| | | | | | 3 mg/kg |
| RPMI2650 | 147 | 4 | 20 | 102*** | Stable disease at 10 mg/kg by |
| | | | | | Day 14 |
| HCT-15 | 157 | −0.8 | 5 | 33 | Tumor progression at all doses |
| HT-29 | 160 | 24 | 7 | 103* | Stable disease at 10 mg/kg until |
| | | | | | Day 22 |
| SNU-16 | 160 | 51 | 95** | 155*** | Complete regression at 10 mg/kg by |
| | | | | | Day 14; near stable disease at |
| | | | | | 3 mg/kg |
| MOLP-8 | 169 | 37.5 | 59.2 * | 99.4 ** | Stable disease by Day 14 no |
| | | | | | regression |

Table 1 and Table 2 Notes: in general regression and stable disease correlated with the in vitro cytotoxicity and MT1-MMP expression. Female BALB/c mice n=3/group animals dosed twice per week except EBC-1 three per week. Tumor growth inhibition (TGI)(%)=[1−(Ti−T0)/(Vi−V0)]×100; Ti is the average tumor volume of a treatment group on a given day, T0 is the average tumor volume of the treatment group on the day of treatment start, Vi is the average tumor volume of vehicle control group on the same day with Ti, and V0 is the average tumor volume of vehicle group on the day of treatment start. A one-way ANOVA was performed to compare tumor volume among groups, and when a significant F-statistics (a ratio of treatment variance to the error variance) was obtained, comparisons between groups were carried out with Games-Howell test. All data were analyzed using GraphPad Prism. *p<0.05, **p<0.01, ***p<0.001. In vitro cytotoxicity was measured by CellTiter-Glo®, MT1-MMP membrane H score from immunohistochemistry staining, NM not measured. Tumor size on Day 1 of dosing.

MT1-MMP Expression Dependent Anti-Tumor Activity in Non-Small Cell Lung Cancer Patient-Derived Xenograft Models

Patient derived xenograft (PDX) lung models were chosen for their MT1-MMP expression. Mice were dosed twice weekly with vehicle 3 mg/kg or 10 mg/kg twice weekly. BT1718. Mean±SEM, n=6/group. Tumor growth inhibition (TGI) was calculated on Day 21-27 dependent on model, a one-way ANOVA was performed to compare tumor volume among groups, and when a significant F-statistics (a ratio of treatment variance to the error variance) was obtained, comparisons between groups were carried out with Games-Howell test. TGI 91%, 71% and 11% for 3 mg/kg dosed animals in LU 01 0046, LU-01-0251 and LU-01-0486 respectively and in 10 mg/kg groups TGI was 105%, 106% and 17% respectively. MT1-MMP expression dependent anti-tumor activity in Non-Small Cell Lung Cancer Patient-Derived Xenograft models is depicted in FIG. 1A, FIG. 1B, and FIG. 1C.

As part of the Good Laboratory Practice (GLP)-compliant toxicity studies, a core battery of safety pharmacology tests were performed. No effects were seen in the central nervous system (CNS) behavioral model (Irwin study) following a single dose of BT1718 to rats at a dose level of 0.2, 0.5 or 1 mg/kg (1.2, 3, 6 mg/m² respectively). Electrocardiogram (ECG) and respiratory plethysmography measurements were taken in monkeys dosed at 0.2, 0.5, 1 and 1.5 mg/kg (2.4, 6, 12 and 18 mg/m² respectively) twice weekly on Day 4, Day 22 (following 2nd and 7th dose) and in the recovery period (for animals dosed with 0, 6 and 12 mg/m² twice weekly). Blood pressure (BP) measurements were also evaluated in the same monkeys on Day 1 and Day 25 and in the recovery period. Safety studies conducted to GLP showed no BT1718 related findings in any of the parameters measured on any day. It is therefore concluded that BT1718 has no cardiac, respiratory or behavioral liability at the doses and schedule tested in the GLP toxicity studies.

In conclusion, BT1718 produces anti-tumor efficacy in xenografts and PDX models that express MT1 MMP with long term regression. Based on the available preclinical data, dosing twice weekly appeared to be the most tolerated and efficacious dosing schedule. In GLP-compliant safety pharmacology studies, no BT1718-related effects on cardiovascular, respiratory or behavioral function were observed up to the highest dose tested (18 mg/m²). BT1718 is a novel delivery platform that can deliver DM1 to MT1-MMP expressing cells, which are commonly found in cancers such NSCLC, TNBC and sarcoma.

Pharmacokinetics

The stability of BT1718 was assessed in plasma and whole blood from humans, monkey, dog, mouse and rat. The half-life of BT1718 at 37° C. in the plasma in vitro was >6 hours in human, rat and dog, >5 hours for mouse and monkey. The half-life of BT1718 at 37° C. in whole blood in vitro was >24 hours in mouse, rat and dog, 2 to 4 hours for human and monkey. The mechanism for the difference in stability of the BT1718 peptide in whole blood between the different species is unknown. BT1718 showed limited distribution into blood cells (in line with an expected low uptake into cells lacking MT1-MMP expression) which was comparable across human and all preclinical species tested.

Plasma protein binding of BT1718 was also assessed. The mean plasma protein binding of the BT1718 peptide ranged from 87% to 98%, with the free fraction of BT1718 being 13% in human, 2.6% in rats, 7.5% in mice, 3.8% in monkey and 1.5% in dog plasma, the results had no correlation to the plasma and whole blood stability data. BT1718 showed limited distribution into blood cells (in line with an expected low uptake into cells lacking MT1-MMP expression) which was comparable across human and all preclinical species tested. Plasma protein binding of unconjugated DM1, has previously been reported, with the unbound fraction being 7% in human and monkey and 3% in rat [31, 32]. Preliminary biodistribution studies and recovery in the PK studies suggest that BT1718 is primarily cleared by the kidney and therefore the plasma protein binding differences are not likely to be clinically highly significant from a safety perspective.

The PK properties of BT1718 in rodents were characterised by a moderate volume of distribution, a fast plasma clearance, and a short half-life (Table 3). The area under the curve (AUC) was dose proportional in CD1 mice.

In the lung cancer xenograft (EBC-1) study PK samples were also analysed and a dose of 10 mg/kg (30 mg/m²) was associated with a maximum exposure at the first time point measured (5 minutes) was 7.6±0.6 μM. In a study in CD1 mice a dose of 10 mg/kg (30 mg/m²) yielded an extrapolated C₀ of 9.3 μM and with an AUC 5.3 μM·h which will be a more reliable measure of exposure. A dose of 3 mg/kg (9 mg/m²) produced SD in several xenograft models in the EBC-1 model this was associated with a maximum observed plasma concentration (Cₘₐₓ) of 1.5±0.9 μM. In BALB/c mice a Cₘₐₓ of 8 μM and an AUC ˜2.5 μM·h were measured at a dose of 5 mg/kg (15 mg/m²). In the scheduling experiment in EBC-1 xenograft mice a dose of 20 mg/kg (60 mg/m²) was associated with significant weight loss; in CD1 mice this was associated with a Cₘₐₓ of 38.2 μM and an AUC of 11.6 μM·h. The dose-dependent efficacy is supported by the dose-dependent exposure to BT1718.

In a repeat dose experiment with BT1718 no significant gender-related differences in the TK parameters for the rat or monkey were noted. Exposure in both the rat and monkey increased approximately linearly with dose and was maintained for 28 days on repeat dosing. Plasma t_{1/2} was between 0.2 and 0.56 hours irrespective of dose. Plasma clearance and volume of distribution were similar across BT1718 dose and consistent with the PK studies.

TABLE 3
Summary Pharmacokinetic and Toxicokinetics parameters.

| | | | | | | Cl |
|---|---|---|---|---|---|---|
| | Dose | C₀/Cₘₐₓ | AUC | t_{1/2} | Vdₛₛ | (mL/min/ |
| | administered | μM | μM/h | hours | (L/Kg) | kg) |
| EBC-1 | 9 mg/m² | 1.5 ± 0.9 | NA | NC | NC | NC |
| xenograft | | | | | | |
| EBC-1 | 30 mg/m² | 7.6 ± 0.6 | NA | NC | NC | NC |
| xenograft | | | | | | |
| BALB/c mouse | 15 mg/m² | 7.2 ± 0.41 | 2.3 ± 0.2 | 0.56 ± 0.18 | 0.23 ± 0.09 | 7.5 ± 0.6 |
| nu/nu BALB/c | 15 mg/m² | 5.8 ± 0.94 | 1.7 ± 0.3 | 0.43 ± 0.04 | 0.26 ± 0.08 | 10.6 ± 1.5 |
| mouse | | | | | | |
| CD1 mouse | 15 mg/m² | 9.3 | 2.8 | 0.33 | 0.20 | 8.4 |
| CD1 mouse | 30 mg/m² | 9.3 | 5.3 | 0.34 | 0.27 | 9.0 |
| CD1 mouse | 60 mg/m² | 38.2 | 11.6 | 0.34 | 0.22 | 8.2 |
| Sprague Dawley | 15 mg/m² | 4.2 ± 1.1 | 1.5 ± 0.2 | 0.56 ± 0.11 | 0.29 ± 0.03 | 9.4 ± 1.1 |
| rat | | | | | | |
| Crl:WI(Han) | 1.2 mg/m² | 0.1 ± 0.01* | NC | NC | NC | NC |
| rats | | | | | | |
| Crl:WI(Han) | 6 mg/m² | 0.4 ± 0.14* | NC | NC | NC | NC |
| rats | | | | | | |
| Wister Han rats | 1.2 mg/m² | 0.08 | 0.09 | NC | NC | NC |
| Wister Han rats | 3 mg/m² | 0.59 | 0.46 | NC | NC | NC |
| Wister Han rats | 6 mg/m² | 1.02 | 0.85 | NC | NC | NC |
| Monkeys | 2.4 mg/m² | 0.20 ± 0.04 | 0.08 ± 0.02 | 0.21 | 0.24 ± 0.05 | 13.4 ± 4.7 |
| Monkeys | 6 mg/m² | 0.67 ± 0.04 | 0.28 ± 0.03 | 0.23 | 0.17 ± 0.02 | 8.8 ± 0.8 |
| Monkeys | 12 mg/m² | 1.21 ± 0.09 | 0.59 ± 0.14 | 0.36 | 0.21 ± 0.01 | 8.2 ± 0.6 |
| Monkeys | 18 mg/m² | 2.53 | 1.09 | 0.38 | 0.16 | 6.7 |

Table 3
Notes:
mean ± Standard deviation; All studies were intravenous; Cₘₐₓ, maximum concentration; C₀ extrapolated Cₘₐₓ at time 0; AUC, area under the curve; CL, clearance; t_{1/2} terminal half life; Vdₛₛ volume distribution at steady state, NC not calculated. EBC- 1 xenografts study females mice Cₘₐₓ is 5 minute timepoint , BALB/c PK study, females mice only, CD1 male mice dose response study and Sprague Dawley male rat study, Wister Han rats non GLP tox study, male Day 1 numbers presented only
*Cₘₐₓ is 1 hour time point, Wistar Han rats GLP study, values are Day 1 male values no significant difference between sex or on repeat dosing, due to the composite nature of the study design mean Cₘₐₓ, AUC and Tₘₐₓ were calculated only. Monkey GLP study values are Day 1 male values no significant difference between sex or on repeat dosing. Mean and median Cmax/dose was 1.5 across all pharmacokinetics studies in both mice and rats and the AUC/dose was 0.5.

The concentration of total DM1 in plasma and urine was also measured following a reduction step, to detect DM1 in BT1718, any peptidyl-DM1 metabolites of BT1718, other DM1-containing mixed disulfides and free DM1. The level of total DM1 in plasma and urine increased with increasing dose, with about 85% of the total DM1 recovered in urine within 48 hours of BT1718 administration. Little intact BT1718 was recovered in urine, suggesting that the Bicycle component of BT1718 may undergo proteolytic cleavage. The high recovery of total DM1 in urine suggests that most DM1 that is excreted is still conjugated to a fragment of the Bicycle (peptidyl-DM1), as free DM1 has been demonstrated to be cleared through the liver and excreted in bile and faeces and undergoes extensive metabolism by phase 1 and phase 2 enzymes [33]. An assay for free DM1 has not been successfully developed due to the instability of the free thiol in biological matrices.

Photoacoustic studies demonstrated rapid tumor penetration in comparison the MT1-MMP specific antibody. In addition, biodistribution and organ distribution studies of radiolabelled Bicycle in tumor-bearing mice showed rapid accumulation of radioactivity in the tumor, peaking at 12.02±2.37% initial dose/gram (% ID/g) 1 hour post-dose before decreasing to 1.54±0.06% ID/g at 24 hours, with rapid clearance from the vasculature (within 20 minutes). Accumulation of the majority of the radioactivity was found in the kidney and bladder irrespective of the labelled peptide dose; this is indicative of either non-saturable excretion or proteolysis followed by excretion through the kidneys. Furthermore, the intrinsic clearance of BT1718 and its Bicycle component, N241 was low when measured in cryopreserved hepatocytes. The negligible metabolism by the hepatocytes and the distribution of the Bicycle component as seen in in vivo imaging/radiolabelled studies, taken together with the urinary of total DM1 in the preliminary PK studies, would suggest a very different excretion and metabolism pathway to unconjugated DM1. Unconjugated DM1, when radiolabelled, (as [[³H]-DM1 (91 μCi/kg, 200 μg/kg, IV), was reported to distribute rapidly and extensively to the lungs, liver, kidney, spleen, heart, gastrointestinal tract, and adrenal glands in the rat. The major route of excretion of DM1 was through the bile/faeces, with a minimal amount excreted in the urine [33], a completely different distribution to BT1718 and its metabolites with little and short lived accumulation seen in other organs such as heart, liver, GI and lungs which would imply that these organs may be spared systemic toxicity.

BT1718 has a fast plasma clearance resulting in a short half-life. BT1718 is either excreted as the intact parent or as proteolytic fragments/metabolites by the kidney in preclinical studies. As such BT1718 has a very different excretion and metabolism pathway to free DM1. In addition, Bicycles have been shown to penetrate the tumor within 20 minutes of administration unlike high molecular weight ADCs.

Toxicology

A program of in vivo preclinical safety evaluation studies have been conducted with BT1718 to support the clinical use of intravenously administered BT1718. These include in vitro tissue microarray in human, rat and monkey tissues and in vitro and in vivo assessment of immunogenicity. In vivo assessments include single dose toxicity in mice and rats, multiple dose range finding studies in mice, rats, dogs and monkeys (non-GLP) and GLP-compliant multiple dose studies in rats and monkeys consisting of 28-day on study phase with a 28-day recovery period. The Bicycle component of BT1718, N241, elicited positive responses in only 2% of donor cohort (n=50) in the in vitro immunogenicity tests indicating a low risk of clinical immunogenicity, which was supported by all serum samples being negative for anti-drug antibodies (ADAs) in both the rat and monkey 28-day GLP compliant studies. The key target organs following administration of BT1718 have been identified as the haematopoietic and lymphoid tissue system, kidney and, bladder, liver, neuronal nervous system, skin (changes were generally in the vicinity of the injection site) and to a lesser extent other highly replicating tissues, such as reproductive organs, GI and secretory cells such as adrenal, pancreatic and salivary tissues. All toxicities were dose-dependent, reversible or showed signs of recovery during the recovery phase, with the exception of the minimal axonal degeneration and reproductive organ changes.

In a GLP-compliant study in Cynomolgus monkeys, the highest non-severely toxic dose (HNSTD) of BT1718 was established to be 18 mg/m² administered twice weekly, equivalent to a human dose of 0.48 mg/kg. In a GLP-compliant study in the rat, the MTD was not reached with the highest administered dose 6 mg/m² twice weekly. At this dose level, there was kidney toxicity (with no effect on clinical condition) and also testicular toxicity. After 28 days off-dose, there was evidence of partial recovery of kidney-related findings, but no recovery of testicular effects at this dose level. In the non-GLP rat study a dose of 9 mg/m² administered twice weekly was not well tolerated. Therefore, the MTD for rat for twice weekly dosing was considered to be between 1 and 1.5 mg/kg.

Target tissues for BT1718 toxicity across the GLP and non-GLP studies were as follows:

Hematological Changes

Changes in haematology were observed in rats and dogs and monkeys administered BT1718.

Reversible thymus atrophy and decreased germinal centres in lymph nodes were seen in monkeys administered 1 mg/kg or 1.5 mg/kg (12 mg/m² or 18 mg/m²) twice weekly. There was reversible reduction in white blood cell (WBC) and reticulocytes with an onset of 0.5 mg/kg (6 mg/m²) twice weekly and reticulocytes with an onset of 1 mg/kg (12 mg/m²) twice weekly during the dosing period (maximum 0.11× and 0.2× respectively) and the reduction in reticulocyte was accompanied by dose-dependent but minimal reduction of erythrocytes and parameters (0.63 to 0.93×). A dose-dependent increase in platelets (1.4-2.5×) was seen in monkeys administered 1 mg/kg (12 mg/m²) twice weekly or above.

In the rat GLP study, administration of BT1718 at 1 mg/kg (6 mg/m²) twice weekly caused a reduction in reticulocytes (0.06× in males and 0.37× in females) and erythrocyte parameters (0.64× maximum drop), as well as an increase in platelets (2.3× increase maximum). A loss of cellularity and lymphoid depletion in lymph nodes were present, however unlike in the monkey, no leukocyte decreases were observed. All hematological toxicities were reversible.

In non-GLP studies, changes in peripheral haematology were observed in all preclinical species administered BT1718 at doses above the MTD; in rats (1.5 mg/kg; 9 mg/m² twice weekly), dogs (1 mg/kg; 20 mg/m² twice weekly), and monkeys (2 mg/kg; 24 mg/m² once weekly), significant decreases in WBCs (lymphocytes and neutrophils being the most affected populations) and erythrocyte lineages were observed, correlating with an overall decrease in lymphoid, thymus and bone marrow cellularity. Haematological toxicities such as neutropenia, leucopenia and erythropenia and thrombocytosis are not uncommon in agents that affect the cell cycle, and are clinically manageable.

Renal and Bladder Changes

BT1718, and its possible peptidyl metabolites, are expected to be cleared through the kidney and bladder.

In the rat GLP study renal tubular epithelial degeneration was seen with an onset at 0.2 mg/kg (1.2 mg/m²) twice weekly, incidence and severity increased with dose (minimal to moderate). Moderate renal tubule degeneration and epithelial mitosis was observed at doses above the MTD in non-GLP studies in rats (>1 mg/kg; 6 mg/m² twice weekly), dogs (1 mg/kg; 20 mg/m² twice weekly) and mouse (3.3.5 mg/kg; 10 mg/m² three times weekly). No renal changes were seen in the GLP monkey study in either sex, with the highest dose tested 1.5 mg/kg (18 mg/m²).

The moderate degeneration correlated with an increase in creatinine and urea levels. Tubular basophilia was also seen and is consistent with repair and regeneration [34]. In the rat GLP studies kidney epithelial degeneration showed signs of improvement in the recovery phase, and urea and creatinine levels returned to baseline; recovery was not assessed in the non-GLP studies.

Bladder epithelial degeneration was seen in non-GLP studies in both rat and dog at doses above the MTD (single administration of 40 mg/m² and 14 mg/m², twice weekly respectively). No bladder changes were seen in GLP-compliant studies in either species. In addition, moderate or strong MT1-MMP staining (in 2 of 3 human sections) was observed in the urothelium of the human bladder tissue microarray (TMA) study. However, no membrane staining was seen in the rat or monkey bladder.

Neuronal Changes

Peripheral neuropathy is a common clinical side effect of microtubule inhibitors [35] and antibodies conjugated to DM1. BT1718 caused minimal axonal degeneration in the GLP monkey study with an onset at a dose of 1 mg/kg (12 mg/m²) twice weekly, equivalent to ˜2.4 mg/m² DM1 twice weekly which did not correlate to any obvious neurological deficit (one male of three dosed at 0.5 mg/kg [6 mg/m²] twice weekly, had minimal axonal degeneration). This degeneration was not observed in the rat study and was not evaluated in the dog study. BT1718 has a very different distribution profile to the ADCs. Cantuzumab mertansine a fairly short-lived DM1-conjugated ADC (t_{1/2} hours) showed mild axonal degeneration in animals administered with doses of 58 to 228 mg/m² (0.85 and 3.3 mg/m² DM1) weekly [36] which translated clinically as some neurosensory AEs that were not severe even at the MTD (DLTs of elevated transaminase) [37]. IMGN901 also had a t1/2 of approximately 40 hours and had neurosensory AEs were reported in 17% of all patients (all grades) [38]. Ado-trastuzumab emtansine (T-DM1) which has a t_{1/2} of ˜4 days and therefore a very different distribution in comparison to BT1718, exhibited moderate to severe axonal degeneration when dosed at 120 and 360 mg/m² (2 and 6 mg/m² DM1 content). Schwann cell hyperplasia and hypertrophy and in some cases infiltrating neutrophils also accompanied this T-DM1-mediated axonal degeneration [31, 32, 39] a finding not observed with BT1718. These preclinical findings following administration of T-DM1 translated clinically into 20% of patients experiencing peripheral neuropathy [31, 32, 39].

Hepatic Changes

In the rat GLP 28 day repeat dose study minimal to slight reversible hepatocellular atrophy was observed at 1 mg/kg (6 mg/m²) twice weekly. These findings correlated with reversible raised alkaline phosphatase (ALP) and aspartate aminotransferase (AST) levels and an increase in cholesterol (none of which exceeded 1.5× from control values). Elevated liver enzymes were seen in non-GLP studies in rats (3.35 mg/kg; 20.1 mg/m² twice weekly) and dogs (1 mg/kg; 20 mg/m² twice weekly) with histopathological correlates. No liver toxicity was seen in the GLP-compliant monkey study up to the highest dose tested (1.5 mg/kg; 18 mg/m² twice weekly). Hepatotoxicity has previously been observed with the maytansine toxin (DM1) alone, and antibodies conjugated to DM1, pre-clinically and clinically, however due to the hypothesised altered route of clearance for BT1718 (renal) compared to these maytansinoid compounds (hepatic) patients may be through the kidneys and bladder the liver may be spared from the clinically relevant DLT of transaminitis seen with these other agents.

Local Toxicity (at or Around Injection Site)

In the GLP-compliant monkey study, black colouration of the skin, considered to be due to activation of melanocytes, occurred at areas near the injection sites (and occasionally distant from these sites e.g. at the ankle or fore limbs) in animals dosed at 0.5 mg/kg (6 mg/m²) twice weekly and above, with appearance, distribution and incidence of this finding increasing with increasing dose. This finding was often accompanied by sloughing of the skin/dermatitis near the injection site, which when required, was improved by veterinary invention. Microscopically, these clinical findings correlated with observations of minimal to slight hyperkeratosis and epidermal hyperplasia, along with minimal to slight epidermal pigment. Occasional dermal inflammatory cell infiltrates were seen, but presumed to be a transient inflammatory response, as they were observed only in a subset of animals and not at the highest dose tested (1.5 mg/kg; 18 mg/m² twice weekly). No microscopic skin findings were seen in the recovery animals, though dermal and cutis pigmentation was still evident. The mechanism of the development of the epidermal pigmentation may represent a post-inflammatory hyperpigmentation, which has been reported with a number of chemotherapeutic agents [40]. This local toxicity at the injection site was not observed in any other preclinical toxicology species.

Gastrointestinal Changes

No GI-related findings were observed in the GLP-compliant studies in rat and monkey.

Overt GI tract toxicity were only seen in doses above the MTD in non-GLP studies in mice, rats, dogs and monkeys. Slight to moderate small intestinal degeneration was seen in dog at a dose of 1 mg/kg (20 mg/m²) twice weekly. Single cell necrosis of the ileum was seen in the rat at a dose of 5.03 mg/kg (30.2 mg/m²) twice. In monkeys a single administration of 3 mg/kg (36 mg/m²) one week post 1.5 mg/kg (18 mg/m²) dose, there was mild single cell necrosis of the glandular epithelial cells in the caecum with a mild increase in epithelial cell mitoses in the caecal and colonic mucosa. In mice, intestinal mucosal mitosis was seen at a single dose of 13.4 mg/kg (40.2 mg/m²).

Reproductive Organ Changes

Changes in the reproductive system are a common side effect of agents that affect the cell cycle. In the rat GLP study, all males administered 1 mg/kg (6 mg/m²) twice weekly saw testicular degeneration/atrophy of the seminiferous tubules and the absence of sperm. These changes correlated with a reduction in size and weight of the testes and epididymis. No changes in reproduction organs were seen in monkey studies.

Adrenal Changes

In the rat GLP study adrenal glands had significantly lower terminal weights in males dosed 1 mg/kg (6 mg/m²) twice weekly. Minimal hypertrophy of the zona glomerulosa was also seen in 2 of 10 males administered 0.2 mg/kg (1.2 mg/m²) twice weekly, 3 of 10 males administered 0.5 mg/kg (3 mg/m²) twice weekly and 4 of 10 males administered 1 mg/kg (6 mg/m²) twice weekly, and 5 of 10 females administered 1 mg/kg (6 mg/m²) twice weekly. The changes were minimal and considered to be stress related and therefore may not have a correlation clinically. However, in the TMA study the rat sections (3 of 3) had weak MT1-MMP staining in the zona glomerulosa, indicating they may be target related. Weak to moderate membrane staining was seen in all human sections (3 of 3) in the TMA study. There were no toxicological changes or TMA staining in the monkeys.

Salivary Gland Changes

Dry mouth and associated toxicity are a common side effect with chemotherapy [41], with these symptoms tending to resolve within 3 to 4 weeks off treatment. In the human TMA study 2 of 3 human parotid sections exhibited weak membrane staining for MT1-MMP and therefore the salivary glands could be target of toxicity.

Pancreatic Acinar Changes

The secretory cells of the pancreas had occasional weak to moderate MT1-MMP specific staining on the human pancreatic sections (3 of 3), therefore there is a risk that these cells could be a target for toxicity with BT1718 treatment. There was no expression seen in the rat or the dog TMAs and unsurprisingly no in vivo toxicity findings.

Summary of the Non-Clinical Data

The target organs for BT1718 toxicity in preclinical models have been identified as haematopoietic and lymphoid tissue, kidney and bladder, liver, neuronal, and skin (changes were generally in the vicinity of the injection site) and to a lesser extent other highly replicating tissues such as reproductive organs, GI and secretory cells such as adrenal, pancreatic and salivary tissue. The majority of the toxicities were minimal and reversible. The rat appears to be more sensitive to BT1718 than the other preclinical species tested (mouse, dog and monkey).

In a GLP-compliant study in Cynomolgus monkeys, the HNSTD of BT1718 was established to be 18 mg/m² administered twice weekly, equivalent to a human dose of 0.48 mg/kg. In a GLP-compliant study in the rat, the MTD was not reached with the highest administered dose 6 mg/m² twice weekly. At this dose level, there was kidney toxicity (with no effect on clinical condition) and also testicular toxicity. After 28 days off-dose, there was evidence of partial recovery of kidney-related findings, but no recovery of testicular effects at this dose level. In the non-GLP rat study a dose of 9 mg/m² administered twice weekly was not well tolerated.

Therefore, the MTD for rat for twice weekly dosing was considered to be between 1 and 1.5 mg/kg (6 to 9 mg/m²). Based on the pharmacodynamic studies, the lowest dose producing significant growth inhibition was 3 mg/m² twice weekly (human equivalent dose [HED] of 0.08 mg/kg twice weekly) with 9 mg/m² twice weekly (HED 0.24 mg/kg twice weekly) showing SD or better in several cell-based and PDX tumor models. Similar efficacy was seen in an EBC-1 lung xenograft model when BT1718 was given at 18 mg/m² only once a week. In addition, BT1718 produced SD and regressions in a docetaxel-resistant lung PDX model. Furthermore, BT1718 administered at 30 mg/m² (HED 0.8 mg/kg) twice weekly produced long term complete regressions in a variety of tumor xenograft models. The degree of response to BT1718 in these models correlated with moderate to high expression of MT1-MMP.

Proposed Starting Dose

The HNSTD established in the 28 day-repeat dose GLP monkey study was 1.5 mg/kg (18 mg/m²) twice weekly. Using allometric scaling and applying a standard safety factor of six as noted in the ICH guidance (ICH S9), the human starting dose would be 3 mg/m² twice weekly. In the one month-repeat dose GLP rat study neither the MTD or severe toxicity dose in 10% of the animals (STD₁₀) was established therefore based on highest dose tested, 1 mg/kg (6 mg/m²) twice weekly, with allometric scaling and a standard safety factor of 10 10 (ICH S9), the human starting dose would be 0.6 mg/m² twice weekly (which equates to a DM1 dose of approximately 0.12 mg/m² twice weekly). As the rat is the most sensitive species to BT1718 the starting dose will be based on the highest dose tested in the rat.

Therefore, the proposed starting dose for the FIH Phase I trial is 0.6 mg/m² twice weekly.

Clinical Experience (Phase I Trial (s)/Other Compounds in the Same Class)

No previous clinical studies have been conducted with BT1718. However, maytansine, parent analogue of, DM1 has been evaluated in clinical trials, DM1 has also been evaluated clinically as a component of ADCs, exemplified by T-DM1 which has been licensed by Roche Holding AG under the trade name Kadcyla®. Adverse events experienced with conjugated DM1 are summarized in Table 4 below.

TABLE 4
Summary of adverse events experienced with conjugated DM1

| ADC | Target | Linker | Adverse events and clinical findings |
|---|---|---|---|
| T-DM1 | Anti- | SMCC | In the most recent trial, GATSBY, in HER2 positive |
| (Kadcyla) | HER2 | (Non- | gastric cancer [49, 50] the most common AEs were |
| | | cleavable) | fatigue (33%), anemia (21-33%), neutropenia (10%), |
| | | | thrombocytopenia (26%), GI AEs (25%, made up of |
| | | | constipation, vomiting, diarrhea, nausea, abdominal |
| | | | pain), transaminitis (16%), peripheral neuropathy (10%) |
| | | | and rash (6%). All these AEs were also experienced in |
| | | | breast cancer trials [51]. |
| IMGN529 | anti- | SMCC | In the Phase I trial in relapsed or refractory B-Cell Non- |
| | CD37 | (Non- | Hodgkin Lymphoma (NHL), early onset Grade 3-4 |
| | | cleavable) | neutropenia attributed to cytokine release was reported at |
| | | | 30 mg/m² (0.8 mg/kg). |
| | | | Prophylactic administration of steroids allowed further |
| | | | escalations with the treatment-emergent adverse events |
| | | | (TEAEs in > 20% of the 31 patients)) being neutropenia |
| | | | (30%), fever (27%), asthenia (20%) and fatigue (20%) |
| | | | [52]. IMGN529 is currently recruiting a Phase II study in |
| | | | combination with ritixumab (NCT02564744). |
| AMG595 | anti- | Non- | Phase I in recurrent glioblastoma multiforme and/or |
| | EGFRvIII | cleavable | anaplastic astrocytomas (NCT01475006). Like T-DM1, |
| | | | dose limiting toxicity was thrombocytopenia and has |
| | | | currently been dosed up to 111 mg/m² (3 mg/kg) [53] |
| | | | with full safety data not yet published. |
| Lorvotuzumab | anti- | SPP | A series of Phase I trials have been conducted with |
| mertansine | CD56 | (cleavable) | different schedules in solid malignancies; DLTs included |
| (IMGN901) | | | fatigue, neuropathy, headache or meningitis-like |
| | | | symptoms, chest pain, dyspnea, and myalgias. |
| | | | Recommended Phase 2 dose (RP2D) was 60 mg/m² (3 |
| | | | consecutive days every 3 weeks). Overall, TEAEs were |
| | | | experienced by 96.9% of all patients, the majority of |
| | | | which were Grade 1 or 2. The most commonly reported |
| | | | Grade 3 or 4 TEAEs were hyponatremia and dyspnea |
| | | | (each 8.2%)[38]. Lorvotuzumab mertansine is currently |
| | | | being studied, using a dosing schedule of Day 1 and Day |
| | | | 8 every 21 days, in Phase II trials for hematological |
| | | | malignancies (NCT02420873) and in younger patients |
| | | | with Wilms tumor, rhabdomyosarcoma, neuroblastoma, |
| | | | pleuropulmonary blastoma, malignant peripheral nerve |
| | | | sheath tumor, or synovial sarcoma (NCT02452554). |
| Cantuzumab | Anti- | SPP | Three Phase I trials were conducted in solid tumors; one |
| mertansine | CanAg | (cleavable) | dosing every 3 weeks, another dosing weekly and a third |
| | | | dosing three-times per week in a 3 out of 4 weeks |
| | | | schedule. When administered once every 3 weeks, AEs |
| | | | included acute, transient and reversible elevations of |
| | | | hepatic transaminases, as well as nausea, vomiting, |
| | | | fatigue, and diarrhea (Tolcher et al. 2003), The RP2D |
| | | | from this trial was 235 mg/m² every 3 weeks (~3.9 mg/m² |
| | | | DM1). Weekly administration had a MTD of |
| | | | 115 mg/m²/week (~1.9 mg/m²/week DM1) with DLTs |
| | | | identified as acute, transient elevation of hepatic |
| | | | transaminases and reversible fatigue (Helft et al. 2004), |
| | | | In a third Phase I trial, the ADC was administered IV |
| | | | three-times a week [37]. The MTD was 45 mg/m² |
| | | | (~0.75 mg/m² DM1), and the DLT was Grade 3 |
| | | | transaminitis. Hepatic, hematological, and neurosensory |
| | | | effects also occurred, but were rarely severe, |
| MLN2704 | Anti- | SPP | In the initial Phase I trial evaluating 3 repeat doses at 4 |
| | PSMA | (cleavable) | week intervals the MTD was not determined. MLN2704 |
| | | | was given up to 343 mg/m²/week per dose (ca. 5.7 mg/m² |
| | | | DM1). Drug-related Grade 3 toxicities occurred in three |
| | | | (13%) of 23 patients, including uncomplicated febrile |
| | | | neutropenia (the only DLT) in one patient, reversible |
| | | | elevations in hepatic transaminases, leukopenia, and |
| | | | lymphopenia. The most frequent Grade 1 or 2 toxicides |
| | | | included fatigue, nausea, and diarrhea. Neuropathy |
| | | | occurred in eight (35%) of 23 patients, including five of |
| | | | six patients treated at 343 mg/m² [54]. In an additional |
| | | | Phase I trial, which explored multiple schedules, |
| | | | neurotoxicity was dose-limiting, with 44 of 62 patients |
| | | | exhibiting peripheral neuropathy, including 6 having |
| | | | Grade 3/4 neuropathy [55]. Other common toxicides |
| | | | included nausea (61%), fatigue (60%), anorexia (39%), |
| | | | and diarrhea (39%). Overall, 15 patients (38%) |
| | | | discontinued treatment secondary to an adverse event. |

Expected Safety Profile for BT1718

Hematopoietic Changes

Changes in haematology parameters were observed in rats, dogs and monkey administered BT1718. Haematological toxicity such as neutropenia, lymphopenia, erythropenia and thrombocytosis are not uncommon in agents that affect the cell cycle and these changes are clinically manageable. Minimal lymphopenia, erythropenia and thrombocytosis was seen for BT1718 in the GLP studies, with histopathological correlates observed in non-GLP studies at doses above the MTD. BT1718 is likely to produce hematological changes in patients and as such, standard hematological evaluations will be undertaken as part of the clinical programme.

Renal and Bladder Changes

BT1718 and its possible peptidyl metabolites, are expected to be cleared through the kidney and bladder. In preclinical species, renal tubular epithelium degeneration was seen in rats and was dose dependent in severity and incidence. Increases in creatinine and urea levels were also observed in animals, generally at doses at or above the MTD. Tubular basophilia was also found and is consistent with repair and regeneration [34]. Bladder epithelial degeneration was seen in non-GLP studies in both rat and dog (40 mg/m² and 14 mg/m² respectively) above the later determined MTD. In addition, urothelium of the bladder had moderate or strong MT1-MMP staining in the TMA studies conducted (2 of 3 human sections).

Hepatic Changes

Minimal to slight reversible hepatocellular atrophy, with correlating small increases in ALP, AST and cholesterol levels, was observed in the rat. Similar findings were noted in the dog at high doses, but not in the monkey. Hepatotoxicity has been a common finding for the predominantly hepatic-cleared maytansine and antibody-maytansinoid conjugates, but may be minimal for BT1718 given the differential renal clearance pathway for this maytansinoid conjugate. However, as a precaution standard liver function tests will be performed as part of the clinical programme. Further investigations or imaging would be initiated as clinically indicated.

Neuronal Changes

Peripheral neuropathy is a common side effect of microtubule inhibitors [35] and has been a noted AE experienced with antibody-DM1 conjugates. BT1718 caused minimal axonal degeneration in the monkey without any obvious neurological clinical findings. The altered bio distribution and limited systemic exposure may reduce the incidence of clinical neuropathies. Patients will be evaluated at study visits for any AEs relating to the nervous system. Symptom-directed clinical examination and further investigations or imaging would be initiated as clinically indicated. Further investigations or imaging would be initiated as clinically indicated.

Skin

Dose-related findings in the skin including sloughing, dermatitis and increased pigmentation were observed in the monkey. Patients will be evaluated at study visits for any AEs related to the skin. In addition, DM1 and other chemotherapeutics can cause extravasation and/or are vesicants, irritants, inflammitants or exfoliants [56, 57] and as a precaution BT1718 will be treated as a vesicant. Patients will be evaluated during treatment and at each study visit for evidence of extravasation. Standard local policies for management of vesicant extravasation will be followed, typically starting with stopping the infusion, aspirating if possible, topical hydrocortisone and ongoing review. The role of specific treatment such as heat or cold packs, dimethyl sulfoxide (DMSO) or hyaluronidase is unknown.

Gastrointestinal Changes

Gastrointestinal effects were not seen in animals with BT1718 at doses below their MTDs in the GLP studies, with degeneration and apoptotic histopathologies noted at high doses in non-GLP studies. Gastrointestinal toxicity is therefore considered unlikely below the MTD in humans. Patients will be evaluated at study visits for any AEs relating to the GI tract (see pancreatic acinar section below). Further investigations or imaging may be initiated if clinically indicated.

Reproductive Organ Changes

Changes in the reproductive system are a common side effect of agents that affect the cell cycle. In the rat GLP study all males administered 1 mg/kg (6 mg/m²) twice weekly had testicular degeneration/atrophy of the seminiferous tubules and absence of sperm. No changes in reproduction organs were seen in monkey studies.

Reproductive changes may be a possible side effect of administration of BT1718. Patients will be advised of the potential impact of BT1718 on fertility and patients will be required to comply with the standard clinical trial contraceptive practices. The Phase I trial will be in refractory/relapsed patients whose prognosis and fertility are likely to be very limited, therefore in this context it is deemed an acceptable risk. Where appropriate for male patients who may be in a position to consider having or extending a family, the possibility of conservation of sperm should be discussed. Patients wishing to do so should also have a discussion of the implications of their own prognosis and of the possible effects of previous therapy on the production, function and genetic health of sperm.

Adrenal Changes

Minimal hypertrophy of the zona glomerulosa was seen in the rat GLP study in subset of animals at each dose. Additionally, a rat TMA study conducted saw 3 of 3 sections had weak MT1-MMP staining in zona glomerulosa and in human TMA sections weak to moderate staining was also noted in 3 of 3 sections. Administration of BT1718 could lead to changes in adrenal gland function including the zona glomerulosa (aldosterone secretion). As such, BP and standard clinical chemistry parameters will be evaluated as part of the clinical programme. Persistent unexplained hypo-/hyper-tension or altered potassium levels would be investigated further with serum renin:aldosterone, cortisol or ACTH assays as appropriate, and treatment such as fluids and steroid replacement, or conversely anti-hypertensives, initiated as clinically indicated.

Salivary Gland Changes

Dry mouth and associated toxicity are a common side effect with chemotherapy [41], with these symptoms tending to resolve within 3 to 4 weeks off treatment. In the human TMA study 2 of 3 human parotid sections exhibited weak membrane staining for MT1-MMP and therefore the salivary glands could be target of toxicity.

Patients will be evaluated at study visits for any AEs, including dry mouth. Symptom-directed clinical examination/and or further investigations will be conducted as clinically indicated.

Pancreatic Acinar Changes

The secretory cells of the pancreas had weak to moderate MT1-MMP specific staining on the three TMAs conducted therefore there is a risk that these cells could be a target for toxicity with BT1718 treatment. There was no expression seen in the rat or the dog TMAs and no in vivo MT1-MMP toxicity findings. Toxicity might be expected to manifest as impaired exocrine function of the pancreas. Patients will be evaluated at study visits for any AEs relating to the GI tract (see also gastrointestinal section above). Bloating, steatorrhea and/or diarrhea may be investigated further with faecal elastase evaluation and treatment such as CREON initiated as clinically indicated.

Rationale for the Proposed Trial

By targeting MT1-MMP expressing tumor cells, BT1718 is expected to induce selective tumor cell death. This would be expected to translate into objective radiological responses with an acceptable therapeutic window, and ultimately to improve PFS and OS for patients with MT1-MMP expressing tumors. Preclinical data has demonstrated activity in relevant models and toxicology has indicated monitorable and reversible toxicities, expected to be manageable in the clinic.

BT1718 has low molecular weight (3.5 kDA) in comparison to other conjugated toxin approaches, which enables rapid penetration of tumor tissue. In addition, preclinical PK and toxicokinetics estimates a 15 to 30 minute half-life, which is in contra-distinction to ADCs. Hypothesized advantages over ADCs therefore include reduced systemic exposure of normal tissues to circulating BT1718, the ability to manage toxicity during recovery periods, as well as the improved tumor penetrance. Other potential advantages include a fixed peptide:conjugate ratio of 1:1 (c.f. ADCs where variable conjugation results in mixed populations) and with more scalable manufacturing as a small molecule (c.f. biologics such as ADCs).

Overexpression of MT1-MMP has been reported in NSCLC [3, 4], breast cancer [5, 6, 58] and other solid tumors [7, 8, 9]. Work is ongoing in identifying those tumor types with the highest incidence of MT1-MMP overexpression. BT1718 anti-tumor activity was generally higher in xenograft models that had high levels of MT1-MMP expression, though anti-tumor activity was observed in some models with low MT1-MMP expression. Since the majority of cancers may express some MT1-MMP, and the relationship between efficacy and MT1-MMP expression is not fully delineated the dose escalation phase of the trial will not restrict recruitment based on levels of MT1-MMP expression, and will be open to patients of all solid malignancy types. In the Phase IIa, expansion phase at the optimal dose/schedule(s), patients will be enrolled with tumor types anticipated to commonly overexpress MT1-MMP and where raised high MT1-MMP overexpression is confirmed during prospective screening selection at enrolment. This confirmation of expression will test the hypothesis that MT1-MMP overexpression is expected to translate to favorable clinical outcomes for patients treated with BT1718. These tumor types are currently proposed for the Phase IIa to include NSCLC, TNBC, ovarian, sarcomas, and tumor types expressing MT1.

The route of administration will be intravenous. All preclinical efficacy and toxicity studies have been intravenous to support this method of delivery. Initially, twice weekly dosing will be evaluated as supported by preclinical efficacy and toxicity studies. Due to the PK of BT1718, frequent dose intervals may allow a higher dose density, and safety and clinical PKs can be directly correlated to the preclinical species. During the escalation phase, a once weekly regimen will also be explored, which is expected to be more convenient for patients and preliminary preclinical data suggests may have similar activity.

### Additional Embodiments

### Inclusion Criteria

As described herein, patients must fulfil the eligibility criteria listed in Table 8. In some embodiments, a patient fulfils the eligibility criteria listed in Table 8.

### Exclusion Criteria

As described herein, patients will be excluded if they meet any of the criteria as listed in Table 9. In some embodiments, an excluded patient meets a criterion in Table 9. Dose

As described herein, BT1718 is administered at a dose of about 0.3 mg/m² to about 45 mg/m². In some embodiments, BT1718 is administered at a dose of about 0.3 mg/m² to about 45 mg/m². In some embodiments, BT1718 is administered at a dose of about 0.3 mg/m² to about 45 mg/m², or a fraction thereof. In some embodiments, BT1718 is administered at a dose of about 0.5 mg/m². In some embodiments, BT1718 is administered at a dose of about 1 mg/m². In some embodiments, BT1718 is administered at a dose of about 2 mg/m². In some embodiments, BT1718 is administered at a dose of about 3 mg/m². In some embodiments, BT1718 is administered at a dose of about 4 mg/m². In some embodiments, BT1718 is administered at a dose of about 5 mg/m². In some embodiments, BT1718 is administered at a dose of about 6 mg/m². In some embodiments, BT1718 is administered at a dose of about 7 mg/m². In some embodiments, BT1718 is administered at a dose of about 8 mg/m². In some embodiments, BT1718 is administered at a dose of about 9 mg/m². In some embodiments, BT1718 is administered at a dose of about 10 mg/m².

In some embodiments, BT1718 is administered at a dose of about 11 mg/m². In some embodiments, BT1718 is administered at a dose of about 12 mg/m². In some embodiments, BT1718 is administered at a dose of about 13 mg/m². In some embodiments, BT1718 is administered at a dose of about 14 mg/m². In some embodiments, BT1718 is administered at a dose of about 15 mg/m². In some embodiments, BT1718 is administered at a dose of about 16 mg/m². In some embodiments, BT1718 is administered at a dose of about 17 mg/m². In some embodiments, BT1718 is administered at a dose of about 18 mg/m². In some embodiments, BT1718 is administered at a dose of about 19 mg/m². In some embodiments, BT1718 is administered at a dose of about 20 mg/m².

In some embodiments, BT1718 is administered at a dose of about 21 mg/m². In some embodiments, BT1718 is administered at a dose of about 22 mg/m². In some embodiments, BT1718 is administered at a dose of about 23 mg/m². In some embodiments, BT1718 is administered at a dose of about 24 mg/m². In some embodiments, BT1718 is administered at a dose of about 25 mg/m². In some embodiments, BT1718 is administered at a dose of about 26 mg/m². In some embodiments, BT1718 is administered at a dose of about 27 mg/m². In some embodiments, BT1718 is administered at a dose of about 28 mg/m². In some embodiments, BT1718 is administered at a dose of about 29 mg/m². In some embodiments, BT1718 is administered at a dose of about 30 mg/m².

In some embodiments, BT1718 is administered at a dose of about 31 mg/m². In some embodiments, BT1718 is administered at a dose of about 32 mg/m². In some embodiments, BT1718 is administered at a dose of about 33 mg/m². In some embodiments, BT1718 is administered at a dose of about 34 mg/m². In some embodiments, BT1718 is administered at a dose of about 35 mg/m². In some embodiments, BT1718 is administered at a dose of about 36 mg/m². In some embodiments, BT1718 is administered at a dose of about 37 mg/m². In some embodiments, BT1718 is administered at a dose of about 38 mg/m². In some embodiments, BT1718 is administered at a dose of about 39 mg/m². In some embodiments, BT1718 is administered at a dose of about 40 mg/m².

In some embodiments, BT1718 is administered at a dose of about 41 mg/m². In some embodiments, BT1718 is administered at a dose of about 42 mg/m². In some embodiments, BT1718 is administered at a dose of about 43 mg/m². In some embodiments, BT1718 is administered at a dose of about 44 mg/m². In some embodiments, BT1718 is administered at a dose of about 45 mg/m².

### Route of Administration

As described herein, BT1718 is administered intravenously.

### Dosing Schedule

As described herein, BT1718 is administered intravenously once weekly for three out of four weeks. As described herein, BT1718 is administered intravenously twice weekly for three out of four weeks.

In some embodiments, BT1718 is administered intravenously twice weekly at a dose of about 0.6-9.6 mg/m². In some embodiments, BT1718 is administered intravenously twice weekly at a dose of about 0.6 mg/m². In some embodiments, BT1718 is administered intravenously twice weekly at a dose of about 1.2 mg/m². In some embodiments, BT1718 is administered intravenously twice weekly at a dose of about 2.4 mg/m². In some embodiments, BT1718 is administered intravenously twice weekly at a dose of about 4.8 mg/m². In some embodiments, BT1718 is administered intravenously twice weekly at a dose of about 7.2 mg/m². In some embodiments, BT1718 is administered intravenously twice weekly at a dose of about 9.6 mg/m².

In some embodiments, BT1718 is administered by an infusion of about 1 hour at each administration. In some embodiments, BT1718 is administered intravenously twice weekly at an infusion rate of about 0.015-0.245 mg/kg/h. In some embodiments, BT1718 is administered intravenously twice weekly at an infusion rate of about 0.015 mg/kg/h. In some embodiments, BT1718 is administered intravenously twice weekly at an infusion rate of about 0.031 mg/kg/h. In some embodiments, BT1718 is administered intravenously twice weekly at an infusion rate of about 0.061 mg/kg/h. In some embodiments, BT1718 is administered intravenously twice weekly at an infusion rate of about 0.123 mg/kg/h. In some embodiments, BT1718 is administered intravenously twice weekly at an infusion rate of about 0.184 mg/kg/h. In some embodiments, BT1718 is administered intravenously twice weekly at an infusion rate of about 0.245 mg/kg/h.

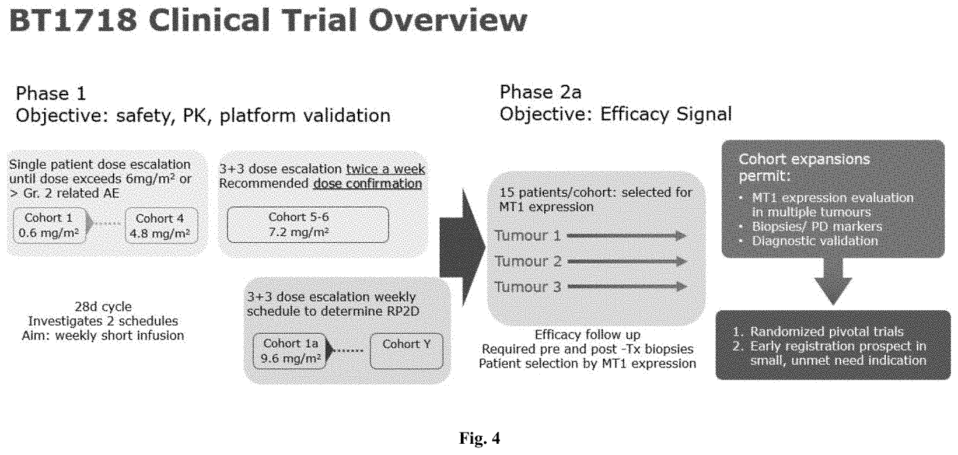

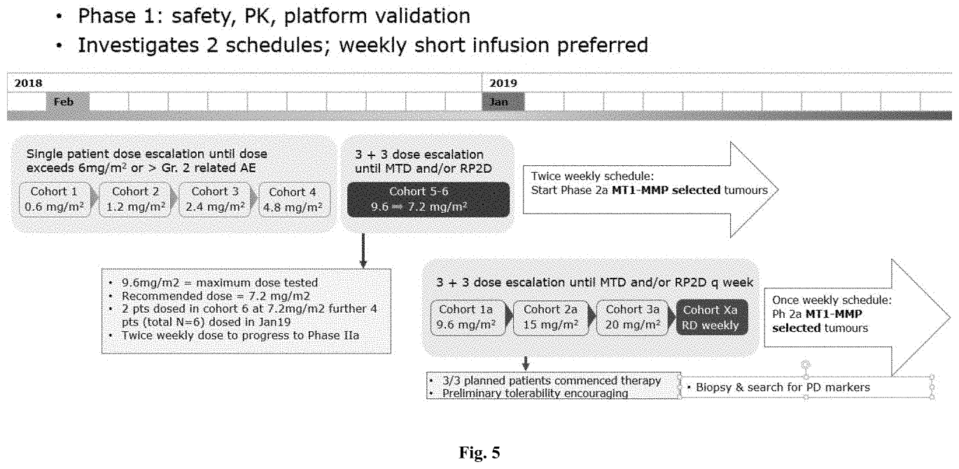

In some embodiments, BT1718 is administered at a dose and schedule as described in FIGS. 4 and 5.

### Pharmacokinetic Data

In some embodiments, CLp is about 10 mL/min/kg to 12 mL/min/kg. In some embodiments, CLp is about 10 mL/min/kg. In some embodiments, CLp is about 12 mL/min/kg. In some embodiments, CLp is about 11 mL/min/kg.

In some embodiments, t½ is about 10 min to 20 min. In some embodiments, t½ is about 10 min. In some embodiments, t½ is about 11 min. In some embodiments, t½ is about 12 min. In some embodiments, t½ is about 13 min. In some embodiments, t½ is about 14 min. In some embodiments, t½ is about 15 min. In some embodiments, t½ is about 16 min. In some embodiments, t½ is about 17 min. In some embodiments, t½ is about 18 min. In some embodiments, t½ is about 19 min. In some embodiments, t½ is about 20 min.

Pharmaceutically Acceptable Compositions

According to another embodiment, the invention provides a composition comprising BT1718, or a pharmaceutically acceptable salt thereof, and a pharmaceutically acceptable carrier, adjuvant, or vehicle.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

The term “subject,” as used herein, is used interchangeably with the term “patient” and means an animal, preferably a mammal. In some embodiments, a subject or patient is a human. In other embodiments, a subject (or patient) is a veterinary subject (or patient). In some embodiments, a veterinary subject (or patient) is a canine, a feline, or an equine subject.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

In certain embodiments, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

Pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. In certain embodiments, the compounds of the invention may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of BT1718, it may be desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

BT1718, or a pharmaceutically acceptable salt thereof, can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

### EXEMPLIFICATION

The following Examples illustrate the invention described above; they are not, however, intended to limit the scope of the invention in any way. The beneficial effects of the pharmaceutical compounds, combinations, and compositions of the present invention can also be determined by other test models known as such to the person skilled in the pertinent art.

### Example 1

### A Phase I/IIa Trial of BT1718 Given Intravenously in Patients with Advanced Solid Tumors

### Trial Design

### Clinical Trial Objectives and Endpoints

### Primary Objectives and Endpoints

The primary objectives and endpoints are provided in Table 5, below.

TABLE 5
Primary Objectives and Endpoints.

| Primary objectives | Endpoints |
|---|---|
| Dose Escalation phase | |
| To propose a recommended Phase II dose | Determine a dose at which no more than one |
| (RP2D) for evaluation by establishing the | out of six patients at the same dose level |
| maximum tolerated dose (MTD) and/or | experiences a probable or highly probable |
| maximum administered dose (MAD), of | BT1718-related DLT. |
| BT1718 given in patients with advanced solid | |
| tumors, at one or more dosing schedules. | |
| Dose escalation and expansion phase | |
| To assess the safety and toxicity profile of | Determine the frequency and causality of each |
| BT1718 in patients with advanced solid | AEs to BT1718 and grade severity according to |
| tumors. | NCI CTCAE Version 4.02. |
| | The causality of all AEs will be assessed by the |
| | Investigator. |

The secondary objectives and endpoints are provided in Table 6, below.

TABLE 6
Secondary Objectives and Endpoints.

| Secondary objectives | Endpoints |
|---|---|
| Dose escalation phase | |
| To investigate the pharmacokinetic (PK) | Measurement of Cₘₐₓ, AUC, terminal |
| behavior of BT1718 in human | elimination half-life (t½), and other PK |
| | parameters of BT1718 in plasma, both as an |
| | intact and cleaved molecule. |
| Dose escalation and expansion phase | |
| To assess preliminary signals of BT1718 | Assess anti-tumor response according to |
| efficacy, including in relevant tumor types with | Response Evaluation Criteria in Solid Tumors |
| high expression of membrane type 1 matrix | (RECIST) Version 1.1 using computerized |
| metalloproteinase (MT1-MMP) in Phase IIa | tomography (CT) or magnetic resonance |
| | imaging (MRI) scans. |
| | Estimate progression-free survival, |
| | progression-free survival rate at six months, |
| | and OS (where available). |
| | Estimate duration of response. |

The tertiary objectives and endpoints are provided in Table 7, below.

TABLE 7
Tertiary Objectives and Endpoints.

| Tertiary objectives | Endpoints |
|---|---|
| Dose escalation and expansion phase | |
| To explore potential predictive and | Assess MT1-MMP status by IHC, in tumor |
| pharmacodynamic biomarkers of BT1718 | tissues, stroma as well as other potential |
| pre and post BT1718 treatment in tumor | exploratory pharmacodynamic biomarkers and |
| tissue and stroma | DM1, collected at baseline and pre and post |
| | BT1718 treatment. |
| | Heterogeneity of those biomarkers (where |
| | multiple samples are available over site/time), |
| | comparison to non-tumor tissues (where |
| | available) and correlation between archival |
| | and fresh tissue (where available). |
| Dose escalation and expansion phase | |
| To investigate immunogenicity of | To evaluate anti drug antibodies (ADA) |
| BT1718in blood | against BT1718. |
| Dose escalation and expansion phase | |
| To explore cell free DNA (cfDNA) levels | Assess cfDNA levels following treatment and |
| in response to BT1718 in blood | use including but not limited to, as a marker of |
| | surrogate efficacy. |
| Dose escalation and expansion phase | |
| To investigate circulating cell death | Measure M30 and M65 at a range of time |
| biomarkers in response to BT1718 in | points pre and post BT1718 treatment. |
| blood | |
| Dose escalation phase | |
| To investigate the total DM1-SH and | Measurement of total DM1-SH as a measure |
| potentially the intact molecule in urine | of DM1 in BT1718, any peptidyl-DM1 |
| | metabolites of BT1718, other DM1-containing |
| | mixed disulfides and free DMI in urine |
| | samples. In addition, intact BT1718 may also |
| | be analyzed. |
| Expansion phase | |
| To explore the pharmacodynamic effects | Measure markers of cell death in tumor, |
| of BT1718 on tissue and/or blood | stromal, immune +/− healthy tissue, and |
| samples, including potential resistance | correlation with MT1-MMP status, including |
| biomarkers | bystander effects, may be assessed pre and |
| | post treatment. |
| | Potential resistance biomarkers. |
| Expansion phase | |
| To explore potential correlation between | Examine potential correlations between the |
| pharmacodynamic, toxicity and clinical | AE profile, pharmacodynamic and/or tumor |
| activity in patients | response. |

Design of the Clinical Trial

This is a multi-center, FIH, Phase I/IIa, open label dose escalation trial with an expansion phase, in patients with advanced solid tumors.

This clinical trial will consist of two phases, Phase I and Phase IIa (FIG. 4).

Phase I, Dose Escalation Phase:

Phase I Will Consist of Stage 1 and Stage 2. Stage 2 May Commence Before Stage 1 is completed:

Stage 1—BT1718 will be administered intravenously twice weekly for three out of four weeks until the RP2D and/or MTD is established.

Single patient cohorts 1, 2, 3, and 4 of the twice-weekly schedule completed without significant toxicity, reaching target threshold and triggering 3+3 escalation. Cohort 5 (9.6 mg/m²) completed with 2×DLTs (GGT, fatigue) and a 7.2 mg/m² cohort has been opened. Once weekly escalation is also now open (9.6 mg/m²).

Stage 2—BT1718 will be administered intravenously once weekly for three out of four weeks until the RP2D and/or MTD is established.

Stage 2 will open at a dose where there is expectation of potential biological activity based on available toxicity, PK and/or PD data from Stage 1. This stage will follow a 3+3 dose escalation design and will include a minimum of three evaluable patients at each dose level. Dose increases will be up to 100%, driven by reported safety and available PK data.

Phase IIa, Expansion Phase

In the Phase IIa, expansions at the optimal dose/schedule(s), tolerability will be further characterised and a clinical signal will be explored in an enriched population with tumor types known to commonly over-express MT1-MMP and where MT1-MMP overexpression is confirmed during prospective selection at enrolment. These tumor types are currently proposed to include NSCLC, TNBC or Sarcoma.

Part A—BT1718 will be administered intravenously at the twice weekly MTD and/or RP2D established in Phase I, Stage 1 and will include 14 patients, comprised of equal numbers of patients from two indications with MT1-MMP positive tumors currently proposed to be NSCLC and TNBC. At least six patients will have paired tumor biopsies prior to treatment and while on treatment. Phase IIa, Part A (twice weekly expansion) may run in parallel with Phase I, Stage 2 (once weekly dose escalation).

Part B—BT1718 will be administered intravenously at the once weekly MTD and/or RP2D established in Phase I Stage 2 and will include 14 patients, comprised of equal numbers of patients with MT1-MMP positive tumors currently proposed to be NSCLC and TNBC. At least six patients will have paired tumor biopsies prior to treatment and while on treatment. Phase IIa, Part B (once weekly expansion) may run in parallel with Phase I, Stage 1 (twice weekly dose escalation).

Part C & Part D—following completion of Parts A and B, results will be reviewed by the Joint Development Committee, compromising of team members from the Sponsor, BicycleRD and the CI. Following review of the data generated to date in Phase I (Stages 1 and 2) and Phase IIa (Parts A and B), a decision will be made about which dosing scheme to employ (once or twice weekly), the starting dose and in which tumor indications. Following this decision, up to two additional cohorts of approximately 15-16 patients each will be enrolled in Parts C and D, respectively, with patient populations chosen based on pre-clinical and clinically emerging data. These tumor types are currently proposed to include NSCLC, TNBC or Sarcoma.

In Phase I, Stages 1 and 2, it is expected between 50 to 60 patients with advanced solid tumors, whose tumors have progressed through any suitable standard therapies, will be entered into this study. The final number of patients will depend on the number of dose escalations required to identify the MTD and/or RP2D, at one or more dosing schedules.

Alternative dosing schedules may be considered by the Sponsor based on emerging data during the study, for example if the toxicology profile is benign with the twice weekly dosing regimen, continuous bi-weekly dosing may be evaluated. Emerging data obtained during Phase I may be used in the decision to proceed with the Phase IIa stages. In addition, depending on the patient populations selected, an increased dosing frequency may also be considered. If any changes are made to the dosing schedule a substantial amendment will be submitted to the MHRA, REC and HRA for approval. In Phase IIa, Parts A, B, C and D, it is expected that up to an additional 60-70 patients will be evaluated in up to three tumor types, to be identified based on pre-clinical and clinical data, currently proposed to be NSCLC, TNBC and sarcoma.

In Phase I, Stage 1, BT1718 will be administered intravenously twice weekly for three out of four weeks (dosing on Days 1, 4, 8, 11, 15 and 18). The starting dose will be 0.6 mg/m² and each cycle will last 28 days. Patients may continue treatment until disease progression (depending on the availability of BT1718).

In Phase I, Stage 2, BT1718 will be administered intravenously once weekly for three out of four weeks (dosing on Days 1, 8 and 15). The starting dose of the once weekly regime will be up to 100% of the overall weekly dose from the last completed cohort deemed safe from the twice weekly schedule (Phase I, Stage 1). Patients may continue treatment until disease progression (depending on the availability of BT1718).

Definition of Dose Limiting Toxicity

Some of the DLT and MTD definitions are derived from the NCI CTCAE Version 4.02. Please note that not all of the events described as DLTs are fully supported by NCI CTCAE but are formed by amalgams of different events in order to assist with assessments of AEs.

A DLT is defined as a probably or highly probably drug-related AE occurring during Cycle 1 (i.e. the first 28 days) which fulfils one or more of the following criteria, despite appropriate supportive clinical management (however, all clinically significant toxicities will be considered in dose review decisions and the determination of the Phase II dose):
Neutropenia Grade 4 (absolute neutrophil count [ANC]<0.5×10⁹/L) for ≥seven days
see note;
Febrile neutropenia with Grade 3 or 4 neutropenia ANC<1.0×10⁹/L and a single temperature of ≥38.3° C. or a sustained temperature ≥38° C. for more than one hour);
Clinically significant infection (documented clinically or microbiologically) with Grade 3 or 4 neutropenia (ANC<1.0×10⁹);
Thrombocytopenia Grade 4:
a) for ≥five days *see note, or
b) associated with active bleeding, or
c) requiring platelet transfusion.
   Grade 3 or 4 toxicity to organs other than the bone marrow, EXCLUDING:
   Grade 3 nausea;
   Grade 3 or 4 vomiting in patients who have not received optimal treatment with anti-emetics; or
   Grade 3 or 4 diarrhoea in patients who have not received optimal treatment with anti-diarrhoeals.
   Transient, asymptomatic Grade 3 biochemical abnormalities if agreed by the Sponsor and the Study Team, including the Chief Investigator (CI).
   Fatal event;
   Any other related toxicity which leads to discontinuation of treatment in Cycle 1, or to a delay in dosing of >7 days (excluding where the delay is >7 days due to scheduling rather than a clinical decision);
   Any other related toxicity that is greater than at baseline and is judged to be a DLT by the Sponsor, including the CI and Principal Investigators (PIs).

*Note: In the event of a Grade 4 neutropenia or Grade 4 thrombocytopenia, a full blood count must be performed at least on Day 7 (neutropenia) and Day 5 (thrombocytopenia) after the onset of the event to determine if a DLT has occurred. The investigator must continue to monitor the patient closely until resolution to Grade 3 or less.

Dose limiting toxicities defined above will be considered for the purpose of dose escalation decisions; however, should cumulative toxicity become apparent this will also be taken into consideration when determining either the next dose level or the RP2D.

Definition of Maximum Tolerated Dose

If one instance of DLT as defined in (dose limiting section above) is observed in a cohort of three patients, up to a total of six patients will be treated at that dose level. If one out of six patients experiences a DLT, dose escalation will continue. If two or more out of up to six patients experience a DLT, dose escalation will stop and this dose will be defined as non-tolerated. A maximum of six evaluable patients will be treated at a dose below the non-tolerated level, to define the MTD.

The MAD will be defined as the highest dose received. This will usually represent the non-tolerated dose above the MTD, but may instead represent a maximum administrable dose if the feasible volume of infusion limits dose before toxicity does.

The RP2D for the expansion phases (Parts A and B) for both once weekly and twice weekly dosing will be determined following discussion of all clinically relevant toxicity, efficacy data, and PK results data by the CI, PIs and the Sponsor's Medical Advisor. All significant toxicities will be considered in the determination of the RP2D, including all available data from all cycles and cohorts of treatment.

Patient Evaluability

Response

All patients who meet the eligibility criteria and receive ≥75% of the planned dose exposure of BT1718 and have a baseline assessment of disease and at least one repeat disease assessment will be evaluable for response.

Repeat assessments after at least four weeks are required in order for a patient to be confirmed as having a complete or partial response (CR or PR). To be assigned a status of SD, follow-up measurements must have met the SD criteria at least once and at least six weeks after the initial dose of BT1718 is given.

Safety

All patients who meet the eligibility criteria and receive at least one administration of BT1718 will be evaluable for safety.

Dose Escalation

In the Phase I, Stage I, single patient dose escalation phase of this study, each patient must have received all of their planned doses of BT1718 during the first cycle (28 day DLT period) to make a decision to escalate to the next single patient cohort. If a patient does not receive all their planned doses of BT1718 during that cycle for reasons other than toxicity, a further evaluable patient may need to be recruited before a decision can be made.

Once the dose escalation cohorts are expanded following a 3+3 design, patients must have received ≥75% of their planned dose exposure of BT1718 during the first cycle (28 day DLT period) in order to dose escalate. If any patients in the three to six patient cohorts receive <75% of their planned doses during the first cycle (28 day DLT period) for reasons other than BT1718 related toxicity, further evaluable patients may need to be recruited before a decision can be made.

Patient Selection

Eligibility Criteria

The patient must fulfil the eligibility criteria listed in Table 8 below:

TABLE 8
Inclusion criteria.

| No. | Description | |
|---|---|---|
| 1. | Written (signed and dated) informed consent and be capable of co-operating with | |
| | treatment and follow-up | |
| 2. | Phase I, dose escalation phase (Stages 1 and 2): | |
| | Histologically or cytologically proven advanced solid tumor, refractory to | |
| | conventional treatment, or for which no conventional therapy is considered | |
| | appropriate by the Investigator or is declined by the patient. | |
| | Phase IIa, expansion phase (Parts A, B, C and D): | |
| | Histologically or cytologically proven advanced solid tumor of particular | |
| | interest based on pre-clinical and clinical data, refractory to conventional | |
| | treatment, or for which no conventional therapy is considered appropriate by | |
| | the Investigator or is declined by the patient. For Part A and Part B, this is | |
| | currently proposed to be NSCLC or TNBC (with seven NSCLC and seven | |
| | TNBC in each cohort). | |
| | At least one measurable lesion according to RECIST criteria Version 1.1, that | |
| | has had objective radiological progression on or after the last therapy | |
| | High MT1-MMP expression by IHC assay of archival tumor sample | |
| | (mandatory fresh tumor samples for those patients without available archival | |
| | tumor samples). | |
| | Consent for paired fresh tumor sample at baseline and on treatment in a | |
| | minimum of six patients. | |
| | Consent for non-tumor sample at baseline and on treatment (optional) for | |
| | patients having a paired tumor biopsy. | |
| 3. | Life expectancy of at least 12 weeks | |
| 4. | World Health Organization (WHO) performance status of 0-1 (Appendix 1) | |
| 5. | Haematological and biochemical indices within the ranges shown below. These | |
| | measurements should be performed to confirm the patient’s eligibility. | |
| | Laboratory Test | Value required |
| | Haemoglobin (Hb) | ≥9.0 g/L, or ≥10.0 g/L if transfusion within last |
| | | four weeks |
| | Absolute neutrophil count | ≥1.5 × 10⁹/L |
| | (ANC) | |
| | Platelet count | ≥100 × 10⁹/L |
| | Bilirubin | ≤1.5 × upper limit of normal (ULN) |
| | Alanine amino-transferase | ≤2.5 × ULN (or ≤5 × ULN if has liver |
| | (ALT), aspartate amino- | metastases) |
| | transferase (AST) and alkaline | |
| | phosphatase (ALP) | |
| | Renal function | ≤1.5 × ULN |
| | Either: | |
| | Serum creatinine | |
| | Or: | GFR ≥ 50 mL/min (uncorrected value) |
| | Calculated creatinine clearance | |
| | (using the Wright or Cockcroft & | |
| | Gault [C&G] formula) | |
| | Or: | GFR ≥ 50 mL/min (corrected value) |
| | Isotope clearance measurement * | |

| | * Isotope clearance result to be used to confirm eligibility if calculated C&G/Wright |
|---|---|
| | method results in a glomerular filtration rate (GFR) of = 50 mL/min. |
| 6. | 16 years or over at the time consent is given |
| 7. | Consent to access and analyse any available archival tissue. |

Exclusion Criteria

Exclusion criteria are provided in Table 9 below.

TABLE 9
Exclusion Criteria.

| No. | Description |
|---|---|
| 1. | Radiotherapy (except for palliative reasons), endocrine therapy, chemotherapy or |
| | investigational medicinal products during the previous four weeks (six weeks for |
| | nitrosoureas, mitomycin-C) before treatment (or first dose of an immunotherapy |
| | during the previous 12 weeks). |
| 2. | Prior bone marrow transplant, myeloablative conditioning, or extensive radiotherapy |
| | to greater than 25% of bone marrow, within previous eight weeks of first BT1718 |
| | dose. |
| 3. | Ongoing toxic manifestations of previous treatments greater than NCI CTCAE Grade |
| | 1. Exceptions to this are alopecia, amenorrhea/oligospermia and any other ongoing |
| | toxic manifestation which in the opinion of the Investigator and the Medical Advisor |
| | should not exclude the patient. |
| 4. | Any CNS metastases (unless had local therapy and are asymptomatic and |
| | radiologically-stable off steroids for the last four weeks). |
| 5. | Current or prior malignancy which could affect compliance with the protocol or |
| | interpretation of results. Patients with curatively-treated non-melanoma skin cancer, |
| | non-muscle-invasive bladder cancer, or carcinomas-in-situ are generally eligible. |
| 6. | Female patients who are able to become pregnant (or are already pregnant or |
| | lactating). However, those patients who have a negative serum or urine pregnancy test |
| | before enrolment and agree to use two forms of contraception (one effective form plus |
| | a barrier method) [oral, injected or implanted hormonal contraception and condom; |
| | intra-uterine device and condom; diaphragm with spermicidal gel and condom] or |
| | agree to sexual abstinence*, effective from the first administration of BT1718, |
| | throughout the trial and for six months afterwards are considered eligible |
| 7. | Male patients with partners of child-bearing potential (unless they agree to take |
| | measures not to father children by using a barrier method of contraception [condom |
| | plus spermicide] or to sexual abstinence* effective from the first administration of |
| | BT1718, throughout the trial and for six months afterwards. Men with partners of |
| | child-bearing potential must also be willing to ensure that their partner uses an |
| | effective method of contraception for the same duration for example, hormonal |
| | contraception, intrauterine device, diaphragm with spermicidal gel or sexual |
| | abstinence). Men with pregnant or lactating partners must be advised to use barrier |
| | method contraception (for example, condom plus spermicidal gel) to prevent exposure |
| | of the foetus or neonate. |
| 8. | Surgery from which the patient has not yet recovered. |
| 9. | At high medical risk because of non-malignant systemic disease including active |
| | uncontrolled infection. |
| 10. | Known to be serologically positive for hepatitis B, hepatitis C or human |
| | immunodeficiency virus (HIV). |
| 11. | Concurrent congestive heart failure, prior history of class III/IV cardiac disease (New |
| | York Heart Association [NYHA]) Appendix 3, prior history of cardiac ischemia or |
| | prior history of clinically significant cardiac arrhythmia. |
| 12. | Previous known allergy to one of the constituents or excipients of BT1718. |
| 13. | Is a participant or plans to participate in another interventional clinical trial, whilst |
| | taking part in this Phase I/IIa study of BT1718. Participation in an observational trial |
| | or interventional clinical trial which does not involve administration of an IMP and |
| | which would not place an unacceptable burden on the patient in the opinion of the |
| | Investigator and Medical Advisor would be acceptable. |
| 14. | Any other condition which in the Investigator’s opinion would not make the patient a |
| | good candidate for the clinical trial. |

*Abstinence is only considered to be an acceptable method of contraception when this is in line with the preferred and usual lifestyle of the subject. Periodic abstinence (e.g., calendar, ovulation, symptothermal, post-ovulation methods) and withdrawal are not acceptable methods of contraception.

Patient Enrollment

Before enrolling the patient in the trial, the Investigator or designated representative should determine the eligibility of the patient during the trial screening period.

Eligible patients must be enrolled in the electronic data capture (EDC) system by site staff and then registered at the Centre for Drug Development (CDD) before they start treatment with BT1718. Eligible patients will be allocated a study number by the EDC system during the enrolment process. The CDD will send confirmation of the patient registration including the assigned dose level to the Investigator following enrolment of the patient. Study treatment may only be administered after confirmation has been received.

Treatment

Selection of the Phase I Starting Dose and Schedule

The HNSTD established in the 28 day-repeat dose GLP monkey study was 18 mg/m² twice weekly. Using allometric scaling and applying a standard safety factor of 6 (ICH S9), the human starting dose would be 3 mg/m² twice weekly. In a GLP-compliant study in the rat, the MTD was not reached with the highest administered dose 6 mg/m² twice weekly, with allometric scaling and a standard safety factor of 10 (ICH S9), the human starting dose would be 0.6 mg/m² twice weekly (which equates to a DM1 dose of approximately 0.12 mg/m² twice weekly). As the rat is the more sensitive species to BT1718, the starting dose will be based on the MTD of the rat.

Therefore, the proposed starting dose for the FIH Phase I trial is 0.6 mg/m² twice weekly.

Dosing Schedule/Treatment Schedule

Phase I, Dose Escalation Phase, Stage 1, Dosing Schedule/Treatment Schedule

BT1718 will be administered intravenously twice weekly for three out of four weeks (dosing on Days 1, 4, 8, 11, 15 and 18+/−1 day). Each cycle of treatment will consist of 28 days, and patients may continue until disease progression, depending on the availability of BT1718. The starting dose will be 0.6 mg/m².

Phase I, Dose Escalation Phase, Stage 2, Dosing Schedule/Treatment Schedule

In addition to evaluating a twice weekly dosing schedule in Stage 1 above, a once weekly dosing schedule will also be evaluated in Stage 2. Stage 2 will open at a dose where there is expectation of potential biological activity based on available PK, toxicity and/or PD data from Stage 1.

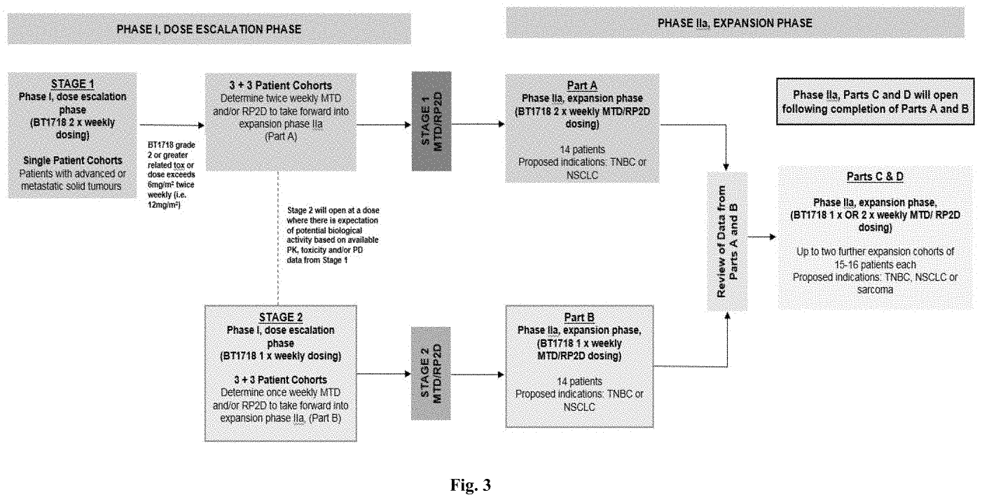

BT1718 will be administered intravenously once weekly for three out of four weeks (dosing on Days 1, 8 and 15+/−1 day). Each cycle of treatment will consist of 28 days and patients may continue until disease progression, depending on the availability of BT1718. The starting dose of the once weekly regime will be up to 100% of the overall weekly dose from the last completed cohort deemed safe from the twice weekly schedule (Phase I, Stage 1). Stage 2 will include a minimum of three evaluable patients following a 3+3 design and may be evaluated alongside recruitment to the Phase IIa, Part A twice weekly dose expansion phase (see FIG. 3).

Alternative dosing schedules may be considered by the Sponsor based on emerging data during the study, for example if the toxicology profile is benign with the twice weekly dosing regimen, continuous bi-weekly dosing may be evaluated. Emerging data obtained during Phase I may be used in the decision to proceed with the Phase IIa stages. In addition, depending on the patient populations selected, an increased dosing frequency may also be considered. If any changes are made to the dosing schedule a substantial amendment will be submitted to the MHRA, REC and HRA for approval.

Phase IIa, Expansion Phase (Part A)

BT1718 will be administered intravenously twice weekly at the RP2D and schedule defined by Phase I, Stage 1. Since Phase IIa, Part A, may begin when the RP2D for twice weekly dosing is available, this expansion may be evaluated in parallel with recruitment to an ongoing once weekly dose escalation in Phase I, Stage 2 (see above and FIG. 4).

Phase IIa, Expansion Phase (Part B)

BT1718 will be administered intravenously once weekly at the RP2D and schedule as defined by Phase I, Stage 2. Phase IIa, expansion phase Part B may begin when the RP2D for once weekly dosing is available therefore this expansion may be evaluated in parallel with the ongoing twice weekly dose escalation in Phase I, Stage 1 (if recruitment to that stage is ongoing) or the Phase IIa, expansion phase (Part A).

Phase IIa, Expansion Phase (Parts C & D)

Following completion of Parts A and B, results will be reviewed. Following review of the data generated to date in Phase I, Stages 1 and 2, and Phase IIa, Parts A and B, a decision will be made about which dosing scheme to employ (once or twice weekly), the starting dose and in which tumor indications. Following this decision, up to two additional cohorts of approximately 15-16 patients each will be enrolled in Parts C and D with patient populations chosen based on pre-clinical and clinically emerging data. These tumor types are currently proposed to include NSCLC, TNBC or Sarcoma. The selection biomarker or threshold may also be reviewed prior to Parts C and D.

Dose Escalation Scheme

Phase I, Dose Escalation Phase, Stage 1—(Twice Weekly Dosing)

In Phase I, dose escalation phase, Stage 1, dose increases will initially be up to a maximum of 100% in the single patient cohorts, driven by reported safety data and any available PK data, until the first CTCAE Grade 2 toxicity considered by investigators to be at least probably related to BT1718 is observed, or until the dose exceeds 6 mg/m² twice weekly (i.e. 12 mg/m² over the week). Subsequent cohorts will revert to a standard 3+3 format with dose escalation steps up to 100% driven by reported safety and available PK data. If a single DLT is seen among the initial three patients, the cohort will be expanded up to a total of six evaluable patients. The dose will be considered tolerable if less than two out of six evaluable patients experience a DLT.

In the single patient cohorts, the next patient can receive their first dose of BT1718 once the preceding patient has completed their DLT period (the first 28 days) and the Sponsor and study team has deemed it safe to proceed to the next cohort. In the 3+3 patient cohorts the first patient will be observed for toxicity for 7 days from Day 1 before subsequent patients receive their first dose of BT1718.

Patients who receive less than 75% of their planned doses during the first cycle (28 days DLT period) for reasons other than toxicity will not be evaluable for assessment of DLT for dose review decisions and may be replaced in the cohort. Reported safety information for these patients may however be considered to guide the percentage change in dose levels. In order to make the decision to escalate the BT1718 dose, the required number of evaluable patients must have completed one cycle (approximately 28 days).

Phase I, Dose Escalation Phase, Stage 2—(Once Weekly Dosing)

In Phase I, dose escalation phase (Stage 2) dose increases may be up to 100% of the previous dose level, and will be driven by reported safety and available PK data. If a single DLT is seen among the initial three patients, the cohort will be expanded up to a total of six evaluable patients. The dose will be considered tolerable if less than two out of six evaluable patients experience a DLT.

In the 3+3 patient cohorts the first patient will be observed for toxicity for 7 days from Day 1 before subsequent patients receive their first dose of BT1718.

Patients who receive less than 75% of their planned doses during the first cycle (28 days DLT period) for reasons other than toxicity will not be evaluable for assessment of DLT for dose review decisions and may be replaced in the cohort. Reported safety information for these patients may however be considered to guide the percentage change in dose levels. In order to make the decision to escalate the BT1718 dose, the required number of evaluable patients must have completed one cycle (approximately 28 days).

Intra-Patients Dose Escalations

No intra-patient dose escalation will be allowed.

Expansion of Dose Levels

If one instance of DLT as defined above is observed in a cohort of three patients, up to six patients will be treated at that dose level. If one out of six patients experiences a DLT, dose escalation will continue. If two or more out of up to six patients experience a DLT, dose escalation will stop and this dose will be defined as non-tolerated. A maximum of six evaluable patients will be treated at a dose below the non-tolerated level to define the MTD.

The MAD will be defined as the highest dose received. This will usually represent the non-tolerated dose above the MTD, but may instead represent a maximum administrable dose if the feasible volume of infusion limits the dose before toxicity does.

If a new type of DLT or high number of DLTs occurs during the dose expansion phases at the RP2D, the dose level for new patients in the expansion cohorts may be reduced based on the ongoing safety reporting. This will be continually monitored but also formally assessed after the first six patients have received two cycles of treatment.

Dose Modifications

Dose Delays and Reductions

Patients who experience a DLT (defined in Cycle 1 only) that resolves to Grade <1 or recovers to baseline within 15 days of the start of the DLT may recommence treatment, with the agreement of the PI, Sponsor and patient. The dose should be reduced to the previous dose level. If the AE has not resolved or recovered to baseline within 15 days, the patient will be taken off-study. If the patient experiences a DLT at this reduced dose, either the same or different toxicity, there will be no further dose reductions and the patient will be withdrawn from the study.

Hematological Toxicities

Patients who experience a ≥Grade 3 hematological toxicity will have the subsequent doses omitted during that cycle until the toxicity resolves to <Grade 3. On resolution to <Grade 3, treatment can recommence at the same dose level during that cycle.

If a Grade 2 toxicity related to BT1718 (probably or highly probably) is still present when a patient is due to start the next cycle, that cycle should be delayed up to 14 days until the toxicity resolves to ≤Grade 1.

The first time a dose is omitted or delayed, it may be given at the same dose the next time. However, if there is a subsequent need to omit or delay again, the dose should be reduced to the previous dose level (unless, in exceptional circumstances, the PI, Sponsor and patient agree that further dose omissions/delays are appropriate and provide effective control of toxicity).

If toxicity does not recover to <Grade 2 within 15 days, the patient will be taken off-study.

Only one dose reduction will be allowed per patient unless, in exceptional circumstances, the PI, Sponsor and patient agree that further treatment is appropriate and that a dose reduction is expected to provide effective control of toxicity.

Non-Hematological Toxicities:

Grade 2

Patients who experience a clinically-significant Grade 2 non-hematological toxicity related to BT1718 (probably or highly probably), that does not respond (<7 days) to supportive clinical management, will omit subsequent doses during that cycle until the toxicity resolves to ≤Grade 1.

If a clinically-significant Grade 2 BT1718-related toxicity is still present when a patient is due to start the next cycle, that cycle should be delayed up to 14 days until the toxicity resolves to ≤Grade 1.

The first time a dose is omitted or delayed, it may be given at the same dose when recommencing. However, if there is a subsequent need to omit or delay again, the dose should be reduced to the previous dose level (unless, in exceptional circumstance, the PI, Sponsor and patient agree that further dose omissions/delays are appropriate and provide effective control of toxicity).

If toxicity does not recover to ≤Grade 1 within 15 days, the patient will be taken off-study.

Only one dose reduction will be allowed per patient, unless, in exceptional circumstances, the PI, Sponsor and patient agree that further treatment is appropriate and that a dose reduction is expected to provide effective control of toxicity.

Grade 3

Patients who experience a Grade 3 non-hematological toxicity that does not rapidly respond (<3 days) to supportive clinical management, will omit subsequent doses during that cycle until the toxicity resolves to ≤Grade 1.

If clinically-significant Grade 2 BT1718-related toxicity is still present when a patient is due to start the next cycle, that cycle should be delayed up to 14 days until the toxicity resolves to ≤Grade 1.

When patients recommence treatment after a Grade 3 non-hematological toxicity, the dose should be reduced to the previous dose level. If toxicity does not recover to ≤Grade 1 within 15 days, the patient will be taken off-study.

Only one dose reduction will be allowed per patient, unless, in exceptional circumstances, the PI, Sponsor and patient agree that further treatment is appropriate and that a dose reduction is expected to provide effective control of toxicity.

Grade 4

Patients who experience a Grade 4 non-hematological toxicity, or liver enzyme changes consistent with Hy's Law (bilirubin >2×ULN and ALT/AST>3×ULN, with no explanation other than drug), will cease further treatment and be taken off-study once accuracy of testing is confirmed.

Duration of Treatment

Treatment should continue unless (a) the patient asks to be withdrawn, (b) there is evidence of disease progression, (c) the patient is experiencing unacceptable toxicity or (d) the Investigator feels the patient should be withdrawn for any other reason.

If the Sponsor and CI agree that a patient is benefiting from treatment with BT1718 (i.e. has stable or responding disease as measured by RECIST 1.1) and is not experiencing any clinically significant Grade 2 or greater BT1718-related AEs, the patient may continue with treatment until disease progression (depending on the availability of BT1718), after which the patient will be withdrawn from the trial. Follow up information should be collected as described in the protocol (see below).

The Sponsor will review a full toxicity and efficacy profile including radiological data to confirm the reported objective response for that patient when considering whether the patient should continue to receive treatment. If the Sponsor decides not to allow the patient to continue treatment based on the information provided or on other information received, or for any other reason, then the Sponsor's decision is final.

Replacement of Patients

Phase I, Dose Escalation Phase (Stage 1 and Stage 2)

Patients will be replaced by another patient treated at the same dose level during the dose escalation phase if they receive less than 75% of planned dose exposure of BT1718 during the first cycle (28 day DLT period) for reasons other than BT1718-related (probably or highly probably) toxicity. For single patient cohorts, patients may be replaced if they do not receive all their planned doses.

Phase II, Expansion Phase

Patients will be replaced in the expansion cohorts (Parts A, B, C and D) if they receive less than 75% of the planned dose exposure of BT1718 during the first cycle (28 DLT period) for reasons other than drug-related (probably or highly probably) toxicity, unless there is evidence of PD.

Replacement of patients will be confirmed by the Sponsor. There may be circumstances based on the emerging data from the trial or BT1718 availability which result in a patient not being replaced. This will be documented by the Sponsor.

Concomitant Medication and Treatment

Concomitant medication may be given as medically indicated. This includes symptomatic clinical management of BT1718 related or unrelated AEs. Details (including name and start and stop dates of the concomitant medication given) must be recorded in the patient's medical records and details entered into the electronic case report form (eCRF).

Palliative radiotherapy may be given concomitantly for the control of bone pain or other symptoms. These irradiated lesions will not be evaluable for response.

The patient must not receive other anti-cancer therapy or investigational drugs while on the trial.

As described above, dosing with BT1718 may also be delayed up to 15 days if required to manage toxicity, without a patient needing to be withdrawn from study.

Sun Exposure Precautions

BT1718 absorbs in the UV (ultraviolet) spectrum (290 to 700 nm), specifically between 290-300 nm, with a molar extinction coefficient >1000 L mol⁻¹ cm⁻¹. As such, although DM-1 containing drugs have not shown evidence of phototoxicity in patients, it must be considered a possibility and precautions around UV exposure are required whilst on treatment and for one week afterwards. Patients should avoid excessive sun exposure and when outdoors during the daytime, patients should wear protective clothing, including a hat and sunglasses where appropriate, and apply broad spectrum sunscreen with a high sun protection factor (SPF30 or above) to any potentially exposed skin. Sun beds are not to be used.

Pharmaceutical Information

Pharmaceutical Data

Formulation of BT1718

BT1718 drug product will be supplied for clinical use as a white to off white lyophilised powder for reconstitution in a 20 mL Type I clear glass vial with a chlorobutyl stopper and aluminium seal. The actual drug content of the vial will be 21.2 mg of BT1718.

Storage Conditions

All supplies must be stored in a secure, limited access storage area in the hospital pharmacy. BT1718 must be stored in its original packaging at −20° C.±5° C., protected from light.

Method of Preparation of the IMP

Good aseptic practice must be employed when preparing solutions of BT1718 for infusion.

BT1718 will be reconstituted with 5.0 mL of sterile water for injections (to return a target volume of 5.3 mL solution) to provide BT1718 at a target concentration of 4.0 mg/mL for further dilution with 5% dextrose prior to IV administration (by infusion). Vial content ensures availability of 20 mg BT1718 per vial based on the withdrawable volume of 5.0 mL following reconstitution.

Stability of the Diluted BT1718

The prepared solution for infusion may be stored at 2-8° C. for 20 hours, followed by 4 hours at room temperature (21° C.±4° C.) before administration. Infusion must be completed within 2 hours. From a microbiological point of view administration should take place as soon as possible after preparation of the diluted drug.

BT1718 Administration

Before administration, the exact dosage must always be double-checked by a second suitably qualified person.

Monitoring During Infusion

Vital signs (temperature, pulse rate, BP) should be monitored before and after the infusion and should be repeated if any concerns during treatment or observation. Patients with diabetes mellitus should have a glucose finger-prick test before and after each BT1718 infusion for at least the first two cycles and thereafter if clinically indicated.

Patients should not receive primary prophylaxis with any premedication prior to their first infusion of BT1718 in order to fully assess any BT1718 adverse effects. Should a patient exhibit any adverse effects to BT1718, then a premedication may be administered as secondary prophylaxis prior to any subsequent infusions (e.g. dexamethasone, chlorphenamine or an anti-emetic). However, should emerging safety data suggest that a premedication is necessary as primary prophylaxis for all patients, this will be given prior to Cycle 1 Day 1 and for all infusions going forward. The Sponsor will ensure this requirement is communicated to each investigator.

Granulocyte-macrophage colony-stimulating factor (GM-CSF) or granulocyte-colony stimulating factor (GCSF) should not be used a primary or secondary prophylaxis during the study, nor solely to accelerate marrow recovery to increase dose density. However, the therapeutic use of GM-CSF or GCSF is permitted if there is an acute clinical requirement for bone marrow support (e.g. therapeutically in the case of febrile neutropenia). If at all possible, its use should be avoided within the patient's first cycle of BT1718 as establishing the duration of any leukopenia, neutropenia, erythropenia or thrombocytopenia forms part of the DLT assessment, however the patient's safety and wellbeing remain the primary concern in clinical decisions about bone marrow support.

Vein Extravasation/Accidental Spillages

DM1 and other chemotherapeutics can cause extravasation and/or are vesicants, irritants, inflammitants or exfoliants [56, 57] and as a precaution BT1718 will be treated as a vesicant. Careful attention should be paid to cannula siting, patency and any indication of extravasation during or after infusion. Standard local policies for management of vesicant extravasation should be followed, typically starting with stopping the infusion, aspirating if possible, topical hydrocortisone and ongoing review. The role of specific treatment such as heat or cold packs, DMSO or hyaluronidase is unknown. Gloves and a disposable apron should be worn at all times during preparation, checking, administration, disposable or management of spillage of BT1718.

Investigations Schedule

In cases where a patient has investigations at a different hospital, for example weekly blood samples, scans and other investigations as appropriate, then it is the Investigator's responsibility to ensure he/she receives and reviews the reported results. These results must be available for source data verification (SDV). Laboratory reference ranges, including effective dates, and evidence of laboratory accreditation must be obtained from all laboratories used. For scan results, the original images and reports must be available for comparison to any scan performed at the investigator site and be in a format that is suitable for comparison. For all other investigations, apart from the results, any supporting data must be made available for SDV or review.

The Investigator or delegate must inform the Sponsor of any changes to the laboratory normal ranges or to any laboratory accreditation and provide any new documentation.

Pre-Treatment Evaluations

Details of all evaluations/investigations for enrolled patients, including relevant dates, required by the protocol must be recorded in the medical records.

Obtaining Written Informed Consent

Written informed consent must be obtained from the patient before any protocol-specific procedures are carried out.

Consent for analysis of initial archived or fresh screening tumor sample for MT1-MMP must be obtained prior to analysis of the sample for the trial and should be obtained pre-screening (prescreening consent) or at the time of full trial consent (main consent form).

The patient must be given adequate time to think about their commitment to the study. If more than 28 days has passed since informed consent was obtained before the start of BT1718 dosing then the Investigator should consider whether repeat consent should be obtained from a patient. Should a newer approved version of the informed consent document (ICD) be available, then re-consent must be obtained before any protocol specific investigations are performed.

Only the PI and those Sub-Investigator(s) with delegated responsibility by the PI, and who have signed the Delegation Log, are permitted to obtain informed consent from patients and sign the consent form. All signatures must be obtained before the occurrence of any medical intervention required by the protocol. The patient should sign and date the consent form in the presence of the Investigator, followed by the Investigator signature. The date of the signatures of both the patient and the PI/Sub-Investigator obtaining informed consent should be the same.

The PI or the Sub-Investigator must inform the patient about the background to, and present knowledge of the normal management of their disease and BT1718 and must also ensure that the patient is aware of the following points:

That BT1718 is new and that the exact degree of activity is at present unknown, but that treating him/her will contribute to further knowledge;

The known toxicity of BT1718 and the possibility of experiencing side-effects;

The potential dangers of becoming pregnant (or the patient's partner becoming pregnant) and he/she has been given information about appropriate medically approved contraception;

That he/she may refuse treatment either before or at any time during the trial and that refusal to participate will involve no penalty or loss of benefits to which they are otherwise entitled;

Whom to contact for answers to pertinent questions about the research and their rights, and also who to contact in the event of a research-related injury.

Pre-Screening (Phase IIa, Expansion Phase Only)

All patients in Phase IIa, expansion phase, should give separate written consent before sending archival tissue, or before obtaining and sending a fresh screening tumor biopsy sample, for MT1-MMP IHC profiling.

The following should be performed/obtained within six months before the patient receives the first dose:

Archival Sample Retrieval—Retrieval of Archival Tumor Sample for MT1-MMP IHC Profiling

Fresh tumor biopsy sample—if archival tumor sample is not available, obtain a fresh tumor biopsy sample for MT1-MMP IHC profiling.

For those patients where an archival tumor biopsy sample is not available or MT1-MMP IHC profiling is inconclusive, a fresh screening tumor biopsy will be required to determine eligibility. The fresh screening tumor biopsy sample must be performed/obtained no more than eight weeks before the patient is expected to be enrolled.

Evaluations within 28 Days Prior to First Administration of BT1718 (Day −28 to Pre-Dose on Cycle 1 Day 1)

The following must be performed/obtained within 28 days before the patient receives their first dose of BT1718. Existing results such as radiological measurements may be used even where these investigations were performed prior to the patient's provision of information consent for the study if they were performed within the required time window.

Demographic Details

Medical history including diagnosis (histological or cytological), prior treatment, concomitant conditions/diseases, baseline signs and symptoms and concomitant treatment)

Radiological disease assessments: radiological measurements (computerized tomography [CT] or magnetic resonance imaging [MRI] scan of the chest, abdomen, pelvis and any other relevant sites)—must be performed within four weeks before the patient receives the first dose of BT1718 and reported to RECIST Version 1.1

Retrieval of archival tumor sample for retrospective assessment of MT1-MMP expression by IHC, as well as other molecular pathology techniques in the dose escalation phase.

Pre-treatment tumor biopsy optional for patients in the dose escalation phase (Phase I, Stage 1 and 2), mandatory for a minimum of six patients in the expansion phase (Phase IIa, Parts A and B).

Note: The baseline pre-treatment tumor biopsy (performed within 28 days prior to the first dose) will not be required for those patients who have provided a fresh screening tumor sample (to confirm eligibility by MT1-MMP IHC profiling) within eight weeks prior to the first dose.

Optional fresh non-tumor biopsy sample: Patients who have a pre-treatment tumor biopsy may also have an optional fresh non-tumor biopsy. The non-tumor biopsy can be taken around the same time as the tumor biopsy. This applies to patients in the dose escalation phase (Phase I, Stage 1 and 2) and in the expansion phase (Phase IIa, Parts A and B).

Clinical Disease Assessments (if Applicable)

Note that all AEs, including SAEs, must be monitored and recorded in the eCRF from the time the patient consents to any protocol-specific procedure.

Evaluations within 8 Days of Study Inclusion

The following must be performed within 8 days before study inclusion:

Complete Physical Examination

Height, weight, BSA, WHO performance status, temperature, pulse rate and BP (BP to be taken seated or lying after 5 minutes rest)

12 Lead ECG;

Female patients able to have children must have a negative result on a human chorionic gonadotropin (HCG) pregnancy test (serum or urine test is acceptable) prior to first BT1718 administration and 30 days after final administration only (Note more frequent testing is unlikely to be appropriate unless specific concern, given patients' advanced disease, their limited prognosis, the unlikeliness of pregnancy, and sensitives around reminders of how cancer has impacted on their future/fertility/family-life).

Laboratory Tests (Blood/Urine Samples):

Hematology—Hb, WBC with two-point differential count (neutrophils, lymphocytes) and platelets

Biochemistry—sodium, potassium, adjusted calcium, magnesium, phosphate, urea, creatinine, albumin, bilirubin, ALP, ALT and/or AST, fasting glucose

Urinalysis—including pH, glucose, protein and blood

Renal function—serum creatinine, calculated creatinine clearance (Wright or Cockroft & Gault) or Isotope clearance measurement (GFR scan). Isotope clearance result to be used if calculated C&G/Wright method results in a GFR=50 mL/min.

Coagulation tests—including INR/PT and APTT. Should be repeated before any biopsy procedure.

Enroll the patient on the study once confirmed as eligible.

Research blood samples: for pharmacodynamics and genetic analysis.

Evaluations During the Trial

Day 1 of Each Cycle

The following must be performed on Day 1 of each cycle before BT1718 administration:

Weight and BSA—must be repeated on Day 1 of each cycle to calculate the dose required for BT1718 administration. The dose of BT1718 will only need to be recalculated should there be a 10% or greater change in weight since baseline, since last weight adjustment or where doses are reduced because of toxicity

12 lead ECG—should be performed before BT1718 administration, and within 1½ hours (+15 mins) after BT1718 administration during Cycle 1 Day 1. On Cycle 2 Day 1 an ECG should be performed either before or within 1½ hours (+15 mins) after BT1718 administration. From Cycle 2 onwards ECGs should be repeated before or within 1½ hours (+15 mins) after BT1718 administration on Day 1 every two cycles (e.g. Cycle 4 Day 1, Cycle 6 Day 1).

Hematology: detailed in paragraph [00342].

Biochemistry: detailed in paragraph [00343]. A fasting glucose result is not required except for baseline so, there is no need for the patient to fast before attending the hospital;

Urinalysis—detailed in paragraph [00344]. This assessment should be repeated on Day 22 (+/−1 day) of Cycle 1 only (i.e. when no BT1718 administration takes place).

Research urine sample collection; for PK analysis.

Research blood samples: for PK, pharmacodynamics and genetic analysis.

Prior to Each Planned BT1718 Administration

Symptom-directed physical examination: if clinically indicated, a symptom-directed physical examination is to be performed before BT1718 administration.

WHO Performance Status

Vital signs: Temperature, pulse rate and BP (BP to be taken seated or lying after 5 minutes rest) performed before BT1718 administration and 1 hour (+/−15 mins) post infusion. Should also be done during infusion if clinically indicated. These assessments should be repeated on Day 22 (+/−1 day) of Cycle 1 only (i.e. when no BT1718 administration takes place).

Patients with diabetes mellitus should have a glucose finger-prick test (non-fasting) within 1 hour before and 1 hour (+15 mins) after each BT1718 infusion for at least the first two cycles and thereafter if clinically indicated.

Adverse events and concomitant medications: At each visit, before each BT1718 administration, an assessment of any AE experienced since the previous visit must be made by the Investigator, Research Nurse or suitably qualified member of the Investigator's team. The start and stop dates of the AE together with the relationship of the event to the BT1718 administration must be recorded in the medical records. All AEs must be graded according to NCI CTCAE Version 4.02. Any concomitant treatment must be recorded in the medical records.

Laboratory Tests:

Hematology and Biochemistry:

Hematology: detailed in paragraph [00342].

Core biochemistry: sodium, potassium, urea, creatinine, albumin, bilirubin, ALP, ALT and/or AST.

Phase I, dose escalation phase, Stage 1, and Phase IIa, Part A (twice weekly dosing)

During Cycle 1 laboratory tests (as defined above) must be performed and checked prior to BT1718 administration on Days 1, 4, 8, 11, 15 and 18. Additional laboratory tests must also be performed on Day 22 (i.e. when no BT1718 administration takes place). Laboratory tests may be performed up to 24 hours prior BT1718 administration but results must be available and reviewed by the Investigator before BT1718 is given.

During Cycle 2 onwards laboratory tests must be performed and checked prior to BT1718 administration on Days 1, 8 and 15 and thereafter if clinically indicated. After six cycles, the frequency of haematology and biochemistry assessments may decrease at the discretion of the PI and Sponsor but, at a minimum, must be performed on Day 1 of each cycle. Laboratory tests may be performed up to 24 hours prior BT1718 administration but results must be available and reviewed by the Investigator before BT1718 is given.

Phase I, Dose Escalation Phase, Stage 2, and Phase IIa, Part B (Once Weekly Dosing)

Laboratory tests must be performed and checked prior to BT1718 administration on Days 1, 8 and 15. Additional laboratory tests must also be performed on Day 22 (i.e. when no BT1718 administration takes place). Laboratory tests may be performed up to 24 hours prior BT1718 administration but results must be available and reviewed by the Investigator before BT1718 is given.

After six cycles, the frequency of hematology and biochemistry assessments may decrease at the discretion of the PI and Sponsor but, at a minimum, must be performed on Day 1 of each cycle. Laboratory tests may be performed up to 24 hours prior BT1718 administration but results must be available and reviewed by the Investigator before BT1718 is given.

Radiological disease assessment: This must be repeated at the end of every two cycles (+/−7 days). Assessments may continue until disease progression for up two years and can be performed more frequently than every two cycles, if clinical concern or suspicion of disease progression. Radiological measurements (CT or MRI scan of the chest, abdomen, pelvis and any other relevant sites)—reported to Response Evaluation Criteria in Solid Tumors (RECIST) Version 1.1

Clinical disease assessment (if applicable): This must be repeated at the end of every two cycles (+/−7 days) until disease progression for up two years, or if clinical concern or suspicion of disease progression.

Other Assessments During BT1718 Treatment:

A post treatment tumor biopsy is optional for patients in the dose escalation phase (Phase I, Stage 1 and 2) and mandatory for a minimum of six patients in the expansion phase (Phase IIa, Parts A and B). A tumor biopsy for pharmacodynamics assessment will be taken on Cycle 1 Day 8 or Day 15 (4+/−2 hours). If unable to perform the biopsy procedure during Cycle 1, this may be performed during Cycle 2 Day 8 or Day 15 (4+/−2 hours).

A post treatment non-tumor biopsy is optional for patients in the dose escalation phase (Phase I, Stage 1 and 2) and expansion phase (Phase IIa, Parts A and B). A non-tumor biopsy for pharmacodynamics assessment will be taken from the same patient having a post treatment tumor biopsy. The non-tumor biopsy can be taken around the same time as the tumor biopsy in Cycle 1 Day 8 or Day 15 (4+/−2 hours post treatment). If unable to perform the biopsy procedure during Cycle 1, this may be performed during Cycle 2 Day 8 or Day 15 (4+/−2 hours post treatment).

Research blood samples: for pharmacodynamics and genetic analysis.

NB: Evaluations for Phase IIa, expansion phase Parts C and D will be defined following completion of expansion phase Parts A and B.

Evaluations at Off-Study Visit

Off study is defined as the date the decision is taken to withdraw the patient from the trial. Evaluations at the ‘off-study’ visit must be performed 28 days (+/−7 days) after the last dose of BT1718. The following investigations should be performed wherever possible:

Symptom-directed physical examination: if clinically indicated;

WHO performance status, temperature, pulse rate and BP (BP to be taken seated or lying after 5 minutes rest);

Hematology tests: detailed in paragraph [00342];

Biochemistry tests: detailed in paragraph [00343]; A fasting glucose result is not required, there is no need for the patient to fast before attending the hospital;

Urinalysis; detailed in paragraph [00344];

Female patients able to have children must have a negative result on a HCG pregnancy test (serum or urine test is acceptable);

12 Lead ECG;

Radiological assessment of tumor disease, unless the patient has been shown to have PD on a previous study scan or an assessment has been performed within the previous 28 days;

Clinical assessment of disease if applicable;

Assessment of AEs (also see above);

Review of concomitant medications; and

Research blood samples: for pharmacodynamics and genetic analysis.

Follow-Up

Safety Follow-Up

For eligible patients, SAE and AE collection and monitoring will continue until 28 days after the last administration of BT1718 or until the patient starts another anti-cancer therapy. Any drug-related AEs still ongoing after this period will be followed up monthly until resolution to baseline or stabilization, unless the patient starts another anti-cancer treatment.

Should an Investigator become aware of any BT1718-related AEs or SAEs after this period, these must also be reported to the Sponsor within the expedited timelines.

Efficacy and Survival Follow-Up

All patients should be followed up for first progression and for survival until the end of trial. For those no longer on treatment, the site trial team should check the status of the patient at least three monthly (through NHS/HSC electronic data records or by phone calls only if appropriate) to determine when the patient starts another systemic anti-cancer therapy or when PD occurs (if not already occurred) and if the patient remains alive. Patients who are no longer on treatment no further trial visits are required, but site team should check the status of the patient.

If the patient is lost to follow-up or have not progressed or died at the time of the final database lock for the Clinical Study Report (CSR), then the information will be censored as not known to have progressed/died at that time.

Schedule of Events

TABLE 10
Phase I, stage 1 and Phase IIa, Part A (twice weekly dosing).

| | Pre- | Pre-treatment evaluations | | | | | | | | | | | | |
|---|---|---|---|---|---|---|---|---|---|---|---|---|---|---|
| | screening | (screening period) | | | | | | | | | Cycle 2 | Off study | | Survival |
| | (Expansion | Within | Within | | | | | | | | onwards | Off study: | Follow-up | Follow-up |
| | Phase only) | 28 days | 8 days | Cycle 1 | | | | | | | Days 1, 4, | (28 ± 7 days | At | Until |
| Observation/ | 6 months | (Day-28 to | before | Day | Day | Day | Day | Day | Day | Day | 8, 11, 15 | after last dose | least | end of |
| Investigation | to Day-7 | Pre-dose Day 1) | study inclusion | 1 | 4 | 8 | 11 | 15 | 18 | 22 | and 18 | of IMP) | monthly | trial |
| Written informed | X | X | | | | | | | | | | | | |
| consent | | | | | | | | | | | | | | |
| Archival or fresh | X | | | | | | | | | | | | | |
| tumor biopsy for | | | | | | | | | | | | | | |
| MT1-MMP ICH | | | | | | | | | | | | | | |
| profiling (a) | | | | | | | | | | | | | | |
| Demographics | | X | | | | | | | | | | | | |
| Medical history | | X | | | | | | | | | | | | |
| Adverse event | | From date of informed consent | | Continually review | | | | | | | X | Until | | |
| evaluation | | | | | | | | | | | | resolution | | |
| | | | | | | | | | | | | (m) | | |
| Concomitant | | From date of informed consent | | Continually review | | | | | | | X | | | |
| treatments | | | | | | | | | | | | | | |
| Radiological | | X | | At the end of every two cycles (p) | | | | | | | X (l) | | | |
| disease assessment | | | | | | | | | | | | | | |
| Clinical disease | | X | | At the end of every two cycles | | | | | | | X | | | |
| assessment (if | | | | | | | | | | | | | | |
| applicable) | | | | | | | | | | | | | | |
| Pregnancy test (b) | | | X | | | | | | | | X | | | |
| Physical | | | X | Synptom-directed, repeat as | | | | | | | Symptom- | | | |
| examination (c) | | | | clinically indicated | | | | | | | directed | | | |
| | | | | | | | | | | | (if clinically | | | |
| | | | | | | | | | | | indicated) | | | |
| Height | | | X | | | | | | | | | | | |
| Weight | | | X | X | | | | | | | X (Day 1 | | | |
| | | | | | | | | | | | only) | | | |
| Body surface | | | X | X | | | | | | | X (Day 1 | | | |
| area (BSA) | | | | | | | | | | | only) | | | |
| Bloods for | | | X | X | X | X | X | X | X | X | X (Day 1, 8 | X | | |
| hematology and | | | | | | | | | | | and 15 only) | | | |
| biochemistry (e) (f) | | | | | | | | | | | | | | |
| Coagulation tests | | | X | | | | | | | | | | | |
| (g) | | | | | | | | | | | | | | |
| Renal function | | | X | | | | | | | | | | | |
| Urinalysis | | | X | X | | | | | | X | X (Day 1 | X | | |
| | | | | | | | | | | | only) | | | |
| Glucose finger- | | | | X | X | X | X | X | X | | X | | | |
| prick test before | | | | | | | | | | | | | | |
| and after BT1718 | | | | | | | | | | | | | | |
| administration | | | | | | | | | | | | | | |
| (diabetic patients | | | | | | | | | | | | | | |
| only) (h) | | | | | | | | | | | | | | |
| Electrocardiogram | | | X | X | | | | | | | X (Day 1 | X | | |
| (ECG) (i) | | | | | | | | | | | only) | | | |
| BT1718 | | | | X | X | X | X | X | X | | X | | | |
| administration | | | | | | | | | | | | | | |
| (+/−1 day) | | | | | | | | | | | | | | |
| Temperature, | | | X | X | X | X | X | X | X | X | X | X | | |
| blood pressure | | | | | | | | | | | | | | |
| (seated or lying | | | | | | | | | | | | | | |
| after 15 mins rest) | | | | | | | | | | | | | | |
| and pulse (q) | | | | | | | | | | | | | | |
| WHO performance | | | X | X | X | X | X | X | X | X | X | X | | |
| status | | | | | | | | | | | | | | |
| Tumor biopsies (j) | X | Or X | | | | X | | Or X | | | Or X (j) | | | |
| | | | | | | (j) | | (j) | | | | | | |
| Optional non- | | X | | | | X | | Or X | | | Or X (p) | | | |
| tumor biopsy (o) | | | | | | (o) | | (o) | | | | | | |
| Blood for PK (o) | | | | Refer to Lab manual for exact timings | | | | | | | | | | |
| Blood for | | | | Refer to Lab manual for exact timings | | | | | | | | | | |
| Pharmacodynamics | | | | | | | | | | | | | | |
| (k) | | | | | | | | | | | | | | |
| Blood for ADA | | | | Refer to Lab manual for exact timings | | | | | | | | | | |
| Urine sample for PK | | | | Refer to Lab manual for exact timings | | | | | | | | | | |
| Follow-up contact | | | | | | | | | | | | | | X |
| for survival/ | | | | | | | | | | | | | | |
| progression (n) | | | | | | | | | | | | | | |

(a) Archival sample if available in Phase I, the dose escalation phase (retrospective MT1-MMp status). Tumor sample, whether archival or fresh, mandatory in Phase IIa, the expansion phase to confirm patient eligibility. Archival tumor biopsy sample can be obtained within six months prior to the first dose. Fresh screening tumor biopsy must be performed/obtained within eight weeks prior to first dose.
(b) Pregnancy test: For female patients of child bearing potential.
(c) Complete physical examination to be performed Pre-treatment then all subsequent examinations can be symptom-directed and only performed as clinically indicated.
(d) Screening weight can be used to calculate BSA for Cycle 1.
(e) Clinical laboratory assessments should be performed within the previous seven days of Cycle 1 Day 1 and prior to each planned BT1718 administration on Days 1, 4, 8, 11, 15, 18 and 22 of Cycle 1. From Cycle 2 onwards, laboratory assessments must be performed and checked pror to BT1718 administration on Days 1, 8 and 15 and thereafter if clinically indicated. After six cycles, the frequency of haematology and biochemistry assessments may decrease at the direction of the PI and Sponsor but, at a minimum, must be performed on Day 1 of each cycle. Laboratory tests may be performed up to 24 hours prior to BT1718 administration but results must be available and reviewed by the Investigator before BT1718 is given. On Day 1 of each cycle a full biochemistry assessment should be performed and on subsequent dosing dayys core biochemistry assessment should be performed.
(f) In the event of a Grade 4 neutropenia or Grade 4 thrombocytopenia a full blood count must be performed at least on Day 7 (neutropenia) and Day 5 (thrombocytopenia) after the onset of the event to determine if a dose limiting toxicity has occured. Continue close monitoring until resolution to Grade 3 or less.
(g) To be repeated before biopsy procedure if done.
(h) To be done Cycle 1 and 2, within 1 hour before and 1 hour (+15 mins) after BT1718 administration, then clinically indicated thereafter.
(i) ECGs should be performed before BT1718 administration, annd within 1 ½ hours (+15 mins) after BT1718 administration during Cycle 1 Day 1. On Cycle 2 Day 1 an ECG should be performed before or within 1 1/2 hours (+15 mins) after BT1718 administration. From Cycle 2 onwards ECG should be repeated to Day 1 every two cycles.
(j) Pre-treatment tumor bopsy; optional for patients enrolled in the dose escalation phase (Phase I, Stage 1 and 2); mandatory for patients in the expansion phase (Phase IIa, Parts A and B) if no archival sample available for MT1-MMP screening; mandatory for a minimum of six patients in the expansion phase (Phase IIa, Parts A and B). Post-treatment tumor biopsy; optional for patients in the dose escalation phase (Phase I, Stage 1 and ); mandatory in a minimum of six patients in the expansion phase (Phase IIa, Parts A and B) in Cycle 1 on Day 8 or 15 performed 4 +/− 2 hours post treatment. If unable to perform the biopsy during Cycle 1, this may be performed 4 +/− 2 hours post treatment during Cycle 2 on Day 8 or 15.
(k) Pharmacokinetic (PK) and pharmacodynamics sample collection-refer to the laboratory Manual for specific timings.
(I) Unless performed within previous 28 days or PD seen on previous study scan.
(m) Monthly follow-up required ONLY for thos AEs and SAEs considered drug-related (highly probable, probable, or possible) and presetn at off-study visit. Monthly follow-up to continue until resolution, return to baseline, stabilisation or patient starts another anti-cancer treatment.
(n) All patients should be followed up for first progression and for survival until the end of trial. For those no longer on treatment, the site trial team should check the status of the patient at least three monthly (through NHS/HSC electronic data records or by phone calls only if appropriate) to determine when PD occurs (if not already occured) and if the patient remains alive.
(o) Optional non-tumor biopsy sample may also be taken from the same patient having the pre and post treatment tumor biopsies (i.e. during Cycle 1 Day 8 or 15 or Cycle 2 Day 8 or 15 performed 4 +/− 2 hours) in the dose escalation phase (Phase I, Stage 1 and 2) and expansion phase (Phase IIa, Parts A and B).
(p) The timing of CT/MRI schedule may change in the expansion phase (Phase IIa) depending on tumor type.
(q) Temperature, blood pressure (seated or lying after 5 mins rest) and pulse should be performed before and then repeated 1 hr (+/−15 mins) after BT1718 administration.

TABLE 11
Phase I, stage 1 and Phase IIa, Part A (twice weekly dosing).

| Pre- | Pre-treatment evaluations | | |
|---|---|---|---|
| screening | (screening period | Off study | Survival |

| | 6 months | Within | Within | | | | | Cycle 2 | Off study: | Follow-up | Follow-up |
|---|---|---|---|---|---|---|---|---|---|---|---|
| | to Day-7 | 28 days | 8 days | Cycle 1 | | | | onwards | (28 ± 7 days | At | Until |
| Observation/ | (Expansion | (Day-28 to | before | Day | Day | Day | Day | Days 1, | after last dose | least | end of |
| Investigation | Phase only) | Pre-dose Day 1) | study inclusion | 1 | 8 | 15 | 22 | 8 and 15 | of IMP) | monthly | trial |
| Written informed | X | X | | | | | | | | | |
| consent | | | | | | | | | | | |
| Archival or fresh | X | | | | | | | | | | |
| tumor biopsy for | | | | | | | | | | | |
| MT1-MMP ICH | | | | | | | | | | | |
| profiling (a) | | | | | | | | | | | |
| Demographics | | X | | | | | | | | | |
| Medical history | | X | | | | | | | | | |
| Adverse event | | From date of informed consent | | Continually review | | | | | X | Until | |
| evaluation | | | | | | | | | | resolution | |
| | | | | | | | | | | (m) | |
| Concomitant | | From date of informed consent | | Continually review | | | | | X | | |
| treatments | | | | | | | | | | | |
| Radiological | | X | | At the end of every two cycles (p) | | | | | X (l) | | |
| disease assessment | | | | | | | | | | | |
| Clinical disease | | X | | At the end of every two cycles | | | | | X | | |
| assessment (if | | | | | | | | | | | |
| applicable) | | | | | | | | | | | |
| Pregnancy test (b) | | | X | | | | | | X | | |
| Physical | | | X | Symptom-directed, repeat | | | | | Symptom- | | |
| examination (c) | | | | as clinically indicated | | | | | directed (if | | |
| | | | | | | | | | clinically | | |
| | | | | | | | | | indicated) | | |
| Height | | | X | | | | | | | | |
| Weight (d) | | | X | X | | | | X (Day | | | |
| | | | | | | | | 1 only) | | | |
| Body surface | | | X | X | | | | X (Day | | | |
| area (BSA) | | | | | | | | 1 only) | | | |
| Bloods for | | | X | X | X | X | X | X | X | | |
| hematology and | | | | | | | | | | | |
| biochemistry (e) (f) | | | | | | | | | | | |
| Coagulation tests | | | X | | | | | | | | |
| (g) | | | | | | | | | | | |
| Renal function | | | X | | | | | | | | |
| Urinalysis | | | X | X | | | X | X (Day | X | | |
| | | | | | | | | 1 only) | | | |
| Glucose finger- | | | | X | X | X | | X | | | |
| prick test before | | | | | | | | | | | |
| and after BT1718 | | | | | | | | | | | |
| administration | | | | | | | | | | | |
| (diabetic patients | | | | | | | | | | | |
| only) (h) | | | | | | | | | | | |
| Electrocardiogram | | | X | X | | | | X (Day | X | | |
| (ECG) (i) | | | | | | | | 1 only) | | | |
| BT1718 | | | | X | X | X | | | | | |
| administration | | | | | | | | | | | |
| (+/−1 day) | | | | | | | | | | | |
| Temperature, | | | X | X | X | X | X | X | X | | |
| blood pressure | | | | | | | | | | | |
| (seated or lying | | | | | | | | | | | |
| after 15 mins rest) | | | | | | | | | | | |
| and pulse (q) | | | | | | | | | | | |
| WHO performance | | | X | X | X | X | X | X | X | | |
| status | | | | | | | | | | | |
| Tumor biopsies (j) | X | Or X | | X (j) | | Or X | | Or X | | | |
| | | | | | | (j) | | (j) | | | |
| Optional non- | | X | | X (p) | | X (p) | | X (p) | | | |
| tumor biopsy (o) | | | | | | | | | | | |
| Blood for PK (k) | | | | Refer to Lab manual for exact timings | | | | | | | |
| Blood for | | | | Refer to Lab manual for exact timings | | | | | | | |
| Pharmacodynamics | | | | | | | | | | | |
| (k) | | | | | | | | | | | |
| Blood for ADA | | | | Refer to Lab manual for exact timings | | | | | | | |
| Urine sample for PK | | | | Refer to Lab manual | | | | | | | |
| | | | | for exact timings | | | | | | | |
| Follow-up contact | | | | | | | | | | | X |
| for survival/ | | | | | | | | | | | |
| progression (n) | | | | | | | | | | | |

(a) Archival sample if available in Phase I, the dose escalation phase (retrospective MT1-MMp status). Tumor sample, whether archival or fresh, mandatory in Phase IIa, the expansion phase to confirm patient eligibility. Archival tumor biopsy sample can be obtained within six months prior to the first dose. Fresh screening tumor biopsy must be performed/obtained within eight weeks prior to first dose.
(b) Pregnancy test: For female patients of child bearing potential.
(c) Complete physical examination to be performed Pre-treatment then all subsequent examinations can be symptom-directed and only performed as clinically indicated.
(d) Screening weight can be used to calculate BSA for Cycle 1.
(e) Clinical laboratory assessments should be performed within the previous seven days of Cycle 1 Day 1 and prior to each planned BT1718 administration on Days 1, 4, 8, 11, 15, 18 and 22 of Cycle 1. From Cycle 2 onwards, laboratory assessments must be performed and checked pror to BT1718 administration on Days 1, 8 and 15 and thereafter if clinically indicated. After six cycles, the frequency of haematology and biochemistry assessments may decrease at the direction of the PI and Sponsor but, at a minimum, must be performed on Day 1 of each cycle. Laboratory tests may be performed up to 24 hours prior to BT1718 administration but results must be available and reviewed by the Investigator before BT1718 is given. On Day 1 of each cycle a full biochemistry assessment should be performed and on subsequent dosing dayys core biochemistry assessment should be performed.
(f) In the event of a Grade 4 neutropenia or Grade 4 thrombocytopenia a full blood count must be performed at least on Day 7 (neutropenia) and Day 5 (thrombocytopenia) after the onset of the event to determine if a dose limiting toxicity has occured. Continue close monitoring until resolution to Grade 3 or less.
(g) To be repeated before biopsy procedure if done.
(h) To be done Cycle 1 and 2, within 1 hour before and 1 hour (+15 mins) after BT1718 administration, then clinically indicated thereafter.
(i) ECGs should be performed before BT1718 administration, annd within 1 ½ hours (+15 mins) after BT1718 administration during Cycle 1 Day 1. On Cycle 2 Day 1 an ECG should be performed before or within 1 1/2 hours (+15 mins) after BT1718 administration. From Cycle 2 onwards ECG should be repeated to Day 1 every two cycles.
(j) Pre-treatment tumor bopsy; optional for patients enrolled in the dose escalation phase (Phase I, Stage 1 and 2); mandatory for patients in the expansion phase (Phase IIa, Parts A and B) if no archival sample available for MT1-MMP screening; mandatory for a minimum of six patients in the expansion phase (Phase IIa, Parts A and B). Post-treatment tumor biopsy; optional for patients in the dose escalation phase (Phase I, Stage 1 and ); mandatory in a minimum of six patients in the expansion phase (Phase IIa, Parts A and B) in Cycle 1 on Day 8 or 15 performed 4 +/− 2 hours post treatment. If unable to perform the biopsy during Cycle 1, this may be performed 4 +/− 2 hours post treatment during Cycle 2 on Day 8 or 15.
(k) Pharmacokinetic (PK) and pharmacodynamics sample collection-refer to the laboratory Manual for specific timings.
(I) Unless performed within previous 28 days or PD seen on previous study scan.
(m) Monthly follow-up required ONLY for thos AEs and SAEs considered drug-related (highly probable, probable, or possible) and presetn at off-study visit. Monthly follow-up to continue until resolution, return to baseline, stabilisation or patient starts another anti-cancer treatment.
(n) All patients should be followed up for first progression and for survival until the end of trial. For those no longer on treatment, the site trial team should check the status of the patient at least three monthly (through NHS/HSC electronic data records or by phone calls only if appropriate) to determine when PD occurs (if not already occured) and if the patient remains alive.
(o) Optional non-tumor biopsy sample may also be taken from the same patient having the pre and post treatment tumor biopsies (i.e. during Cycle 1 Day 8 or 15 or Cycle 2 Day 8 or 15 performed 4 +/− 2 hours) in the dose escalation phase (Phase I, Stage 1 and 2) and expansion phase (Phase IIa, Parts A and B).
(p) The timing of CT/MRI schedule may change in the expansion phase (Phase IIa) depending on tumor type.
(q) Temperature, blood pressure (seated or lying after 5 mins rest) and pulse should be performed before and then repeated 1 hr (+/−15 mins) after BT1718 administration.

Pharmacokinetic and Pharmacodynamic Assessments

Summary of PK and Pharmacodynamic Assessments

TABLE 12
Summary of PK and pharmacodynamic assessments

| | | | | | | |
|---|---|---|---|---|---|---|
| | | | | | Time points | Time points |
| | | | | | and | and |
| | | | | | approximate | approximate |
| | | Purpose of | Type of | Patient | volume | volume |
| Biomarker | Technology | assay/Rationale | sample | group | (once weekly) | (twice weekly) |

SECONDARY ENDPOINTS

| | | | | | |
|---|---|---|---|---|---|
| Pharmacokinetics | LC/MSMS | To determine | Blood | Phase I, dose | Up to 24 time points over the |
| ofBT1718 | | the PK profile | (plasma) | escalation | first two cycles. |
| | | ofBT1718. | | phase (Stage | Approximately 96 mL. |
| | | | | 1 and 2) and | |
| | | | | potentially in | |
| | | | | Phase IIa, | |
| | | | | expansion | |
| | | | | phase (Parts | |
| | | | | AandB) | |

TERTIARY ENDPOINTS

| Pharmacokinetics | LC/MSMS | Concentration | Urine sample | Phase I, dose | Urine collection post Cycle 1 | |
|---|---|---|---|---|---|---|
| ofBT1718 | | DMl-SHin | | escalation | Day 1 administration. | |
| | | urine, proof of | | phase (Stage | | |
| | | principle of | | 1 and 2) and | | |
| | | renal clearance. | | potentially in | | |
| | | | | Phase IIa, | | |
| | | | | expansion | | |
| | | | | phase (Parts | | |
| | | | | AandB) | | |
| Immunogenicity of | ELISA | To evaluate the | Blood | Phase I, dose | Pre-dose C1 D1, pre-dose C2 | |
| BT1718 | | potential of | (serum) | escalation | D1, pre-dose C3 D1 and at Off | |
| | | ADA against | | phase (Stage | study. | |
| | | BT1718. | | 1 and 2) and | Approximately 6 mL per sample. | |
| | | | | potentially in | | |
| | | | | Phase IIa, | | |
| | | | | expansion | | |
| | | | | phase (Parts | | |
| | | | | AandB) | | |
| MT1-MMP | IHC, in-situ | Correlate | Formalin- | Retrospective | NA | NA |
| expression | | expression with | fixed | analysis in | | |
| | | response, | paraffin- | Phase I | | |
| | | identify patients | embedded | (Stage 1 and | | |
| | | most likely to | (FFPE) | 2) and | | |
| | | respond | archival | prospective | | |
| | | (predictive | tumor | selection in | | |
| | | biomarker). | samples/fresh | Phase IIa, | | |
| | | | tumor | expansion | | |
| | | | samples | phase (Parts | | |
| | | | | AandB) | | |
| Markers of cell | IHC or | To demonstrate | FFPE tumor | Phase I, dose | 4 +/− 2 hours post treatment on | |
| death including but | ELISA | evidence of cell | and non- | escalation | Cycle 1 Day 8 or Cycle 1 Day | |
| not limited to p- | | death/inhibition | tumor | phase (Stage | 15 | |
| Histone H3, caspase | | of cell | samples and | 1 and 2) and | (if unable to perform biopsy | |
| 3, H2AX foci) and | | proliferation | fresh tumor | Phase IIa, | during Cycle 1, biopsy may be | |
| proliferation/growth | | mediated by | sample | expansion | performed during Cycle 2, Day 8 | |
| arrest (including but | | cytotoxic | lysates where | phase (Parts | or 15 (4 +/− 2hours post | |
| not limited to Ki67 | | payload of | possible (for | AandB) | treatment). | |
| and Aurora B) | | BT1718 (proof | ELISA) | | | |
| | | of principle); | | | | |
| | | compare results | | | | |
| | | with non-tumor | | | | |
| | | sample where | | | | |
| | | possible. | | | | |
| Markers of immune | IHC | Determine if | FFPE tumor | Phase IIa, | 4 +/− 2 hours post treatment on | |
| stimulatory | (multiplexed) | there is immune | samples | expansion | Cycle 1 Day 8 or Cycle 1 Day | |
| response post-cell | | cell infiltration | | phase (Parts | 15 | |
| death induced by | | to the tumor | | AandB) | (if unable to perform biopsy | |
| BT1718, IHC of | | | | | during Cycle 1, biopsy may be | |
| immune cells in | | | | | performed during Cycle 2, Day 8 | |
| tumor | | | | | or 15 (4 +/− 2hours post | |
| | | | | | treatment). | |
| Markers of | IHC | To understand | FFPE tumor | Phase IIa, | As above | |
| resistance | | resistance | samples | expansion | | |
| (including but not | | mechanism. | | phase (Parts | | |
| limited to Pgp, | | | | AandB) | | |
| FAK/pFAK, beta | | | | | | |
| tubulin) | | | | | | |
| Circulating | ELISA | Potential for | Blood | Phase IIa, | Day -7; pre-dose C1 D1; 24hrs | |
| biomarkers (may | | predictive | (plasma) | expansion | C1 D1; pre-dose Cl D8; pre- | |
| include but not | | biomarkers in | | phase (Parts | dose C1 D15; pre-dose C3 D1 | |
| limited to MMP14, | | the circulation. | | AandB) | (first response assessment scan); | |
| 2, TIMP-1, | | | | | and at Off study. | |
| Vascular | | | | | Approximately 5 mL per | |
| Endothelial Growth | | | | | sample. | |
| Factor | | | | | | |
| CTC | Parsotix | Explore | Blood | Phase IIa, | Pre-dose C1 D1, pre-dose C1 | |
| | | correlation | (plasma) | expansion | D8, pre-dose Cl D15, pre-dose | |
| | | between CTC | | phase (Parts | C3 D1; and at Off study. | |
| | | MT1 MMP | | AandB) | Approximately 10 mL per | |
| | | expression and | | | sample. | |
| | | tumor. Potential | | | | |
| | | prognostic | | | | |
| | | marker. | | | | |
| cfDNA | ddPCR | Prognostic | Blood | Phase I, dose | Pre-dose C1 D1, pre-dose C1 | |
| | | marker of | (plasma) | escalation | D8, pre-dose Cl D15 and pre- | |
| | | response, | | phase (Stage | dose C3 D1); and at Off study. | |
| | | potential to | | 1 and 2) and | Approximately 10 mL per | |
| | | correlate | | Phase IIa, | sample. | |
| | | response with | | expansion | | |
| | | mutational | | phase (Parts | | |
| | | burden, identify | | AandB) | | |
| | | responders vs | | | | |
| | | non-responders | | | | |
| | | and relapsers. | | | | |
| M30/M65 | ELISA | Measure | Blood | Phase I, dose | Pre-dose C1 | Pre-dose C1 |
| | | circulating cell | (serum) | escalation | D1, 24 hrs C1 | D1, 24 hrs C1 |
| | | death | | phase (Stage | D1, pre-dose | D1, pre-dose |
| | | biomarkers as a | | 1 and 2) and | C1 D8 and | C1 D4, pre- |
| | | result of tumor | | Phase IIa, | pre-dose C1 | dose C1 D8 |
| | | cell killing | | expansion | D15. | and pre-dose |
| | | (proof of | | phase (Parts | Approximately | C1 D15 |
| | | principle). | | AandB) | 3 mL per | Approximately |
| | | | | | sample. | 3 mL per |
| | | | | | | sample. |
| MT1-MMP | Flow | Determine if | PBMC | Phase IIa, | Pre-dose C1 | Pre-dose C1 |
| expression in | cytometry | expression of | | expansion | D1, pre-dose | D1, pre-dose |
| circulating Myeloid | | MT1 MMP in | | phase (Parts | C1 D8, pre- | C1 D4, pre- |
| cells (neutrophils, | | immune cells | | AandB) | dose C1 D15, | dose C1 D8, |
| monocytes, | | changes with | | | C3 D1); | pre-dose C1 |
| eosinophils and | | treatment and | | | Off study. | D15, pre-dose |
| basophils) and | | provide any | | | Approximately | C3 D1); |
| lymphoid (T, N, | | evidence for | | | 10 mL per | Off study. |
| NK) | | immune cell | | | sample. | Approximately |
| | | infiltration to | | | | 10 mL per |
| | | the tumor from | | | | sample. |
| | | circulation. | | | | |

Secondary Assessments

BT1718 Pharmacokinetics (Plasma)

Intact BT1718 and total DM1 (DM1 in BT1718, any peptidyl-DM1 metabolites of BT1718, and other DM1-containing mixed disulfides and free DM1) will be measured in plasma according to agreed standard operating procedures (SOPs) and validated methods in Phase I, dose escalation phase (Stage 1 and 2) and potentially in Phase IIa, expansion phase (Parts A and B), dependent on emerging data.

Tertiary/Research Assessments

MT1-MMP Expression in Tumor Tissues

A validated MT1-MMP prototype IHC analytical method will be developed to determine MT1-MMP expression according to agreed SOPs and validated methods. For Phase I, dose escalation phase (Stage 1 and 2), archival samples and optional pre and post biopsies will be used to measure MT1 MMP expression retrospectively. Prospective analysis of MT1-MMP will be done for the Phase IIa, expansion phase (Parts A and B) to select patients prior to trial entry. In addition, a minimum of six paired fresh biopsies will be mandated in each expansion arm (A and B) to investigate MT1-MMP expression levels pre vs post treatment.

Depending on emerging knowledge of target expression, biology and availability of tumor material, markers of immune cell infiltrates to the tumor may also be investigated using molecular histology techniques.

BT1718 Pharmacokinetics (Urine)

Total DM1 will be measured in urine post Cycle 1 Day 1 according to agreed SOPs and validated methods. Intact BT1718 will also potentially be measured in these urine samples dependent on emerging data.

Immunogenicity

Serum samples will be analysed for potential immunogenicity to BT1718 according to agreed SOPs and validated methods in Phase I, dose escalation phase (Stage 1 and 2) and potentially in Phase IIa, expansion phase (Parts A and B), dependent on emerging data.

Markers of Cytotoxicity in Tumor Samples and Non-Tumor Samples

Fresh tumor and non-tumor biopsies (where possible) from patients in Phase I, dose escalation phase (Stage 1 and 2) will be used to measure markers of cytotoxicity according to agreed SOPs and validated methods.

Fresh paired biopsies from at least six patients in each Phase IIa, expansion phase (Parts A and B) will be used to measure markers of cytotoxicity according to agreed SOPs and validated methods.

Markers of Resistance in Tumor Samples

Tumor biopsies will be used to measure markers of resistance in Phase IIa, expansion phase (Parts A and B) according to agreed SOPs and validated methods.

Circulating Biomarkers

Blood will be collected at specific time points pre and post treatment during Phase IIa, expansion phase (Parts A and B), according to agreed SOPs and validated methods.

Circulating Tumor Cells

Circulating tumor cells will be measured in blood in Phase IIa, expansion phase (Parts A and B) according to agreed SOPs and validated methods. Circulating tumor cells may be stored and analyzed at the end of the study.

Cell Free DNA

Cell free DNA will be measured in plasma from patients in Phase I, dose escalation phase (Stage 1 and 2) and Phase IIa, expansion phase (Parts A and B) according to agreed SOPs and validated methods.

Circulating Immune Cells

MT1-MMP expression and other expression markers may be evaluated in a range of circulating immune cells known to express MT1-MMP in Phase IIa, expansion phase (Parts A and B), according to agreed SOPs.

Circulating Cell Death Markers—M30 and M65

Samples for cell death markers will be taken from patients in the trial. M30 and M65 ELISA assays will be used to measure markers of cell death in serum in Phase I, dose escalation phase (Stage 1 and 2) and Phase IIa, expansion phase (Parts A and B) according to agreed SOPs and validated methods.

Assessment of Safety

Investigator Responsibilities

The investigator is responsible for monitoring the safety of patients who have enrolled in the trial and for accurately documenting and reporting information as described in the following sections.

Adverse Event Definitions

Adverse Event

An AE is any untoward, undesired or unplanned medical occurrence in a patient administered an investigational medicinal product (IMP), a comparator product or an approved drug.

An AE can be a sign, symptom, disease, and/or laboratory or physiological observation that may or may not be related to the IMP or comparator.

An AE includes but is not limited to those in the following list.

A clinically significant worsening of a pre-existing condition. This includes conditions that may resolve completely and then become abnormal again.

AEs occurring from an overdose of an IP, whether accidental or intentional.

AEs occurring from lack of efficacy of an IMP, for example, if the Investigator suspects that a drug batch is not efficacious or if the Investigator suspects that the IMP has contributed to disease progression.

Serious Adverse Events

A serious adverse event is any AE, regardless of dose, causality or expectedness, that:

results in death;

is life-threatening*;

requires in-patient hospitalization or prolongs existing in-patient hospitalization (some hospitalizations are exempt from SAE reporting—e.g. hospital admissions planned prior to the patient entering the trial; overnight stays for planned procedures such a blood transfusions);

results in persistent or significant incapacity or disability;

is a congenital anomaly or birth defect;

is any other medically important event.**

* A life-threatening event is defined as an event when the patient was at substantial risk of dying at the time of the adverse event, or use or continued use of the device or other medical product might have resulted in the death of the patient

**A medically important event is defined as any event that may jeopardize the patient or may require intervention to prevent one of the outcomes listed above. Examples include allergic bronchospasm (a serious problem with breathing) requiring treatment in an emergency room, serious blood dyscrasias (blood disorders) or seizures/convulsions that do not result in hospitalization. The development of drug dependence or drug abuse would also be examples of important medical events

For fatal SAEs, wherever possible report the cause of death as an SAE with a fatal outcome rather than reporting death as the SAE term. When available the autopsy report will be provided to the Sponsor.

Any dose DLT must be reported to the Sponsor's CSM and CRA within 24 hours of site staff becoming aware of the DLT. The Sponsor's Pharmacovigilance Department must be copied into any initial or follow up email notification.

Other reportable events that must be treated as SAEs are listed below.

Overdose with or without an AE.

Inadvertent or accidental exposure to an IMP with or without an AE, including for example, spillage of the IMP that contaminates staff.

Any AE that could be related to the protocol procedures, and which could modify the conduct of the trial.

Events of pregnancy must be reported and treated in the same way as SAEs:

Pregnancy. Any pregnancy occurring in a patient or a patient's partner during treatment with an IMP or occurring within six months of the last IMP administration, must be reported to the Pharmacovigilance Department on a pregnancy report form in the same timelines as an SAE. These should be reported even if the patient is withdrawn from the trial.

If during the course of the study, other medically important events are identified and there is a requirement to report specific events outside of the standard criteria, this will be communicated to site and the protocol will be updated to reflect this.

Suspected, Unexpected, Serious Adverse Reactions

A SUSAR is a suspected, unexpected, serious adverse reaction. All AEs and SAEs will be assessed by the sponsor for seriousness, causality and expectedness. The Pharmacovigilance Department will expedite all SUSARs to the relevant Competent Authority/Authorities and the relevant REC(s) within the timelines specified in legislation (SI 2004/1031 as amended).

Determining Adverse Event Causality

The relationship of an AE to the BT1718 is determined as follows.

Highly Probable

Starts within a time related to the IMP administration and

No obvious alternative medical explanation.

Probable

Starts within a time related to the IMP administration and

Cannot be reasonably explained by known characteristics of the patient's clinical state.

Possible

Starts within a time related to the IMP administration and

A causal relationship between the IMP and the AE is at least a reasonable possibility.

Unlikely

The time association or the patient's clinical state is such that the trial drug is not likely to have had an association with the observed effect.

Not Related

The AE is definitely not associated with the IMP administered.

Note: Drug-related refers to events assessed as possible, probable or highly probable.

The Investigator must endeavor to obtain sufficient information to determine the causality of the AE (i.e. IMP, other illness, progressive malignancy etc) and must provide his/her opinion of the causal relationship between each AE and IMP. This may require instituting supplementary investigations of significant AEs based on their clinical judgement of the likely causative factors and/or include seeking a further opinion from a specialist in the field of the AE.

The following guidance should be taken in to account when assessing the causality of an AE:

Previous experience with the IMP and whether the AE is known to have occurred with the IMP.

Alternative explanations for the AE such as concomitant medications, concurrent illness, non-medicinal therapies, diagnostic tests, procedures or other confounding effects.

Timing of the events between administration of the IMP and the AE.

IMP blood levels and evidence, if any, of overdose.

De-challenge, that is, if the IMP was discontinued or the dosage reduced, what happened to the adverse reaction?

Re-challenge, that is, what happened if the IMP was restarted after the AE had resolved?

Assessing the causality of an AE should be based on the information that is available at the time of reporting.

Expectedness

Assessment of expectedness for BT1718 will be made by the Pharmacovigilance Department against the current version of the IB.

Collection of Safety Information

Screening Failures

For patients who fail screening, SAEs must be reported to the Sponsor's Pharmacovigilance Department, from the date of consent until the date the patient has been confirmed as ineligible.

Eligible Patients

For eligible patients, SAE and AE collection and monitoring will commence at the time the patient provides written consent to participate in the trial and will continue until 28 days after the last administration of BT1718 or until the patient starts another anti-cancer therapy.

Should an Investigator become aware of any drug-related SAEs after this 28 day period, these must also be reported to the Sponsor within the expedited timelines.

Follow-Up of AEs and SAEs

Follow-up of AEs with a causality of possible, probable or highly probable will continue until the events resolve, stabilize or the patient starts another anti-cancer therapy.

The Pharmacovigilance Department will make requests for further information on SAEs to the trial site at regular intervals. Requested follow-up information should be reported to the Pharmacovigilance Department within 24 hours of first becoming aware of the follow up information. For fatal or life threatening cases, follow-up information must be sought and reported to the Pharmacovigilance Department as soon as becoming aware.

Other Safety Information of Interest

We will also collect information on the following situations, whether they are associated with an AE or not:

Abuse or Misuse

Any occurrences of these should be reported in the same manner as SAEs.

Reporting of SAEs to the Sponsor's Pharmacovigilance Department

All SAEs, regardless of causality, must be reported to the Pharmacovigilance Department in an expedited manner.

SAEs should be documented on an SAE report form, using the completion guidelines provided.

Each episode of an SAE must be recorded on a separate SAE report form. The NCI CTCAE Version 4.02 must be used to grade the severity of each SAE, and the worst grade recorded. If new or amended information on a previously reported SAE becomes available, the Investigator should report this to the Pharmacovigilance Department on a new SAE report form.

If the SAE has not been reported within the specified timeframes, a reason for lateness must be added on the form when sending the SAE report form to the Pharmacovigilance Department.

Should the Investigator become aware of any drug-related SAEs after the patient goes “off-study”, these must also be reported to the Pharmacovigilance Department within the specified timelines specified above

Events exempt from being reported as SAEs to the Pharmacovigilance Department

Events specified in this section do not require reporting as SAEs in this trial, unless hospitalization is prolonged for any reason and then an SAE form must be completed. The events must still be recorded in the appropriate section of the eCRF.

Elective admissions—Elective admissions to hospital for procedures which were planned prior to entering the trial are not SAEs. Hospitalization for administration of the IMP according to the trial protocol is also exempt from being reported as an SAE.

Death due to disease progression—Cases of death due to disease progression do not require SAE reporting, unless considered related to the IMP.

Recording of Adverse Events and Serious Adverse Events in eCRFs

All AEs, including SAEs, must be recorded in the eCRF for eligible patients. All concomitant medications, including herbal medications and supplements must be recorded. Any therapy used to treat the event must be recorded. The eCRF will be reconciled with the safety database during and at the end of the trial. Therefore, the sites should ensure the data entered on the paper SAE report form (which is used by the Pharmacovigilance Department only) and the data entered into the eCRF are consistent. The Sponsor's Medical Advisor and the Investigator(s) will regularly review the safety data from both the safety and the clinical database.

Urgent Safety Measures

The Sponsor or Investigator may take appropriate urgent safety measures (USMs) in order to protect the patient of a clinical trial against any immediate hazard to their health or safety. This includes procedures taken to protect patients from pandemics or infections that pose serious risk to human health.

USMs may be taken without prior authorization from the competent authority.

The MHRA and the REC must be notified within three days of such measures being taken.

Should the site initiate a USM, the Investigator must inform the Sponsor immediately.

The Notification Must Include:

the date of the USM;

who took the decision; and

why action was taken.

The Sponsor will then notify the MHRA and the REC within three days of USM initiation.

Assessment of Efficacy

Measurement of Disease

Disease must be measured according to the RECIST v1.1 criteria given in Appendix 2.

Timing and Type of Tumor Assessments

A thorough clinical and radiological evaluation of malignancy, as judged appropriate by the Investigator, and in line with the protocol, must be performed before a patient receives their first dose of BT1718. The same methods that detect evaluable lesions at baseline must be used to follow these lesions throughout the trial. To ensure compatibility, the radiological assessments used to assess response must be performed using identical techniques. Imaging based evaluation is preferred to evaluation by clinical examination when both methods have been used to assess the anti-tumor effect of a treatment.

All radiological assessments must be performed within four weeks (28 days) before starting treatment with BT1718. The interval between the last anti-cancer therapy and these measurements must be at least four weeks. All clinical measurements to assess response must be performed within one week of the first dose of BT1718.

Complete responses (CR) and partial responses (PR) need to be confirmed by a subsequent assessment at least four weeks later. Stable disease criteria must be met at least once after study entry at a maximum interval of six weeks to be defined as SD. There is no requirement for repeat assessments to be performed in order for the patient to be assigned a status of CR or PR.

Copies of the scans must be available for external independent review if requested by the Sponsor.

Baseline Evaluations

These must include radiological measurements of lesions in the chest, abdomen, and pelvis by CT scan or MRI scan and/or other radiological measurements as clinically indicated or clinical measurements as appropriate e.g. assessment of palpable lesions or measurement of tumor markers. All areas of disease present must be documented (even if specific lesions are not going to be followed for response) and the measurements of all measurable lesions must be recorded clearly on the scan reports. Any non-measurable lesions must be stated as being present. For clinical measurements, documentation by colour photography including a ruler to estimate the size of the lesion is strongly recommended, as this aids external independent review of responses (See Appendix 2).

Evaluations During and at ‘Off-Study’

Tumor assessments must be repeated every two cycles (+/−7 days) or more frequently, when clinically indicated. All lesions measured at baseline must be measured at every subsequent disease assessment, and recorded clearly on the scan reports. All non-measurable lesions noted at baseline must be noted on the scan report as present or absent.

All patients, who are removed from the trial for reasons other than PD, must be re-evaluated at the time of treatment discontinuation, unless a tumor assessment was performed within the previous four weeks.

It is the responsibility of the Principal Investigator to ensure that the radiologists are aware of the requirement to follow-up and measure every target lesion mentioned at baseline and comment on the non-target lesions in accordance with RECIST 1.1 criteria.

Tumor Response

All patients who meet the eligibility criteria and receive at least one cycle of trial medication and have a baseline and at least one post-baseline assessment of disease will be evaluable for response. Patients who develop clear evidence of PD without a formal disease assessment will be considered non responders. Confirmatory repeat assessments are required at least four weeks after an initial indication of CR or PR, in order for the patient to be assigned a status of confirmed CR or PR. To be assigned a status of SD, follow-up measurements must have met the SD criteria at least once and at least six weeks after the initial dose of BT1718 is given.

Should rapid tumor progression occur before the completion of four weeks the patient will be classified as having early progression.

Tumor response should be classified as “not evaluable”, only when it is not possible to classify it under another response category, for example, when baseline and/or follow-up assessment is not performed or not performed appropriately.

Expert reviewers appointed by the Sponsor may undertake an independent review of the Investigator's assessed objective responses (CR and PR). The expert reviewers will include at least one specialist who is not an Investigator in the study. Any independent reviewer's assessment will also be documented in the final CSR along with the assessment made by the Investigator. The eCRF will reflect the Investigator's opinion.

Recording of Response in the eCRF

The applicable overall response category for each visit that includes disease assessment must be recorded in the eCRF.

Other Definitions of Outcome

Toxic death: Any death to which drug toxicity is thought to have a major contribution.

Early death: Death during the first 28 days of treatment.

Patient Withdrawal Before Completion of Treatment Schedule

The Investigator must make every reasonable effort to keep each patient on trial for the whole duration of the trial (i.e. until 28±7 days after last BT1718 administration). However, if the Investigator removes a patient from the trial or if the patient declines further participation, final ‘off study’ assessments should be performed ideally before any subsequent therapeutic intervention. All the results of the evaluations and observations, together with a description of the reasons for withdrawal from the trial, must be recorded in the medical records and in the eCRF.

Patients who are removed from the trial due to adverse events (clinical or laboratory) will be treated and followed according to accepted medical practice. All pertinent information concerning the outcome of such treatment must be recorded in the eCRF and on the serious adverse event (SAE) report form where necessary.

The following are justifiable reasons for the Investigator to withdraw a patient from the trial.

Adverse Event (AE)/SAE;

Withdrawal of Consent;

Serious deviation from the trial protocol (including persistent patient attendance failure and persistent non-compliance);

Sponsor's Decision to Terminate the Trial;

Withdrawal by the Investigator for Clinical Reasons not Related to BT1718;

Evidence of Disease Progression;

Pregnancy (for Female Patients During the Study)

Defining the End of Trial

The ‘end of trial’ is defined as the date when the last patient has completed the ‘off-study’ visit or the final follow-up visit (whichever is the latter). The ‘off-study’ visit is scheduled to take place 28+/−7 days after the last dose of BT1718 administration.

IMP will be available for two years from the latest date that any remaining patients started treatment. After this, any remaining patients will come off-study and the trial will end as above.

The end of trial cannot be declared while any patient is still having study visits or while study data is still being collected e.g. progression-free or OS data.

If an arrangement becomes possible where patients can continue on prolonged or extended use of IMP, beyond the end of trial, that arrangement will be distinct from the trial and will not prevent the end of trial being declared.

It is the responsibility of the CDD to inform the MHRA and the REC within 90 days of the ‘end of the trial’ that the trial has closed.

In cases of early termination of the trial (for example, due to toxicity) or a temporary halt by the CDD, the CDD will notify the MHRA and the REC within 15 days of the decision and a detailed, written explanation for the termination/halt will be given.

Recruitment Will Cease when:

The drug is considered too toxic to continue treatment before the required number of patients have been recruited.

The stated number of patients to be recruited has been reached.

The stated objectives of the trial are achieved.

Regardless of the reason for termination, all data available for patients at the time of discontinuation of follow-up must be recorded in the eCRF. All reasons for discontinuation of treatment must be documented.

In terminating the trial, the Sponsor and the Investigators must ensure that adequate consideration is given to the protection of the patient's interest.

Data Analysis and Statistical Considerations

The final analysis will be conducted after one of the following conditions is met:

The trial is terminated early

The end of trial has been reached.

Sample Size

Phase I, Dose Escalation Phase

The number of patients required for the phase I will depend on the number of dose levels required to be explored to determine the MTD. It is anticipated that approximately 50 to 60 patients will be entered between Stages 1 and 2, the final number will depend on the number of dose escalations required and the number of evaluable patients.

Phase IIa, Expansion Phase

Part A and Part B—14 patients will be recruited into both part A and B to further characterise the toxicity profile and tolerability of the RP2D. A total of 14 patients would also allow the detection of a response rate of 30% and exclusion of a response rate of 10% with 80% power and significance level of 0.2.

Part C & D—For cohorts of TNBC or NSCLC, using Simon 2-stage design with power=80% and α=0.2, a maximum of 15 patients are required to detect a desirable response rate of 30% and exclude a response rate as low as 10%. An interim analysis will be carried out after 6 patients, if at least 1 response is seen at four months recruitment will continue up to a total of 15 patients. If no responses are seen the cohort will stop for futility. If 3 or more responses are observed out of 15 patients we would conclude that the response rate is not <10% and is more likely to be ≥30%.

For a cohort of sarcoma patients, using a Simon 2-stage design with power=80% and α=0.2, a maximum of 16 patients are required to detect a desired clinical benefit rate of 40% and exclude a clinical benefit rate as low as 20%. An interim analysis will be carried out after 11 patients, if at least 3 responses/disease stabilizations are seen at three months recruitment will continue up to a total of 16 patients. If fewer than 3 responses/disease stabilizations are seen the cohort will stop for futility. If 5 or more responses or disease stabilizations are seen out of 16 patients we would conclude that the clinical benefit rate is not <20% and is more likely to be ≥40%.

It is therefore anticipated that approximately 15-16 patients will be required per cohort in parts C/D however the final tumor types to be recruited to part C and D may be informed by the results of part A/B or external evidence available at the time of commencement of the phases of the trial.

Presentation of Data

Data will be presented in a descriptive fashion. Variables will be analyzed to determine whether the criteria for the trial conduct are met. This will include a description of patients who did not meet all the eligibility criteria, an assessment of protocol deviations, IMP accountability and other data that impact on the general conduct of the trial.

Baseline characteristics will be summarized for all enrolled patients. Patients who died or withdrew before treatment started or did not complete the required safety observations will be described and evaluated separately.

Treatment administration will be described for all cycles. Dose administration, dose modifications or delays and the duration of therapy will be described.

Safety

Safety data will be collected from the date of written consent. Safety variables will be summarized by descriptive statistics. Laboratory variables will be described using NCI CTCAE Version 4.02.

Adverse events will be reported for each dose level and presented as tables of frequency of AEs by body system and by worse severity grade observed. Tables should indicate related and unrelated events.

Pharmacokinetics

The plasma concentration/time data will be analyzed using non-compartmental methods. The PK parameters to be determined for intact BT1718 include Cₘₐₓ, Tₘₐₓ, AUC, t_{1/2}, total body clearance (CL_{T}) and Vd_{SS}. The PK parameters to be determined for total DM1 include Cₘₐₓ, Tₘₐₓ, AUC and t_{1/2}.

Total DM1-SH will be measured in urine collected over 24 hours post first dose to determine percentage of DM1 excreted in urine. In addition, intact BT1718 may also be assessed.

Immunogenicity assessments will also be undertaken for BT1718 and reported as positive or negative.

Pharmacodynamics

The pharmacodynamic analyses and reports will undergo a quality control step prior to finalisation and will be signed by the person responsible for performing the assays and where appropriate the laboratory QA manager.

The anticipated data analysis paradigms may be subject to change throughout the course of this study. As a result, data will be analysed as agreed between lab and sponsor for each assay, following development and validation of each assays to agreed SOP.

Data will be reported in a format and timeframe as agreed between the lab and the Sponsor.

All pharmacodynamic samples collected will be analysed as described above.

Anti-Tumor Activity

Documenting anti-tumor activity is a secondary objective of this trial. Patients must receive at least one cycle of the trial medication to be evaluable for response. Objective responses, the best tumor response achieved by each patient while on trial and the time to progression will be presented in the data listings by cohort.

The response rate (proportion of evaluable patients with objective response) will be reported by cohort. Progression free survival will be calculated from trial entry until the time of documented disease progression or death (whichever occurs first). Patients who are alive and progression free or lost to follow up at the time of analysis will be censored at the time the patient was last known to be alive and progression free. Overall survival will be calculated from trial entry until the time of death from any cause. Patients who are alive or lost to follow up at the time of analysis will be censored at the time the patient was last known to be alive. Duration of response will be measured from the date of the first scan where response was seen until date of first radiographical progression or death. Median PFS, OS and duration of response will be presented. The PFS and OS rate at 6 months will also be presented. 95% confidence intervals will be reported.

Administration

This trial is conducted under a clinical trial authorisation and approval from the MHRA and the relevant REC(s) will be obtained before the start of this trial. This trial is sponsored and monitored by the Cancer Research UK, CDD. Applicable regulatory requirements are described in this section.

Protocol Deviations and Amendments

The protocol should be adhered to throughout the conduct of the study, if a situation arises where the conduct of the study may not be in line with the protocol, then site should contact the CDD to discuss this.

Amendments to the protocol may only be made by the Sponsor. A protocol amendment may be subject to review by the assigned Ethics Committee, HRA and the MHRA. Written documentation of the Ethics Committee and HRA (and if appropriate the MHRA) ‘favorable opinion’ (i.e. approval) must be received before the amendment can be implemented and incorporated into the protocol if necessary.

Serious Breach of GCP

A serious breach is a breach which is likely to effect to a significant degree: the safety or physical or mental integrity of the subjects of the trial, or the scientific value of the trial.

In order that the Sponsor can fulfil their obligations in terms of reporting serious breaches of GCP to the MHRA within seven calendar days of identification, site staff must inform the Sponsor of any unplanned deviations to the trial protocol (or GCP principles) as soon as possible after the deviation occurs to allow prompt evaluation by the Sponsor.

Completion of the Electronic Case Report Form (eCRF)

Electronic CRFs approved by the Sponsor will be used to collect the data. The Investigator is responsible for ensuring the accuracy, completeness, clarity and timeliness of the data reported in the eCRFs.

Only the Investigator and those personnel who have signed the Delegation Log provided by the Sponsor and have been authorized by the Investigator should enter or change data in the eCRFs. Authorized users will be included on a user list in order to be provided access to the eCRF. All protocol required investigations must be reported in the eCRF. The Investigators must retain all original reports, traces and images from these investigations for future reference.

The collection and processing of personal data from the patients enrolled in this clinical trial will be limited to those data that are necessary to investigate the efficacy, safety, quality and usefulness of BT1718 used in this trial. The data must be collected and processed with adequate precautions to ensure patient confidentiality and compliance with applicable data privacy protection according to the applicable regulations. The data collected will comply with Directive 95/46/EC of the European Parliament and of the Council of 24 Oct. 1995 on the protection of individuals with regard to the processing of personal data.

Data will be entered directly into eCRF by authorized site personnel. Amendments to eCRF data will be made directly to the system and the system audit trail will retain details of the original value(s), who made the change, a date and time, and a reason for the change.

Once an eCRF form has been entered by the site personnel, the data are cleaned using manual and automated checks. Queries will be issued electronically to the site. Authorized personnel must answer the queries by making relevant amendments to data or providing a response. Answered queries will be closed or reissued as appropriate.

Once the patient is ‘off study’ and the eCRF has been fully completed, the Investigator must provide an electronic signature to authorise the complete subject casebook.

At the end of the trial all eCRFs are retained and archived by the Sponsor and a PDF copy provided to the Investigator who is responsible for archiving at site.

Trial Performance, Monitoring, Auditing and Inspection

Before the trial can be initiated, the prerequisites for conducting the trial must be clarified and the organizational preparations made with the trial centre. The sponsor must be informed immediately of any change in the personnel involved in the conduct of the trial.

During the trial the Sponsor's CRA will be responsible for monitoring data quality in accordance with their SOPs. A strategic monitoring approach, including targeted source data verification, will be implemented where appropriate.

Before the study start, the Investigator will be advised of the anticipated frequency of the monitoring visits. The Investigator will receive reasonable notification before each monitoring visit.

It is the responsibility of the CRA to:

review trial records and compare them with source documents;

check pharmacokinetic and pharmacodynamic samples and storage;

discuss the conduct of the trial and the emerging problems with the Investigator;

check that the drug storage, dispensing and retrieval are reliable and appropriate; and

verify that the available facilities remain acceptable.

At the end of the trial all unused BT1718 supplied must be destroyed at site (only once authorized to do so by the CRA or CSM).

It is the responsibility of the Sponsor to notify the REC of the ‘end of the trial’.

During the course of the trial, the Quality Assurance Department of the CDD, or external auditors contracted by the CDD, may conduct an on-site audit visit.

Principal Investigators conducting this trial will accept the potential for inspection by the MHRA.

Source Document Verification

Unless agreed in writing, all data collected in the eCRF must be verifiable by the source data. Therefore, it is the Investigator's responsibility to ensure that both he/she and his/her study team records all relevant data in the medical records. The Investigator must allow the CRA direct access to relevant source documentation for verification of data entered into the eCRF, taking into account data protection regulations. Entries in the eCRF will be compared with patients' medical records and the verification will be recorded in the eCRF.

Some source data may exist only electronically and be entered, or loaded directly into the eCRF.

The patients' medical records, and other relevant data, may also be reviewed by appropriate qualified personnel independent from the Sponsor appointed to audit the trial, NHS Trust staff and by regulatory authorities. Details will remain confidential and patients' names will not be recorded outside the hospital.

Clinical Study Report

At appropriate intervals, interim data listings will be prepared to give the Investigator the possibility to review the data and check the completeness of information collected. All clinical data will be presented at the end of the trial on final data listings. The sponsor will prepare a clinical study report based on the final data listings. The report will be submitted to the Investigator(s) for review and confirmation it accurately represents the data collected during the course of the trial. Summary results of the trial will be provided by the Sponsor to the MHRA and to the REC.

Record Retention

During the clinical trial and after trial closure the Investigator must maintain adequate and accurate records to enable both the conduct of a clinical trial and the quality of the data produced to be evaluated and verified. These essential documents (as detailed in Chapter V of Volume 10 (Clinical Trials) of The Rules Governing Medicinal Products in the European Union based upon Section 8 of the ICH GCP Guidelines), E6 (R2) including source documents such as scans, trial related documents and copies of the eCRFs, associated audit trail and SAE report forms, shall show whether the Investigator has complied with the principles and guidelines of GCP.

All essential documents required to be held by the Investigator must be stored in such a way that ensures that they are readily available, upon request, to the Regulatory Agency or Sponsor, for the minimum period required by national legislation or for longer if needed by the sponsor. Records must not be destroyed without prior written approval from the Sponsor.

The medical files of trial subjects shall be retained in accordance with national legislation and in accordance with the maximum period of time permitted by the hospital, institution or private practice.

Ethical Considerations

Before starting the trial, the protocol and ICD must go through the CDDs external review process, and be approved by the Protocol and Safety Review Board and receive the favorable opinion of the assigned REC.

It is the Chief/Principal Investigator's responsibility to update patients (or their authorized representatives, if applicable) whenever new information (in nature or severity) becomes available that might affect the patient's willingness to continue in the trial. The Chief/Principal Investigator must ensure this is documented in the patient's medical notes and the patient is re-consented.

The Sponsor and Chief/Principal Investigator must ensure that the trial is carried out in accordance with the GCP principles and requirements of the UK Clinical Trials regulations (SI 2004/1031 and SI 2006/1928 as amended), the ICH GCP guidelines and the WMA Declaration of Helsinki—Ethical Principles for Medical Research Involving Human Subjects adopted by the 18th WMA General Assembly, Helsinki, Finland, June 1964 and all subsequent amendments including October 2013.

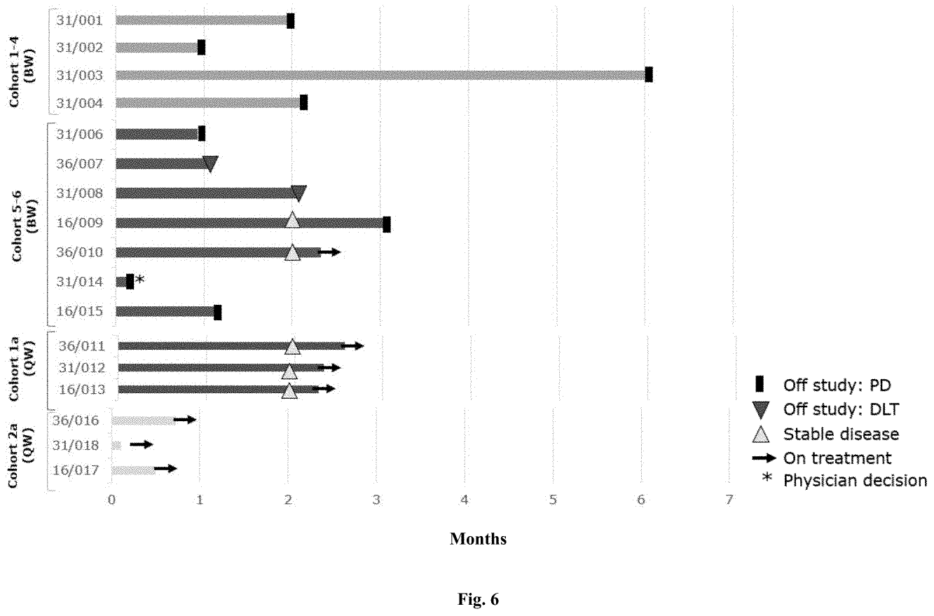

Patient status during BT1718 treatment is shown in Table A below and in FIG. 6.

TABLE A
Patient status during BT1718 treatment

| | Treatment | | Cut | Duration | | | Status as of |
|---|---|---|---|---|---|---|---|
| Patient No | Start | Off Study | off | Days | Patient | Months | 2 Jan. 2019 |
| 31/001 | 12 Feb. 2018 | 13 Apr. 2018 | | 61 | 31/001 | 2.03 | off study - disease |
| | | | | | | | progression |
| 31/002 | 13 Mar. 2018 | 12 Apr. 2018 | | 29 | 31/002 | 0.97 | off study - disease |
| | | | | | | | progression |
| 31/003 | 24 Apr. 2018 | 26 Oct. 2018 | | 182 | 31/003 | 6.07 | off study disease |
| | | | | | | | progression |
| 31/004 | 12 Jun. 2018 | 17 Aug. 2018 | | 65 | 31/004 | 2.17 | off study - disease |
| | | | | | | | progression |
| 31/005 | | | | | 31/005 | 0.00 | screen failure |
| 31/006 | 24 Jul. 2018 | 22 Aug. 2018 | | 28 | 31/006 | 0.93 | off study - disease |
| | | | | | | | progression - |
| | | | | | | | replaced as non- |
| | | | | | | | evaluable |
| 36/007 | 7 Aug. 2018 | 10 Sep. 2018 | | 33 | 36/007 | 1.10 | off study DLT |
| 31/XXX | | | | | 31/XXX | 0.00 | screen failure - no |
| | | | | | | | patient number |
| | | | | | | | assigned |
| 31/008 | 28 Aug. 2018 | 31 Oct. 2018 | | 63 | 31/008 | 2.10 | off study, disease |
| | | | | | | | progression |
| 16/009 | 3 Sep. 2018 | 4 Dec. 2018 | | 91 | 16/009 | 3.03 | off study, disease |
| | | | | | | | progression |
| 36/010 | 22 Oct. 2018 | ongoing | 2 | 70 | 36/010 | 2.33 | ongoing, stabile |
| | | | Jan. | | | | disease |
| | | | 2019 | | | | |
| 36/011 | 15 Oct. 2018 | ongoing | 2 | 77 | 36/011 | 2.57 | ongoing, stabile |
| | | | Jan. | | | | disease |
| | | | 2019 | | | | |
| 31/012 | 22 Oct. 2018 | ongoing | 2 | 70 | 31/012 | 2.33 | ongoing, stabile |
| | | | Jan. | | | | disease |
| | | | 2019 | | | | |
| 16/013 | 24 Oct. 2018 | ongoing | 2 | 68 | 16/013 | 2.27 | ongoing, stabile |
| | | | Jan. | | | | disease |
| | | | 2019 | | | | |
| 31/014 | 29 Oct. 18 | 14 Nov. 2018 | | 15 | 31/014 | 0.50 | off study, |
| | | | | | | | physician decision |
| 16/015 | 12 Nov. 2018 | 17 Dec. 2018 | | 35 | 16/015 | 1.17 | off study, |
| | | | | | | | progressive |
| | | | | | | | disease |
| 36/016 | 10 Dec. 2018 | ongoing | 2 | 22 | 36/016 | 0.73 | patient has |
| | | | Jan. | | | | commenced |
| | | | 2019 | | | | treatment as of |
| | | | | | | | 10 Dec. 2018 |
| 16/017 | 17 Dec. 2018 | ongoing | 2 | 15 | 16/017 | 0.50 | patient has |
| | | | Jan. | | | | commenced |
| | | | 2019 | | | | treatment as of |
| | | | | | | | 17 Dec. 2018 |
| 31/OXX (patient | 3 Jan. 2019 | ongoing | 2 | | 31/OXX | 0.00 | screening |
| number | | | Jan. | | | | planned for |
| assigned once | | | 2019 | | | | 31 Dec. 2018/prop |
| randomized - | | | | | | | osed treatment |
| will likely be | | | | | | | start first week |
| 31/018) | | | | | | | Jan. 2019 |

Definitions of disease status is in accordance with RECIST (Response Evaluation Criteria in Solid Tumors) 1.1, based on documentation of target and non-target lesions as specified in the BT1718 study protocol. All clinical measurements to assess response must be performed within one week of the first dose of BT1718.

Complete responses (CR) and partial responses (PR) need to be confirmed by a subsequent assessment at least four weeks later. Stable disease criteria must be met at least once after study entry at a maximum interval of six weeks to be defined as SD. Should rapid tumour progression occur before the completion of four weeks the patient will be classified as having early progression.

| Evaluation of Target Lesions | Evaluation of Non-Target Lesions |
|---|---|
| Complete Response (CR): Disappearance of all target | Complete Response (CR): Disappearance of |
| lesions for a period of at least one month | all non-target lesions and normalization of |
| | tumor marker levels |
| Complete Response Unknown (CRU): Complete | |
| response with persistent imaging abnormalities of | |
| unknown significance | |
| Partial Response (PR): At least a 30% decrease in the | |
| sum of the longest diameter of measured lesions | |
| (target lesions), taking as reference the baseline sum | |
| of the longest diameter | |
| Stable Disease (NR/SD): Neither sufficient shrinkage | Incomplete Response or Stable Disease (SD): |
| to qualify for PR nor sufficient increase to qualify for | Persistence of one or more non-target |
| PD, taking as reference the smallest sum of the | lesion(s) or/and maintenance of tumor |
| longest diameter since the treatment started | marker level above the normal limits |
| Progressive Disease (PD): A 20% or greater increase in | Progressive Disease (PD): Appearance of one |
| the sum of the longest diameter of measured lesions | or more new lesions and/or unequivocal |
| (target lesions), taking as reference the smallest sum | progression of existing non-target lesions |
| LD recorded since the treatment started or the | |
| appearance of one or more new lesions | |

An open label, first in human phase I/IIa study of once-weekly (QW) and twice-weekly (BIW) dosing schedules in patients with advanced solid tumours was undertaken.

4-week cycle: 1 hour intravenous (IV) infusions for 3 weeks, followed by a 1 week break.

### Trial Objectives:

Primary
Propose recommended Phase II dose (RP2D) by establishing the maximum tolerated dose (MTD) and maximum administered dose (MAD) of one or both dosing schedules (Phase I)
Safety and tolerability profile of BT1718 (Phase I/IIa)

Secondary
Investigate pharmacokinetics (PK) of BT1718 in humans (Phase I)
Assess preliminary signals of BT1718 efficacy, in MT1-MMP-expressing tumors (Phase IIa)

Tertiary
Explore potential predictive and pharmacodynamic biomarkers, including DM1 tumor levels.

A summary of the patient characteristics in the open label, first in human phase I/IIa study are shown in Table B, below.

TABLE B
Patient Characteristics

| | BIW | QW | | | |
|---|---|---|---|---|---|
| Characteristics | Patients | Patients | Tumour type | BIW | QW |
| No. of patients | 15 | 13 | Gastrointestinal (lower) | 1 | 0 |
| | | | Gastrointestinal (upper) | 3 | 0 |
| Male/Female | 8/7 | 6/7 | Genitourinary | 2 | 1 |
| | | | Gynecological | 4 | 3 |
| Median age | 56 | 59 | Head & neck | 0 | 2 |
| (range) | (27-72) | (22-77) | Sarcoma | 3 | 3 |
| | | | Skin | 1 | 1 |
| | | | Thoracic | 1 | 3 |

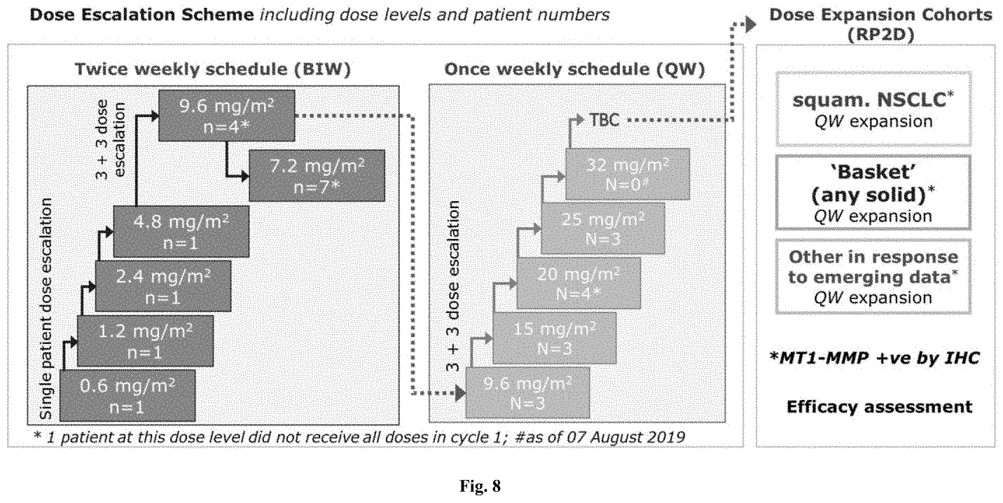

The Dose Escalation Scheme including dose levels and patient numbers is provided in FIG. 8.

Safety and Efficacy Results

Two DLTs were reported at 9.6 mg/m² BIW: increased GGT (grade 4) and fatigue (grade 3), both of which resolved following cessation or interruption of treatment with BT1718.

The most common related adverse event class reported to date has been grade 1-3 gastrointestinal disorders (18/28 patients), including nausea, diarrhea and vomiting.

Grade 1-2 related peripheral neuropathy occurred more commonly with increasing dose.

With once weekly dosing, BT1718 appears tolerable at dose levels tested, with manageable toxicity.

No objective responses (RECIST 1.1) observed to date in this unselected population. 6/20 patients had stable disease at the 8 week timepoint; one patient had ˜14% decrease in target lesions at cycle 6, with ˜45% decrease in one lesion. Mean number of cycles received was 3 months (n=28).

Summary of adverse events: Table C shows drug-related events reported by ≥15% patients.

TABLE C
Drug-related events reported by ≥ 15% patients.

| | | Number of patients, |
|---|---|---|
| SYSTEM ORGAN CLASS | Preferred term | n = 28 (frequency) |
| BLOOD AND LYMPHATIC SYSTEM | Anaemia | 7 (25.0%) |
| DISORDERS | | |
| GASTROINTESTINAL DISORDERS | Diarrhea | 10 (35.7%) |
| | Nausea | 12 (42.9%) |
| | Vomiting | 9 (32.1%) |
| GENERAL DISORDERS AND | Fatigue | 9 (32.1%) |
| ADMINISTRATION SITE | | |
| CONDITIONS | | |
| INVESTIGATIONS | Alanine aminotransferase increased | 8 (28.6%) |
| | Aspartate aminotransferase increased | 9 (32.1%) |
| | Blood alkaline phosphatase increased | 5 (17.9%) |
| | Gamma-glutamyltransferase increased | 5 (17.9%) |
| METABOLISM AND NUTRITION | Decreased appetite | 9 (32.1%) |
| DISORDERS | | |
| NERVOUS SYSTEM DISORDERS | Lethargy | 6 (21.4%) |
| | Neuropathy peripheral | 9 (32.1%) |

### Pharmacokinetic Results

BT1718 AUC was approximately dose proportional over the range 0.6-25 mg/m² and cycle 2 values were consistent with cycle 1.

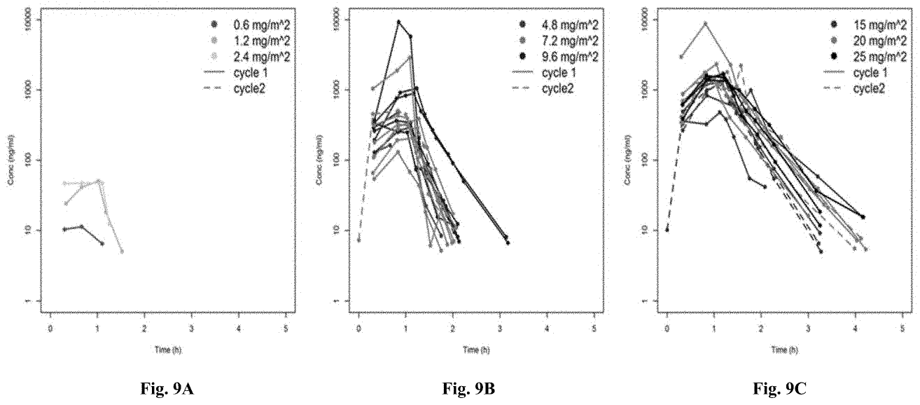

Spaghetti plots: BT1718 plasma concentration vs time after first doses in cycles 1 & 2 are depicted in FIG. 9A, FIG. 9B, and FIG. 9C.

Mean (±SD) plasma clearance (CLp) was 32.3 (±24.9) L/h, with mean (±SD) volume of distribution (Vss) of 22.9 (37.4) L, resulting in a terminal half-life (t1/2) of 0.2 to 1 h.

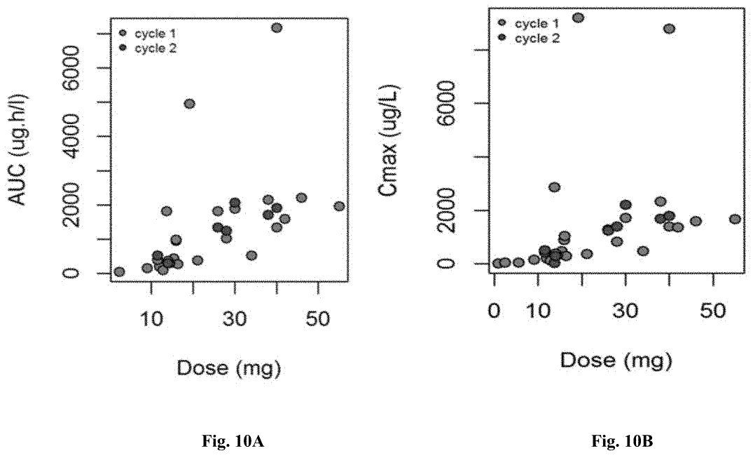

Scatter plots: BT1718 AUC vs dose is depicted in FIG. 10A and BT1718 Cmax vs dose is depicted in FIG. 10B.

Data for all patients: CLp=10 mL/min/kg; t½=18 min.

Data for cycle 1 only: CLp=12 mL/min/kg; t½=18 min.

AUC of BT1718 increased with dose following a 1 h IV infusion, and is consistent between Cycles 1 and 2.

RP2D for twice weekly dosing determined as 7.2 mg/m². A greater total BT1718 dose per cycle was achieved using once weekly dosing (dose escalation ongoing at 32 mg/m²); therefore, RP2D used in the expansion phase will be for once weekly schedule only.

Once weekly RP2D will be assessed for efficacy in patients selected for tumoral MT1-MM/P expression.

### Example 2

### PK Results of BT1718 Dosed in Man

### PK Results for Cohorts 1-5

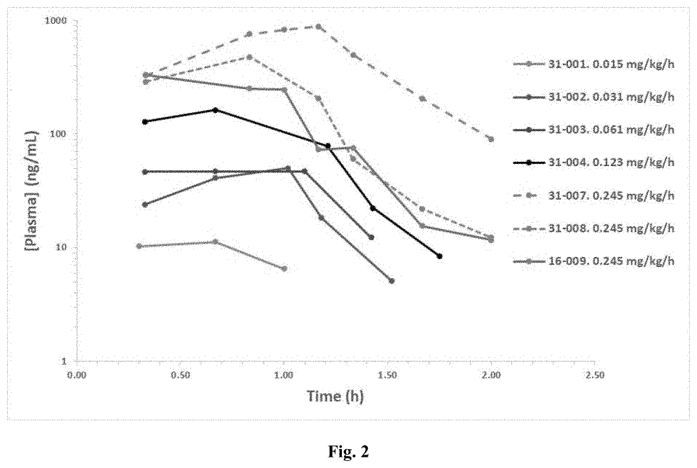

Results of BT1718 dosed in cohorts 1-5 are depicted in FIG. 2.

The preliminary clinical pharmacokinetic data for BT1718 following a 1 hour intravenous infusion to patients are shown in Table 13 below.

TABLE 13
Preliminary clinical pharmacokinetic data for BT1718
following a 1 h intravenous infusion to patients.

| Patient | Dose | CLp | Vss | t½ |
|---|---|---|---|---|
| Code | (mg/m²) | (mL/min/kg) | (L/kg) | (min) |
| 31-001 | 0.6 | n/c | n/c | n/c |
| 31-002 | 1.2 | 12.5 | 0.23 | 6 |
| 31-003 | 2.4 | 18.4 | 0.30 | 6 |
| 31-004 | 4.8 | 13.3 | 0.19 | 10 |
| 31-007 | 9.6 | 4.4 | 0.15 | 16 |
| 31-008 | 9.6 | 10.2 | 0.18 | 17 |
| 16-009 | 9.6 | 13.3 | 0.17 | 14 |

n/c = not calculated as insufficient data
Pharmacokinetic parameters:
CLp = total plasma clearance
Vss-volume of distribution at steady-state
t½ = terminal plasma half-life

BT1718 plasma assay is fully validated with sufficient dynamic range. Systemic exposure is measured at starting dose. It has been found that plasma concentrations increase with dose, and that plasma concentrations in line with preclinical data (rat and primate).

### Example 3

### Increased Tumor Cell Death Following BT1718 Dosing

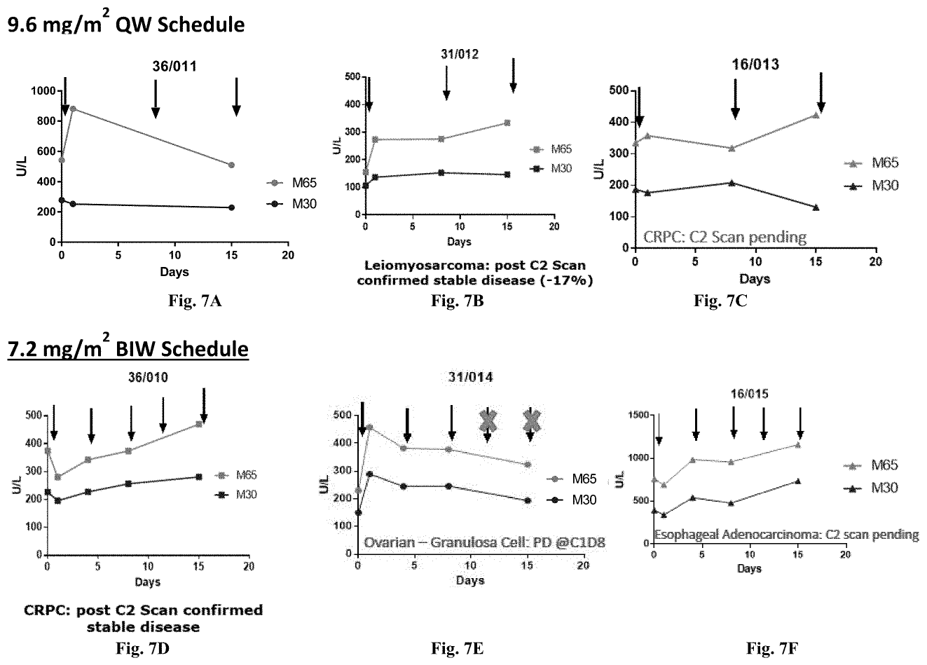

BT1718 increases tumor epithelial cell apoptotic/necrotic death, as shown by M30 and M65 assay (FIG. 7A-FIG. 7F). Measurement was done in serum on C1D1 (pre-dose) & 24 hrs (post dose) then pre—each dose in cycle 1. Changes in cell death markers were observed in all (5/5) patients at highest evaluated dose. All 5 patients had SD at first disease assessment (2 representative curves presented). The data may represent an early pharmacodynamic marker of BT1718 antitumor activity.

### Example 4

### Analysis of DM1 Delivery to Tumor in Patients

A quantitative high performance liquid chromatography-tandem mass spectrometry (LC-MS/MS) method for the quantification of DM1 in tissue samples was developed.

DM1 was administered as complexed with a bicyclic peptide in BT1781. In order to determine the concentration of the free DM1 the conjugated molecule was reduced and subsequently derivatized with vinyl pyridine.

The same sample preparation procedure used for PK determination in the GLP pre-clinical studies, is applied for the determination of DM1 in tissue samples, In short: after tissue homogenization samples were reduced with TCEP prior derivatization by vinyl-pyridine. Derivatization is needed to stabilize the highly reactive thiol group in DM1.

Unless otherwise specified, the denomination DM1 in this example will be used to indicate the vinyl pyridine-derivatized DM1.

A general overview of the main steps of the method can be outlined as follows:
1) Tissue homogenization
2) Reduction/alkylation
3) Protein precipitation
4) Analysis of the sample extract by LC-MS/MS

The homogenization method was optimized as follows:
1) Thaw the tissue samples at room temperature.
2) Weight the desired amount of tissue in a Precellys 2 mL Reinforced tube on an Analytical Balance.
3) Use the weight of the tissue to calculate the volume of solution. Ratio tissue/solution has to be 1:30 W(mg)/V(μL). Homogenization solution: (NaCl (0.9%):SDS (0.2 mg/mL) (50:50 v/v %))
4) Add the calculated amount of solution to the tissue.
5) Vortex for approximately 5 seconds.
6) Pre-cool the samples in a wet ice bath for approximately 8 minutes
7) Run the Precellys-24 Dual homogenizer at 5000 rpm in five sessions of 40 seconds.
8) Centrifuge the homogenate samples 3 minutes at 3000 rpm at 4° C. using a 5810-R centrifuge.

SDS was added at a concentration of 0.1 mg/mL final to increase recovery. The yield in homogenates in the presence of SDS was higher when compared to the yield of homogenate in the presence of ammonium acetate buffer. As a control, Milli Q, SDS and ammonium acetate buffers were used.

Analysis of three patient tumor samples indicated delivery of DM1 to tumor in two patients consistent with non-clinical models.

Preliminary analysis of total DM1 levels in tumor indicate localization of DM1 at tumor.

### REFERENCES

1. Pahwa, S., M. J. Stawikowski, and G. B. Fields, Monitoring and Inhibiting MT1-MMP during Cancer Initiation and Progression. Cancers (Basel), 2014. 6(1): p. 416-35.
2. Kessenbrock, K., V. Plaks, and Z. Werb, Matrix Metalloproteinases: Regulators of the Tumor Microenvironment. Cell, 2010. 141(1): p. 52-67.
3. Wang, Y. Z., et al., MMP-14 overexpression correlates with poor prognosis in non-small cell lung cancer. Tumor Biol, 2014. 35(10): p. 9815-21.
4. Zhou, H., et al., Significance of semaphorin-3A and MMP-14 protein expression in non-small cell lung cancer. Oncology Letters, 2014. 7(5): p. 1395-1400.
5. Laudanski, P., et al., Increased serum level of membrane type 1-matrix metalloproteinase (MT1-MMP/MMP-14) in patients with breast cancer. Folia Histochem Cytobiol, 2010. 48(1): p. 101-3.
6. Têtu, B., et al., The influence of MMP-14, TIMP-2 and MMP-2 expression on breast cancer prognosis. Breast Cancer Research, 2006. 8(3): p. R28-R28.
7. Akanuma, N., et al., MicroRNA-133a regulates the mRNAs of two invadopodia-related proteins, FSCN1 and MMP14, in esophageal cancer. Br J Cancer, 2014. 110(1): p. 189-198.
8. Peng, C. W., et al., Combined features based on MT1-MMP expression, CD11b+ immunocytes density and LNR predict clinical outcomes of gastric cancer. J Transl Med, 2013. 11: p. 153.
9. Landolt, L., et al., Clear Cell Renal Cell Carcinoma is linked to Epithelial-to-Mesenchymal Transition and to Fibrosis. Physiol Rep, 2017. 5(11).
10. Vandenbroucke, R. E. and C. Libert, Is there new hope for therapeutic matrix metalloproteinase inhibition? Nat Rev Drug Discov, 2014. 13(12): p. 904-927.
11. CRUK. http://www.cancerresearchuk.org/about-cancer/type/lung-cancer/about/types-of-lung-cancer. 2014.
12. Scagliotti, G. V., et al., Phase III study comparing cisplatin plus gemcitabine with cisplatin plus pemetrexed in chemotherapy-naive patients with advanced-stage non-small-cell lung cancer. J Clin Oncol, 2008. 26(21): p. 3543-51.
13. Schiller, J. H., et al., Comparison of four chemotherapy regimens for advanced non-small-cell lung cancer. N Engl J Med, 2002. 346(2): p. 92-8.
14. Borghaei, H., et al., Nivolumab versus Docetaxel in Advanced Nonsquamous Non-Small-Cell Lung Cancer. N Engl J Med, 2015. 373(17): p. 1627-39.
15. Brahmer, J., et al., Nivolumab versus Docetaxel in Advanced Squamous-Cell Non-Small-Cell Lung Cancer. N Engl J Med, 2015. 373(2): p. 123-35.
16. Fehrenbacher, L., et al., Atezolizumab versus docetaxel for patients with previously treated non-small-cell lung cancer (POPLAR): a multicentre, open-label, phase 2 randomised controlled trial. Lancet, 2016. 387(10030): p. 1837-46.
17. Hanna, N., et al., Randomized phase III trial of pemetrexed versus docetaxel in patients with non-small-cell lung cancer previously treated with chemotherapy. J Clin Oncol, 2004. 22(9): p. 1589-97.
18. Herbst, R. S., et al., Pembrolizumab versus docetaxel for previously treated, PD-L1-positive, advanced non-small-cell lung cancer (KEYNOTE-010): a randomised controlled trial. Lancet, 2016. 387(10027): p. 1540-50.
19. CRUK, http://www.cancerresearchuk.org/about-cancer/cancers-in-general/cancer-questions/triple-negative-breast-cancertriple. 2014.
20. Gradishar, W. J., et al., Significantly longer progression-free survival with nab-paclitaxel compared with docetaxel as first-line therapy for metastatic breast cancer. J Clin Oncol, 2009. 27(22): p. 3611-9.
21. Jones, S. E., et al., Randomized phase III study of docetaxel compared with paclitaxel in metastatic breast cancer. J Clin Oncol, 2005. 23(24): p. 5542-51.
22. Tutt. A, et al., The TNT trial: A randomized phase III trial of carboplatin (C) compared with docetaxel (D) for patients with metastatic or recurrent locally advanced triple negative or BRCA1/2 breast cancer (CRUK/07/012), in San Antonio Breast Cancer Symposium 2014: San Antonio.
23. Pivot, X., et al., Pooled analyses of eribulin in metastatic breast cancer patients with at least one prior chemotherapy. Ann Oncol, 2016. 27(8): p. 1525-31.
24. Cortes, J., et al., Phase II study of the halichondrin B analog eribulin mesylate in patients with locally advanced or metastatic breast cancer previously treated with an anthracycline, a taxane, and capecitabine. J Clin Oncol, 2010. 28(25): p. 3922-8.
25. Yardley, D. A., et al., EMERGE: A Randomized Phase II Study of the Antibody-Drug Conjugate Glembatumumab Vedotin in Advanced Glycoprotein NMB-Expressing Breast Cancer. J Clin Oncol, 2015. 33(14): p. 1609-19.
26. Bardia, A., et al., Efficacy and Safety of Anti-Trop-2 Antibody Drug Conjugate Sacituzumab Govitecan (I1/MU-132) in Heavily Pretreated Patients With Metastatic Triple-Negative Breast Cancer. Journal of Clinical Oncology, 2017. 0(0): p. JCO.2016.70.8297.
27. Krop, I. E., et al., Trastuzumab emtansine versus treatment of physician's choice for pretreated HER2-positive advanced breast cancer (TH3RESA): a randomised, open-label, phase 3 trial. Lancet Oncol, 2014. 15(7): p. 689-99.
28. Francis, M., et al., Bone and Soft Tissue Sarcomas: UK Incidence and Survival:1996 to 2010, 2013 (v2).
29. CRUK, http://about-cancer.cancerresearchuk.org/about-cancer/soft-tissue-sarcoma. 2015.
30. Van Glabbeke, M., et al., Progression-free rate as the principal end-point for phase II trials in soft-tissue sarcomas. Eur J Cancer, 2002. 38(4): p. 543-9.
31. Poon, K. A., et al., Preclinical safety profile of trastuzumab emtansine (T-DM1): mechanism of action of its cytotoxic component retained with improved tolerability. Toxicol Appl Pharmacol, 2013. 273(2): p. 298-313.
32. FDA, CENTER FOR DRUG EVALUATION AND RESEARCH, Application No. 25427Orig1s000 Pharmacology review Non-clinical pharmacology and toxicology studies to support Kadcyla (adotrastuzumab emtansine) BLA 125427 for the treatment of patients with HER2-positive, metastatic breast cancer, J. K. Leighton, Editor 2013.
33. Shen, B. Q., et al., Non-Clinical Disposition and Metabolism of DM1, a Component of Trastuzumab Emtansine (T-DM1), in Sprague Dawley Rats. Drug Metab Lett, 2015. 9(2): p. 119-31.
34. Seely, J. C. and K. S. Frazier, Regulatory Forum Opinion Piece*: Dispelling Confusing Pathology Terminology: Recognition and Interpretation of Selected Rodent Renal Tubule Lesions. Toxicol Pathol, 2015. 43(4): p. 457-63.
35. Grisold, W., G. Cavaletti, and A. J. Windebank, Peripheral neuropathies from chemotherapeutics and targeted agents: diagnosis, treatment, and prevention. Neuro Oncol, 2012. 14 Suppl 4: p. iv45-54.
36. Tolcher, A. W., et al., Cantuzumab mertansine, a maytansinoid immunoconjugate directed to the CanAg antigen: a phase I, pharmacokinetic, and biologic correlative study. J Clin Oncol, 2003. 21(2): p. 211-22.
37. Rodon, J., et al., Cantuzumab mertansine in a three-times a week schedule: a phase I and pharmacokinetic study. Cancer Chemother Pharmacol, 2008. 62(5): p. 911-9.
38. Shah, M. H., et al., Phase I study of IMGN901, a CD56-targeting antibody-drug conjugate, in patients with CD56-positive solid tumors. Investigational New Drugs, 2016. 34: p. 290-299.
39. EMA/749228, Assessment report: Kadcyla International non-proprietary name: Trastuzumab emtansine, Procedure No. EMEA/H/C/002389/0000. European Medicine Agency. EMA/749228/2013 Committee for Medicinal Products for Human Use (CHMP), 2013.
40. Sibaud, V., et al., [Pigmentary disorders induced by anticancer agents. part I: chemotherapy]. Ann Dermatol Venereol, 2013. 140(3): p. 183-96.
41. CRUK. http://www.cancerresearchuk.org/about-cancer/cancers-in-general/treatment/cancer-drugs/side-effects/your-mouth-and-cancer-drugs. 2013.
42. Cabanillas, F., et al., Phase I study of maytansine using a 3-day schedule. Cancer Treat Rep, 1978. 62(3): p. 425-8.
43. Eagan, R. T., et al., Early clinical study of an intermittent schedule for maytansine (NSC-153858): brief communication. J Natl Cancer Inst, 1978. 60(1): p. 93-6.
44. Chahinian, A. P., et al., Phase I study of weekly maytansine given by iv bolus or 24-hour infusion. Cancer Treat Rep, 1979. 63(11-12): p. 1953-60.
45. Chabner, B. A., et al., Initial clinical trials of maytansine, an antitumor plant alkaloid. Cancer Treat Rep, 1978. 62(3): p. 429-33.
46. Thake, D. C., et al., Toxicity of Maytansine (NSC 153858) in Dogs and Monkeys. PB—U.S. National Technical Information Service, 1975. February 1975, 244628.
47. Mugera, G. M. and J. M. Ward, Acute toxicity of maytansine in F344 rats. Cancer Treat Rep, 1977. 61(7): p. 1333-8.
48. Jackson, D. and D. Stover, Using the Lessons Learned From the Clinic to Improve the Preclinical Development of Antibody Drug Conjugates. Pharmaceutical Research, 2015. 32(11): p. 3458-3469.
49. Shah, M. A., Update on Metastatic Gastric and Esophageal Cancers. Journal of Clinical Oncology, 2015. 33(16): p. 1760-1769.
50. Kang, Y. K., et al., A randomized, open-label, multicenter, adaptive phase 2/3 study of trastuzumab emtansine (T-DM1) versus a taxane (TAX) in patients (pts) with previously treated HER2-positive locally advanced or metastatic gastric/gastroesophageal junction adenocarcinoma (LA/MGC/GEJC). in 2016 Gastrointestinal Cancers Symposium2016, J Clin Oncol. p. suppl 4S; abstr 5.
51. Diamantis, N. and U. Banerji, Antibody-drug conjugates—an emerging class of cancer treatment. British Journal of Cancer, 2016. 114: p. 362-367.
52. Stathis, A., et al., A Phase I Study of IMGN529, an Antibody-Drug Conjugate (ADC) Targeting CD37, in Adult Patients with Relapsed or Refractory B-Cell Non-Hodgkin's Lymphoma (NHL). Blood, 2014. 124(21): p. 1760-1760.
53. Wang, J., W. C. Shen, and J. L. Zaro, Antibody-Drug Conjugates: The 21st Century Magic Bullets for Cancer2015: Springer International Publishing.
54. Galsky, M. D., et al., Phase I trial of the prostate-specific membrane antigen-directed immunoconjugate MLN2704 in patients with progressive metastatic castration-resistant prostate cancer. J Clin Oncol, 2008. 26(13): p. 2147-54.
55. Milowsky, M. I., et al., Phase 1/2 multiple ascending dose trial of the prostate-specific membrane antigen-targeted antibody drug conjugate MLN2704 in metastatic castration-resistant prostate cancer. Urol Oncol, 2016. 34(12): p. 530 e15-530 e21.
56. Kreidieh, F. Y., H. A. Moukadem, and N. S. El Saghir, Overview, prevention and management of chemotherapy extravasation. World Journal of Clinical Oncology, 2016. 7(1): p. 87-97.
57. Carpenito-Moyet, L. J., Nursing Care Plans and Documentation: Nursing Diagnoses and Colloborative Problems2005: Lippincott Williams & Wilkins.
58. Li, Y., et al., The overexpression membrane type 1 matrix metalloproteinase is associated with the progression and prognosis in breast cancer. American Journal of Translational Research, 2015. 7(1): p. 120-127.

### APPENDICES

### Appendix 1: WHO Performance Scale (Table 14)

TABLE 14
WHO Performance Scale

| Activity Performance Description | Score |
|---|---|
| Fully active, able to carry out all normal activity without restriction. | 0 |
| Restricted in physically strenuous activity but ambulatory and able to carry out | 1 |
| work of a light or sedentary nature, for example, light housework, office work. | |
| Ambulatory and capable of all self-care, but unable to carry out any work | 2 |
| activities. Up and about more than 50% of waking hours. | |
| Capable of only limited self-care. Confined to bed or chair more than 50% of | 3 |
| waking hours. | |
| Completely disabled. Cannot carry out any self-care. Totally confined to bed | 4 |
| or chair. | |

### Appendix 2: Assessment of Disease Response

Assessment of disease response in this study should be performed according to the RECIST criteria specified below.

New Response Evaluation Criteria in Solid Tumors (RECIST Criteria):

Revised RECIST guideline (version 1.1)

E. A. Eisenhauer et al. (2009) European Journal of Cancer 45: 228-247

Note that this is an abridged version of the RECIST criteria. Please refer to the above article for detailed appendices and if in doubt.

Measurability of Tumor at Baseline

### Definitions

At baseline, tumor lesions/lymph nodes will be categorised measurable or non-measurable as follows:

Measurable

Tumor lesions: Must be accurately measured in at least one dimension (longest diameter in the plane of measurement is to be recorded) with a minimum size of:

10 mm by CT scan (CT scan slice thickness no greater than 5 mm; see Appendix II on imaging guidance).

10 mm caliper measurement by clinical exam (lesions which cannot be accurately measured with calipers should be recorded as non-measurable).

20 mm by Chest X-Ray

Malignant lymph nodes: To be considered pathologically enlarged and measurable, a lymph node must be 15 mm in the short axis when assessed by CT scan (CT scan slice thickness recommended to be no greater than 5 mm). At baseline and in follow-up, only the short axis will be measured and followed.

Non-Measurable

All other lesions, including small lesions (longest diameter <10 mm or pathological lymph nodes with 10 to <15 mm short axis) as well as truly non-measurable lesions. Lesions considered truly non measurable include: leptomeningeal disease, ascites, pleural or pericardial effusion, inflammatory breast disease, lymphangitic involvement of skin or lung, abdominal masses/abdominal organomegaly identified by physical exam that is not measurable by reproducible imaging techniques.

Special Considerations Regarding Lesion Measurability

Bone lesions, cystic lesions, and lesions previously treated with local therapy require particular comment:

Bone Lesions:

Bone scan, PET scan or plain films are not considered adequate imaging techniques to measure bone lesions. However, these techniques can be used to confirm the presence or disappearance of bone lesions.

Lytic bone lesions or mixed lytic-blastic lesions, with identifiable soft tissue components, that can be evaluated by cross sectional imaging techniques such as CT or MRI can be considered as measurable lesions if the soft tissue component meets the definition of measurability described above.

Blastic bone lesions are non-measurable.

Cystic Lesions:

Lesions that meet the criteria for radiographically defined simple cysts should not be considered as malignant lesions (neither measurable nor non-measurable) since they are, by definition, simple cysts.

‘Cystic lesions’ thought to represent cystic metastases can be considered as measurable lesions, if they meet the definition of measurability described above. However, if non-cystic lesions are present in the same patient, these are preferred for selection as target lesions.

Lesions with Prior Local Treatment:

Tumor lesions situated in a previously irradiated area, or in an area subjected to other loco regional therapy, are usually not considered measurable unless there has been demonstrated progression in the lesion. Study protocols should detail the conditions under which such lesions would be considered measurable.

Specifications by Methods of Measurements

Measurement of Lesions

All measurements should be recorded in metric notation, using callipers if clinically assessed. All baseline evaluations should be performed as close as possible to the treatment start and never more than 4 weeks before the beginning of the treatment.

Method of Assessment

The same method of assessment and the same technique should be used to characterise each identified and reported lesion at baseline and during follow-up. Imaging based evaluation should always be done rather than clinical examination unless the lesion(s) being followed cannot be imaged but are assessable by clinical exam.

Clinical Lesions:

Clinical lesions will only be considered measurable when they are superficial and ≥10 mm diameter as assessed using callipers (e.g. skin nodules). For the case of skin lesions, documentation by colour photography including a ruler to estimate the size of the lesion is suggested. As noted above, when lesions can be evaluated by both clinical exam and imaging, imaging evaluation should be undertaken since it is more objective and may also be reviewed at the end of the study.

Chest X-Ray:

Chest CT is preferred over chest X-ray, particularly when progression is an important endpoint, since CT is more sensitive than X-ray, particularly in identifying new lesions. However, lesions on chest X ray may be considered measurable if they are clearly defined and surrounded by aerated lung.

CT, MRI:

CT is the best currently available and reproducible method to measure lesions selected for response assessment. This guideline has defined measurability of lesions on CT scan based on the assumption that CT slice thickness is 5 mm or less. When CT scans have slice thickness greater than 5 mm, the minimum size for a measurable lesion should be twice the slice thickness. MRI is also acceptable in certain situations (e.g. for body scans). More details concerning the use of both CT and MRI for assessment of objective tumor response evaluation are provided in the publication from Eisenhauer et al.

Ultrasound:

Ultrasound is not useful in assessment of lesion size and should not be used as a method of measurement. Ultrasound examinations cannot be reproduced in their entirety for independent review at a later date and, because they are operator dependent, it cannot be guaranteed that the same technique and measurements will be taken from one assessment to the next (described in greater detail in Appendix II). If new lesions are identified by ultrasound in the course of the study, confirmation by CT or MRI is advised. If there is concern about radiation exposure at CT, MRI may be used instead of CT in selected instances.

Endoscopy, Laparoscopy:

The utilization of these techniques for objective tumor evaluation is not advised. However, they can be useful to confirm complete pathological response when biopsies are obtained or to determine relapse in trials where recurrence following complete response or surgical resection is an endpoint.

Tumor Markers:

Tumor markers alone cannot be used to assess objective tumor response. If markers are initially above the upper normal limit, however, they must normalize for a patient to be considered in complete response.

Cytology, Histology:

These techniques can be used to differentiate between PR and CR in rare cases if required by protocol (for example, residual lesions in tumor types such as germ cell tumors, where known residual benign tumors can remain). When effusions are known to be a potential adverse effect of treatment (e.g. with certain taxane compounds or angiogenesis inhibitors), the cytological confirmation of the neoplastic origin of any effusion that appears or worsens during treatment can be considered if the measurable tumor has met criteria for response or stable disease in order to differentiate between response (or stable disease) and PD.

Tumor Response Evaluation

Assessment of Overall Tumor Burden and Measurable Disease

To assess objective response or future progression, it is necessary to estimate the overall tumor burden at baseline and use this as a comparator for subsequent measurements. Measurable disease is defined by the presence of at least one measurable lesion (as detailed above).

Baseline Documentation of ‘Target’ and ‘Non-Target’ Lesions

When more than one measurable lesion is present at baseline all lesions up to a maximum of five lesions total (and a maximum of two lesions per organ) representative of all involved organs should be identified as target lesions and will be recorded and measured at baseline (this means in instances where patients have only one or two organ sites involved a maximum of two and four lesions respectively will be recorded). For evidence to support the selection of only five target lesions, see analyses on a large prospective database in the article by Bogaerts et al. Target lesions should be selected on the basis of their size (lesions with the longest diameter), be representative of all involved organs, but in addition should be those that lend themselves to reproducible repeated measurements. It may be the case that, on occasion, the largest lesion does not lend itself to reproducible measurement in which circumstance the next largest lesion which can be measured reproducibly should be selected. An example in FIG. 3 of the publication by Eisenhauer et al.

Lymph nodes merit special mention since they are normal anatomical structures which may be visible by imaging even if not involved by tumor. Pathological nodes which are defined as measurable and may be identified as target lesions must meet the criterion of a short axis of ≥15 mm by CT scan. Only the short axis of these nodes will contribute to the baseline sum. The short axis of the node is the diameter normally used by radiologists to judge if a node is involved by solid tumor. Nodal size is normally reported as two dimensions in the plane in which the image is obtained (for CT scan this is almost always the axial plane; for MRI the plane of acquisition may be axial, sagittal or coronal). The smaller of these measures is the short axis. For example, an abdominal node which is reported as being 20 mm×30 mm has a short axis of 20 mm and qualifies as a malignant, measurable node. In this example, 20 mm should be recorded as the node measurement. All other pathological nodes (those with short axis ≥10 mm but <15 mm) should be considered non-target lesions. Nodes that have a short axis <10 mm are considered non-pathological and should not be recorded or followed.

A sum of the diameters (longest for non-nodal lesions, short axis for nodal lesions) for all target lesions will be calculated and reported as the baseline sum diameters. If lymph nodes are to be included in the sum, then as noted above, only the short axis is added into the sum. The baseline sum diameters will be used as reference to further characterise any objective tumor regression in the measurable dimension of the disease.

All other lesions (or sites of disease) including pathological lymph nodes should be identified as non-target lesions and should also be recorded at baseline. Measurements are not required and these lesions should be followed as ‘present’, ‘absent’, or in rare cases ‘unequivocal progression’ (more details to follow). In addition, it is possible to record multiple non-target lesions involving the same organ as a single item on the case record form (e.g. ‘multiple enlarged pelvic lymph nodes’ or ‘multiple liver metastases’).

Response Criteria

Evaluation of Target Lesions

Complete Response (CR): Disappearance of all target lesions. Any pathological lymph nodes (whether target or non-target) must have reduction in short axis to <10 mm.

Partial Response (PR): At least a 30% decrease in the sum of diameters of target lesions, taking as reference the baseline sum diameters.

Progressive Disease (PD): At least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm. (Note: the appearance of one or more new lesions is also considered progression).

Stable Disease (SD): Neither sufficient shrinkage to qualify for PR nor sufficient increase to qualify for PD, taking as reference the smallest sum diameters while on study.

Special Notes on the Assessment of Target Lesions

Lymph Nodes.

Lymph nodes identified as target lesions should always have the actual short axis measurement recorded (measured in the same anatomical plane as the baseline examination), even if the nodes regress to below 10 mm on study. This means that when lymph nodes are included as target lesions, the ‘sum’ of lesions may not be zero even if complete response criteria are met, since a normal lymph node is defined as having a short axis of <10 mm. Case report forms or other data collection methods may therefore be designed to have target nodal lesions recorded in a separate section where, in order to qualify for CR, each node must achieve a short axis <10 mm. For PR, SD and PD, the actual short axis measurement of the nodes is to be included in the sum of target lesions.

Target Lesions that Become ‘too Small to Measure’.

While on study, all lesions (nodal and non-nodal) recorded at baseline should have their actual measurements recorded at each subsequent evaluation, even when very small (e.g. 2 mm). However, sometimes lesions or lymph nodes which are recorded as target lesions at baseline become so faint on CT scan that the radiologist may not feel comfortable assigning an exact measure and may report them as being ‘too small to measure’. When this occurs it is important that a value be recorded on the case report form. If it is the opinion of the radiologist that the lesion has likely disappeared, the measurement should be recorded as 0 mm. If the lesion is believed to be present and is faintly seen but too small to measure, a default value of 5 mm should be assigned. (Note: It is less likely that this rule will be used for lymph nodes since they usually have a definable size when normal and are frequently surrounded by fat such as in the retroperitoneum; however, if a lymph node is believed to be present and is faintly seen but too small to measure, a default value of 5 mm should be assigned in this circumstance as well). This default value is derived from the 5 mm CT slice thickness (but should not be changed with varying CT slice thickness). The measurement of these lesions is potentially non-reproducible, therefore providing this default value will prevent false responses or progressions based upon measurement error. To reiterate, however, if the radiologist is able to provide an actual measure, that should be recorded, even if it is below 5 mm.

Lesions that Split or Coalesce on Treatment:

When non-nodal lesions ‘fragment’, the longest diameters of the fragmented portions should be added together to calculate the target lesion sum. Similarly, as lesions coalesce, a plane between them may be maintained that would aid in obtaining maximal diameter measurements of each individual lesion. If the lesions have truly coalesced such that they are no longer separable, the vector of the longest diameter in this instance should be the maximal longest diameter for the ‘coalesced lesion’.

Evaluation of Non-Target Lesions

While some non-target lesions may actually be measurable, they need not be measured and instead should be assessed only qualitatively at the time points specified in the protocol.

Complete Response (CR): Disappearance of all non-target lesions and normalisation of tumor marker level. All lymph nodes must be non-pathological in size (<10 mm short axis).

Non-CR/Non-PD: Persistence of one or more non-target lesion(s) and/or maintenance of tumor marker level above the normal limits.

Progressive Disease (PD): Unequivocal progression (see comments below) of existing non-target lesions. (Note: the appearance of one or more new lesions is also considered progression).

Special Notes on Assessment of Progression of Non-Target Disease

The concept of progression of non-target disease requires additional explanation as follows:

When the patient also has measurable disease.

In this setting, to achieve ‘unequivocal progression’ on the basis of the non-target disease, there must be an overall level of substantial worsening in non-target disease such that, even in presence of SD or PR in target disease, the overall tumor burden has increased sufficiently to merit discontinuation of therapy. A modest ‘increase’ in the size of one or more non-target lesions is usually not sufficient to quality for unequivocal progression status. The designation of overall progression solely on the basis of change in non-target disease in the face of SD or PR of target disease will therefore be extremely rare.

When the Patient has Only Non-Measurable Disease.

This circumstance arises in some phase III trials when it is not a criterion of study entry to have measurable disease. The same general concepts apply here as noted above, however, in this instance there is no measurable disease assessment to factor into the interpretation of an increase in non-measurable disease burden. Because worsening in non-target disease cannot be easily quantified (by definition: if all lesions are truly non-measurable) a useful test that can be applied when assessing patients for unequivocal progression is to consider if the increase in overall disease burden based on the change in non-measurable disease is comparable in magnitude to the increase that would be required to declare PD for measurable disease: i.e. an increase in tumor burden representing an additional 73% increase in ‘volume’ (which is equivalent to a 20% increase diameter in a measurable lesion). Examples include an increase in a pleural effusion from ‘trace’ to ‘large’, an increase in lymphangitic disease from localised to widespread, or may be described in protocols as ‘sufficient to require a change in therapy’. If ‘unequivocal progression’ is seen, the patient should be considered to have had overall PD at that point. While it would be ideal to have objective criteria to apply to non-measurable disease, the very nature of that disease makes it impossible to do so; therefore the increase must be substantial.

New Lesions

The appearance of new malignant lesions denotes disease progression; therefore, some comments on detection of new lesions are important. There are no specific criteria for the identification of new radiographic lesions; however, the finding of a new lesion should be unequivocal: i.e. not attributable to differences in scanning technique, change in imaging modality or findings thought to represent something other than tumor (for example, some ‘new’ bone lesions may be simply healing or flare of pre-existing lesions). This is particularly important when the patient's baseline lesions show partial or complete response. For example, necrosis of a liver lesion may be reported on a CT scan report as a ‘new’ cystic lesion, which it is not.

A lesion identified on a follow-up study in an anatomical location that was not scanned at baseline is considered a new lesion and will indicate disease progression. An example of this is the patient who has visceral disease at baseline and while on study has a CT or MRI brain ordered which reveals metastases. The patient's brain metastases are considered to be evidence of PD even if he/she did not have brain imaging at baseline.

If a new lesion is equivocal, for example because of its small size, continued therapy and follow-up evaluation will clarify if it represents truly new disease. If repeat scans confirm there is definitely a new lesion, then progression should be declared using the date of the initial scan.

While fluorodeoxyglucose (FDG)-positron emission tomography (PET) response assessments need additional study, it is sometimes reasonable to incorporate the use of FDG-PET scanning to complement CT scanning in assessment of progression (particularly possible ‘new’ disease). New lesions on the basis of FDG-PET imaging can be identified according to the following algorithm:

a. Negative FDG-PET at baseline, with a positive FDG-PET at follow-up is a sign of PD based on a new lesion.

b. No FDG-PET at baseline and a positive FDG-PET at follow-up:

If the positive FDG-PET at follow-up corresponds to a new site of disease confirmed by CT, this is PD.

If the positive FDG-PET at follow-up is not confirmed as a new site of disease on CT, additional follow-up CT scans are needed to determine if there is truly progression occurring at that site (if so, the date of PD will be the date of the initial abnormal FDG-PET scan). A ‘positive’ FDG-PET scan lesion means one which is FDG avid with an uptake greater than twice that of the surrounding tissue on the attenuation corrected image.

If the positive FDG-PET at follow-up corresponds to a pre-existing site of disease on CT that is not progressing on the basis of the anatomic images, this is not PD.

Evaluation of Best Overall Response

The best overall response is the best response recorded from the start of the study treatment until the end of treatment taking into account any requirement for confirmation. Should a response not be documented until after the end of therapy in this trial, post-treatment assessments may be considered in the determination of best overall response as long as no alternative anti-cancer therapy has been given. The patient's best overall response assignment will depend on the findings of both target and non-target disease and will also take into consideration the appearance of new lesions.

Time Point Response

It is assumed that at each protocol-specified time point, a response assessment occurs. Table 15 provides a summary of the overall response status calculation at each time point for patients who have measurable disease at baseline.

When patients have non-measurable (therefore non-target) disease only, Table 16 is to be used.

Missing Assessments and Inevaluable Designation

When no imaging/measurement is done at all at a particular time point, the patient is not evaluable (NE) at that time point. If only a subset of lesion measurements are made at an assessment, usually the case is also considered NE at that time point, unless a convincing argument can be made that the contribution of the individual missing lesion(s) would not change the assigned time point response. This would be most likely to happen in the case of PD. For example, if a patient had a baseline sum of 50 mm with three measured lesions and at follow-up only two lesions were assessed, but those gave a sum of 80 mm, the patient will have achieved PD status, regardless of the contribution of the missing lesion.

Best Overall Response: All Time Points

The best overall response is determined once all the data for the patient is known.

Best response in this trial is defined as the best response across all time points (for example, a patient who has SD at first assessment, PR at second assessment, and confirmed after 4 weeks, and PD on last assessment has a best overall response of confirmed PR). All CRs or PRs must be confirmed after at least 4 weeks, until which time they are “unconfirmed” CRs or PRs. The date of PR or CR is then the initial date when response was first noted, rather than the date of the confirmatory scan. When SD is believed to be best response, it must also meet the protocol specified minimum time from baseline. If the minimum time is not met when SD is otherwise the best time point response, the patient's best response depends on the subsequent assessments. For example, a patient who has SD at first assessment, PD at second and does not meet minimum duration for SD, will have a best response of PD. The same patient lost to follow-up after the first SD assessment would be considered inevaluable. A ‘positive’ FDG-PET scan lesion means one which is FDG avid with an uptake greater than twice that of the surrounding tissue on the attenuation corrected image.

TABLE 15
Time point response: patients with target (+/−non-target) disease.

| | | New | Overall |
|---|---|---|---|
| Target lesions | Non-target lesions | lesions | response |
| CR | CR | No | CR |
| CR | Non-CR/non-PD | No | PR |
| CR | Not evaluated | No | PR |
| PR | Non-PD or not all | No | PR |
| | evaluated | | |
| SD | Non-PD or not all | No | SD |
| | evaluated | | |
| Not all evaluated | Non-PD | No | NE |
| PD | Any | Yes or No | PD |
| Any | PD | Yes or No | PD |
| Any | Any | Yes | PD |

CR = complete response,
PR = partial response,
SD = stable disease,
PD = progressive disease, and
NE = inevaluable.

TABLE 16
Time point response: patients with non-target disease only.

| Non-target lesions | New lesions | Overall response |
|---|---|---|
| CR | No | CR |
| Non-CR/non-PD | No | Non-CR/non-PD(a) NE |
| Not all evaluated | No | PD |
| Unequivocal PD | Yes or No | PD |
| Any | Yes | PD |

CR = complete response,
PD = progressive disease, and
NE = inevaluable.
(a) ‘Non-CR/non-PD’ is preferred over ‘stable disease’ for non-target disease since SD is increasingly used as endpoint for assessment of efficacy in some trials so to assign this category when no lesions can be measured is not advised.

TABLE 17
Best overall response when confirmation of CR and PR required.

| | Overall | |
|---|---|---|
| Overall | response | |
| response | Subsequent | |
| First time | time | |
| point | point(s) | BEST overall response |
| CR | CR | CR |
| CR | PR | SD, PD or PR(a) |
| CR | SD | SD provided minimum criteria for SD |
| | | duration met, otherwise, PD |
| CR | PD | SD provided minimum criteria for SD |
| | | duration met, otherwise PD |
| CR | NE | SD provided minimum criteria for SD |
| | | duration met, otherwise NE |
| PR | CR | PR |
| PR | PR | PR |
| PR | SD | SD |
| PR | PD | SD provided minimum criteria for SD |
| | | duration met, otherwise, PD |
| PR | NE | SD provided minimum criteria for SD |
| | | duration met, otherwise NE |
| NE | NE | NE |

CR = complete response,
PR = partial response,
SD = stable disease,
PD = progressive disease, and
NE = inevaluable.
(a) If a CR is truly met at first time point, then any disease seen at a subsequent time point, even disease meeting PR criteria relative to baseline, makes the disease PD at that point (since disease must have reappeared after CR). Best response would depend on whether minimum duration for SD was met. However, sometimes ‘CR’ may be claimed when subsequent scans suggest small lesions were likely still present and in fact the patient had PR, not CR at the first time point. Under these circumstances, the original CR should be changed to PR and the best response is PR.

Special Notes on Response Assessment

When nodal disease is included in the sum of target lesions and the nodes decrease to ‘normal’ size (<10 mm), they may still have a measurement reported on scans. This measurement should be recorded even though the nodes are normal in order not to overstate progression should it be based on increase in size of the nodes. As noted earlier, this means that patients with CR may not have a total sum of ‘zero’ on the case report form (CRF).

Patients with a global deterioration of health status requiring discontinuation of treatment without objective evidence of disease progression at that time should be reported as ‘symptomatic deterioration’. Every effort should be made to document objective progression even after discontinuation of treatment. Symptomatic deterioration is not a descriptor of an objective response: it is a reason for stopping study therapy. The objective response status of such patients is to be determined by evaluation of target and non-target disease as shown in Tables 1 to 3.

Conditions that define ‘early progression, early death and in evaluability’ are study specific and should be clearly described in each protocol (depending on treatment duration, treatment periodicity).

In some circumstances it may be difficult to distinguish residual disease from normal tissue. When the evaluation of complete response depends upon this determination, it is recommended that the residual lesion be investigated (fine needle aspirate/biopsy) before assigning a status of complete response. FDG-PET may be used to upgrade a response to a CR in a manner similar to a biopsy in cases where a residual radiographic abnormality is thought to represent fibrosis or scarring.

For equivocal findings of progression (e.g. very small and uncertain new lesions; cystic changes or necrosis in existing lesions), treatment may continue until the next scheduled assessment. If at the next scheduled assessment, progression is confirmed, the date of progression should be the earlier date when progression was suspected.

Duration of Overall Response

The duration of overall response is measured from the time measurement criteria are first met for CR/PR (whichever is first recorded) until the first date that recurrent or progressive disease is objectively documented (taking as reference for progressive disease the smallest measurements recorded on study).

The duration of overall complete response is measured from the time measurement criteria are first met for CR until the first date that recurrent disease is objectively documented.

Duration of Stable Disease

Stable disease is measured from the start of the treatment (in randomised trials, from date of randomisation) until the criteria for progression are met, taking as reference the smallest sum on study (if the baseline sum is the smallest, this is the reference for calculation of PD).

### Appendix 3: New York Heart Association (NYHA) Scale

TABLE 18
New York Heart Association (NYHA) Scale

| Class | Description |
|---|---|
| Class I- | patients with cardiac disease but without resulting limitation of physical |
| | activity; ordinary physical activity does not cause undue dyspnoea (or |
| | fatigue, palpitation or anginal pain) |
| Class II- | patients with cardiac disease resulting in slight limitation of physical |
| | activity; they are comfortable at rest; ordinary physical activity results in |
| | dyspnoea (or fatigue, palpitation or anginal pain) |
| Class III- | patients with cardiac disease resulting in marked limitations of physical |
| | activity; they are comfortable at rest; less than ordinary physical activity |
| | causes dyspnoea (or fatigue, palpitation or anginal pain) |
| Class IV- | patients with cardiac disease resulting in inability to carry out physical |
| | activity without discomfort; symptoms of dyspnoea (or of angina) may be |
| | present even at rest; if any physical activity is undertaken, discomfort is |
| | increased. |

### Appendix 4: Table of Abbreviations and Definitions of Terms

TABLE 19
Abbreviations and Definitions of Terms

| | Abbreviation | Definition |
|---|---|---|
| A | ABPI | Association of the British Pharmaceutical Industry |
| | ADA | Antidrug antibodies |
| | ADC | Antibody Drug Conjugates |
| | AE | adverse event |
| | ALP | alkaline phosphatase |
| | ALT | alanine aminotransferase |
| | ALK | Anaplastic lymphoma Kinase |
| | ANC | absolute neutrophil count |
| | AST | aspartate aminotransferase |
| | AUC | area under the curve |
| B | BDC | Bicycle Drug Conjugate |
| | BP | blood pressure |
| | BSA | body surface area |
| C | CDD | Centre for Drug Development |
| | CI | Chief Investigator |
| | cfDNA | Cell free DNA |
| | CLT | total body clearance |
| | Cₘₐₓ | maximum observed plasma concentration |
| | CNS | central nervous system |
| | CR | complete response |
| | CRA | Clinical Research Associate |
| | CRUK | Cancer Research UK |
| | CSM | Clinical Study Manager |
| | CT | computerised tomography |
| | CTCAE | Common Terminology Criteria for Adverse Events |
| D | Day | calendar day |
| | DLT | dose limiting toxicity |
| | DM1 | cytotoxic agent (N2’-deacetyl-N2’-(3-mercapto-l-oxopropyl)- |
| | | maytansine) |
| E | ECG | electrocardiogram |
| | eCRF | electronic case report form |
| | EDC | electronic data capture |
| | ELISA | Enzyme linked immunosorbent assay |
| | EORTC | European Organization for Research and Treatment of Cancer |
| F | FDG | Fluorodeoxyglucose |
| | FFPE | Formalin-fixed paraffin embedded |
| | FIH | First in human |
| G | GCP | Good Clinical Practice |
| | GFR | glomerular filtration rate |
| | GI | Gastrointestinal |
| | GIST | Gastrointestinal stromal tumor |
| | GLP | Good Laboratory Practice |
| | GMP | Good Manufacturing Practice |
| H | h | hour |
| | Hb | hemoglobin |
| | HCG | human chorionic gonadotropin |
| | HED | human equivalent dose |
| | HER2 | human epidermal receptor 2 |
| | HIV | human immunodeficiency virus |
| | HNSTD | highest non-severely toxic dose |
| | HRA | Health Research Authority |
| I | IB | Investigators Brochure |
| | ICD | Informed consent document |
| | ICH GCP | International Conference on Harmonization of Good Clinical |
| | | Practice |
| | IHC | Immunohistochemistry |
| | IMP | investigational medicinal product |
| | ITF | Investigator Trial File |
| L | LC/MSMC | Liquid chromatography-mass spectrometry mass spectrometry |
| M | MAD | maximum administered dose |
| | MT1-MMP | membrane type 1 matrix metalloproteinase |
| | min | minute(s) |
| | MHRA | Medicines and Healthcare products Regulatory Agency |
| | MRI | magnetic resonance imaging |
| | MTD | maximum tolerated dose |
| N | NCI | National Cancer Institute |
| | NSCLC | Non-Small Cell Lung Cancer |
| | NYHA | New York Heart Association |
| O | ORR | objective response rate |
| | OS | overall survival |
| P | PD | progressive disease |
| | PD-1 | programmed cell death protein 1 |
| | PDX | patient-derived xenograft |
| | PET | positron emission tomography |
| | PI | Principal Investigator |
| | PFS | progression free survival |
| | PK | pharmacokinetic |
| | PR | partial response |
| R | REC | Research Ethics Committee |
| | RECIST | Response Evaluation Criteria in Solid Tumors |
| | RP2D | recommended Phase II dose |
| S | SAE | serious adverse event |
| | SD | stable disease |
| | SDV | source data verification |
| | SOC | standard of care |
| | SOP | standard operating procedure |
| | SUSAR | suspected unexpected serious adverse (drug) reaction |
| T | T½ | half-life |
| | T-DM1 | ado-trastuzumab emtansine |
| | TEAE | treatment-emergent adverse events |
| | TK | Toxicokinetics |
| | TMA | Tumor microarray |
| | TNBC | Triple Negative Breast Cancer |
| U | UK | United Kingdom |
| | ULN | upper limit of normal |
| | USM | urgent safety measure |
| V | Vd_{SS} | steady state volume of distribution |
| W | WFI | water for injection |
| | WBC | white blood cell |
| | WHO | World Health Organization |

1-40. (canceled)

41. A method for treating an advanced solid tumor in a human patient, comprising administering to said patient BT1718, or a pharmaceutically acceptable salt, twice weekly at a dose of about 4.8-9.6 mg/m² by intravenous infusion.

42. The method of claim 41, wherein the dose is about 4.8 mg/m².

43. The method of claim 41, wherein the dose is about 7.2 mg/m².

44. The method of claim 41, wherein the dose is about 9.6 mg/m².

45. The method of claim 41, wherein BT1718, or a pharmaceutically acceptable salt, is administered at an infusion rate of about 0.015-0.245 mg/kg/h.

46. The method of claim 41, wherein BT1718, or a pharmaceutically acceptable salt, is administered at day 1 and 4 of each week.

47. The method of claim 41, wherein each treatment cycle is four weeks.

48. The method of claim 47, wherein BT1718, or a pharmaceutically acceptable salt, is administered in weeks 1, 2, and 3 in each treatment cycle.

49. The method of claim 48, wherein BT1718, or a pharmaceutically acceptable salt, is administered at days 1, 4, 8, 11, 15, and 18 of each treatment cycle.

50. The method of claim 41, wherein the advanced solid tumor is non-small cell lung cancer.

51. The method of claim 41, wherein the advanced solid tumor is triple negative breast cancer.

52. The method of claim 41, wherein the advanced solid tumor is sarcoma.

53. A method for treating an advanced solid tumor in a human patient, comprising administering to said patient BT1718, or a pharmaceutically acceptable salt, once a week at a dose of about 15-20 mg/m² by intravenous infusion.

54. The method of claim 53, wherein the dose is about 15 mg/m².

55. The method of claim 53, wherein the dose is about 18 mg/m².

56. The method of claim 53, wherein the dose is about 20 mg/m².

57. The method of claim 53, wherein BT1718, or a pharmaceutically acceptable salt, is administered at an infusion rate of about 0.015-0.245 mg/kg/h.

58. The method of claim 53, wherein the advanced solid tumor is non-small cell lung cancer.

59. The method of claim 53, wherein the advanced solid tumor is triple negative breast cancer.

60. The method of claim 53, wherein the advanced solid tumor is sarcoma.
